(19) United States
(12) Patent Application Publication
(10) Pub. No.: US 2025/0352626 A1
(43) Pub. Date: Nov. 20, 2025

(54) IMMUNE TOLERANCE INDUCTION TO VIRAL CAPSIDS

(71) Applicant: University of Florida Research Foundation, Incorporated, Gainesville, FL (US)

(72) Inventors: Barry John BYRNE, Gainesville, FL (US); Manuela CORTI, Alachua, FL (US)

(21) Appl. No.: 18/870,780

(22) PCT Filed: Jun. 9, 2023
(86) PCT No.: PCT/US2023/068178

§ 371 Date: Dec. 2, 2024

Related U.S. Application Data
(60) Provisional application No. 63/351,059, filed on Jun. 10, 2022.

(51) Int. Cl. A61K 39/00; A61K 39/23; A61K 48/00; A61P 37/06; C12N 7/00; C12N 15/86

(52) U.S. Cl. CPC A61K 39/001; A61K 39/23; A61K 48/0058; A61P 37/06; C12N 7/00; C12N 15/86; A61K 2039/55555; C12N 2740/15034; C12N 2740/15043; C12N 2750/14122; C12N 2750/14134; C12N 2750/14143

(57) ABSTRACT

Described are viral vectors, compositions, kits, and methods or using the vectors, compositions, kits to modulate immune response in a subject. The viral vectors include therapeutic recombinant adeno-associated viruses (rAAVs) and tolerance inducing gene therapy vectors. The therapeutic rAAVs and tolerance inducing gene therapy vectors can be used to deliver one or more therapeutic nucleic acids to the subject. The tolerance inducing gene therapy vectors induce immune-specific tolerance to the therapeutic rAAVs to improve efficacy of the therapeutic rAAVs and allow for multiple administrations of the therapeutic rAAVs with little or no associated immune response to the therapeutic rAAVs. The therapeutic rAAVs can be used to administer a therapeutic effect to the subject.

### CROSS-REFERENCE TO RELATED APPLICATION

This application claims the benefit of U.S. Provisional Application No. 63/351,059, filed Jun. 10, 2022, which is incorporated herein by reference.

### SEQUENCE LISTING

This application incorporates by reference the Sequence Listing contained in the following XML file being submitted concurrently herewith: File Name: T18819_SeqList.xml, was created May 31, 2023, and is 105 KB in size.

### BACKGROUND

Gene therapy provides successful therapy for a number of diseases or conditions. However, immune responses to viral vectors can present potential limitations on repeated administration of a therapeutic viral vector.

### SUMMARY

Described are methods for inducing immune tolerance to a viral vector such as a therapeutic recombinant adeno-associated virus (rAAV). The therapeutic rAAV can encode a therapeutic nucleic acid. Viral gene therapy may require repeat administrations of the therapeutic rAAV. However, some patients may develop an immune reaction to subsequent administrations of the therapeutic rAAV, which can limit the use of the therapeutic rAAV to deliver a therapeutic nucleic acid. As described herein, administering to the subject a tolerance-inducing gene therapy vector comprising a nucleic acid encoding at least a portion of a capsid protein of the therapeutic rAAV induces specific immune tolerance to the therapeutic rAAV, thereby providing for effective repeat administration of the therapeutic rAAV. In some embodiments, the tolerance-inducing gene therapy vector can be any vector capable of targeting the liver and expressing the at least a portion of the capsid protein of the therapeutic rAAV in the liver. In some embodiments, the tolerance-inducing gene therapy vector can be any vector capable of targeting hematopoietic stem cells (HSC) and expressing the at least a portion of the capsid protein of the therapeutic rAAV in the HSC. The tolerance-inducing gene therapy vector can be a viral vector or a non-viral vector. A viral vector can be, but is not limited to, a lentiviral vector. A non-viral vector can be, but is not limited to, a lipoplex, a polyplex, a lipopolyplex, a polymersome, or a nanoparticle. In some embodiments, the gene therapy vector comprises a lipid nanoparticle (LNP). Administering to the subject the tolerance-inducing gene therapy vector comprising a nucleic acid sequence encoding at least a portion of a capsid of the therapeutic rAAV induces specific immune tolerance to the therapeutic rAAV, thereby providing for effective repeat administration of the therapeutic rAAV. The nucleic acid can be, but is not limited to, a viral vector, a DNA (such as a plasmid), or an RNA (such as an mRNA).

Described are methods of inducing immune tolerance to a therapeutic rAAV in a subject comprising: administering to a subject an effective amount of a tolerance-inducing gene therapy vector wherein the tolerance-inducing gene therapy vector comprises a nucleic acid sequence encoding at least a portion of a capsid protein of the therapeutic rAAV operably linked to a promoter; wherein the tolerance-inducing gene therapy vector is capable of delivering the nucleic acid to a liver cell or a HSC, wherein the nucleic acid encoding the at least a portion of the capsid protein of the therapeutic rAAV is expressed in the liver or HSC, thereby inducing immune tolerance to the therapeutic rAAV. The promoter can be, but is not limited to a constitutive promoter, an inducible promoter, a liver-specific or hepatocyte-specific promoter, a HSC-specific promoter, or a synthetic promoter. A liver-specific promoter can be, but is not limited to, an albumin promoter, an alpha-1-antitrypsin promoter, transthyretin (TTR) promoter, and apolipoprotein E (apoE) promoter, a hepatitis B virus core protein promoter, or a promoter comprising the nucleic acid sequence of SEQ ID NO: 3 or a nucleic acid sequence having at least 90% sequence identity to SEQ ID NO: 3. In some embodiments, the use of a liver-specific promoter results in expression of the at least a portion of a capsid protein solely or substantially in the liver. In some embodiments, the use of a HSC-specific promoter results in expression of the at least a portion of a capsid protein solely or substantially in HSC. In some embodiments, the promoter is an EF1 promoter. In some embodiments, the at least a portion of the capsid protein of the therapeutic rAAV comprises an immunogenic fragment of the capsid protein. The capsid protein can be, but is not limited to, a VP1 capsid protein, a VP2 capsid protein, and/or a VP3 capsid protein. Inducing immune tolerance to the therapeutic rAAV can comprise one or more of: inducing regulatory T cells (Tregs) specific to the therapeutic rAAV, reducing cytotoxic CD8+ T cell response to the therapeutic rAAV, reducing the level of pre-existing antibodies to the therapeutic rAAV, and reducing production of antibodies against the therapeutic rAAV. In some embodiments, the tolerance-inducing gene therapy vector is administered systemically. The tolerance-inducing gene therapy vector can be administered to a subject that has previously been administered the therapeutic rAAV or to a subject that has not been previously administered the therapeutic rAAV. The tolerance-inducing gene therapy vector can be administered to a subject simultaneously with administering the therapeutic rAAV.

Described are methods of inducing immune tolerance to a therapeutic rAAV in a subject comprising: administering to a subject an effective amount of a tolerance-inducing lentiviral vector, comprising a nucleic acid sequence encoding at least a portion of a capsid protein of the therapeutic rAAV, wherein the nucleic acid sequence is operably linked to a promoter; and wherein the lentiviral vector is capable of infecting a liver cell or a hematopoietic stem cell (HSC), and expressing the at least a portion of the capsid protein of the therapeutic rAAV in the liver or HSC, thereby inducing immune tolerance to the therapeutic rAAV. In some embodiments, the tolerance-inducing lentiviral vector is administered systemically. The promoter can be, but is not limited to a constitutive promoter, an inducible promoter, a liver-specific or hepatocyte-specific promoter, a HSC-specific promoter, or a synthetic promoter. A liver-specific promoter can be, but is not limited to, an albumin promoter, an alpha-1-antitrypsin promoter, transthyretin (TTR) promoter, and apolipoprotein E (apoE) promoter, a hepatitis B virus core protein promoter, or a promoter comprising the nucleic acid sequence of SEQ ID NO: 3 or a nucleic acid sequence having at least 90% sequence identity to SEQ ID NO: 3. In some embodiments, the use of a liver-specific promoter results in expression of the at least a portion of a capsid protein solely or substantially in the liver. In some embodiments, the use of a HSC-specific promoter results in expression of the at least a portion of a capsid protein solely or substantially in the HSC. In some embodiments, the promoter is an EF1 promoter. In some embodiments, the at least a portion of the capsid protein of the therapeutic rAAV comprises an immunogenic fragment of the capsid protein. The capsid protein can be, but is not limited to, a VP1 capsid protein, a VP2 capsid protein, and/or a VP3 capsid protein. Inducing immune tolerance to the therapeutic rAAV can comprise one or more of: inducing regulatory T cells (Tregs) specific to the therapeutic rAAV, reducing cytotoxic CD8+ T cell response to the therapeutic rAAV, reducing the level of pre-existing antibodies to the therapeutic rAAV, and reducing production of antibodies against the therapeutic rAAV. In some embodiments, the tolerance-inducing lentiviral vector is administered systemically. The tolerance-inducing lentiviral vector can be administered to a subject that has previously been administered the therapeutic rAAV or to a subject that has not been previously administered the therapeutic rAAV. The tolerance-inducing lentiviral vector can be administered to a subject simultaneously with administering the therapeutic rAAV.

Described are methods of inducing immune tolerance to a therapeutic rAAV in a subject comprising: infecting one or more HSC obtained from the subject with a tolerance-inducing lentiviral vector comprising a nucleic acid sequence encoding at least a portion of a capsid protein of the therapeutic rAAV, wherein the nucleic acid sequence is operably linked to a promoter, and expressing the at least a portion of the capsid protein of the therapeutic rAAV in the HSC, and administering the HSC expressing the capsid protein of the therapeutic rAAV to the subject, thereby inducing immune tolerance to the therapeutic rAAV. The promoter can be, but is not limited to a constitutive promoter, an inducible promoter, a HSC-specific promoter, or a synthetic promoter. In some embodiments, the at least a portion of the capsid protein of the therapeutic rAAV comprises an immunogenic fragment of the capsid protein. The capsid protein can be, but is not limited to, a VP1 capsid protein, a VP2 capsid protein, and/or a VP3 capsid protein. Inducing immune tolerance to the therapeutic rAAV can comprise one or more of: inducing regulatory T cells (Tregs) specific to the therapeutic rAAV, reducing cytotoxic CD8+ T cell response to the therapeutic rAAV, reducing the level of pre-existing antibodies to the therapeutic rAAV, and reducing production of antibodies against the therapeutic rAAV. The HSC expressing the capsid protein of the therapeutic rAAV can be administered to a subject that has previously been administered the therapeutic rAAV or to a subject that has not been previously administered the therapeutic rAAV. The HSC expressing the capsid protein of the therapeutic rAAV can be administered to a subject simultaneously with administering the therapeutic rAAV.

The methods disclosed herein relate to methods of tolerizing a subject to AAV viral antigens thereby allowing for repeat administration of AAV as a therapy. In some embodiments, the methods comprise administering to the subject an effective amount of a therapeutic rAAV and administering to the subject a tolerance-inducing gene therapy vector comprising a nucleotide sequence encoding at least a portion of a capsid protein of the therapeutic AAV, wherein the nucleotide sequence is operably linked to a promoter suitable for expression in the liver or HSC, wherein the at least a portion of the capsid protein of the therapeutic rAAV is immunogenic, and wherein expression of the at least a portion of the capsid protein in the liver or HSC induces specific immune tolerance to the therapeutic rAAV in the subject. The tolerance-inducing gene therapy vector can be, but is not limited to, a non-viral vector. The non-viral vector can be, but is not limited to a LNP. The promoter can be, but is not limited to a constitutive promoter, an inducible promoter, a liver-specific or hepatocyte-specific promoter, a HSC-specific promoter, or a synthetic promoter. A liver-specific promoter can be, but is not limited to, an albumin promoter, an alpha-1-antitrypsin promoter, a hepatitis B virus core protein promoter, or a promoter comprising the nucleic acid sequence of SEQ ID NO: 3 or a nucleic acid sequence having at least 90%, at least 95%, at least 96%, at least 97%, or at least 98% sequence identity to SEQ ID NO: 3. In some embodiments, the promoter is an EF1 promoter. Optionally, combinations of promoters can be used to drive enhanced expression of the transgene. The nucleic acid can be, but is not limited to, a DNA (such as a plasmid), or an RNA (such as an mRNA). In some embodiments, administering the tolerance-inducing gene therapy vector reduces or eliminates a humoral immune response against the therapeutic rAAV or against the capsid protein of the therapeutic rAAV. In some embodiments, administration of the tolerance-inducing gene therapy vector results in one or more of: increased or induced rAAV-specific regulatory T cells (Tregs), reduced cytotoxic CD8+ T cell response to the therapeutic rAAV, reduced level of pre-existing antibodies to the therapeutic rAAV, and reduction in the production of antibodies against the therapeutic rAAV. In some embodiments, the therapeutic AAV comprises a therapeutic nucleic acid sequence. The therapeutic nucleic acid sequence can be, but is not limited to, an expressible gene. The expressible gene can encode an RNA or a therapeutic polypeptide.

The methods disclosed herein relate to methods of tolerizing a subject to AAV viral antigens thereby allowing for repeat administration of AAV as a therapy. In some embodiments, the methods comprise administering to the subject an effective amount of therapeutic rAAV and administering a tolerance-inducing lentiviral vector comprising a nucleotide sequence encoding at least a portion of a capsid protein of the therapeutic AAV, wherein the nucleotide sequence is operably linked to a promoter suitable for expression in the liver or HSC, wherein the at least a portion of the capsid protein of the therapeutic rAAV is immunogenic, and wherein expression of the at least a portion of the capsid protein in the liver or HSC induces specific immune tolerance to the therapeutic rAAV in the subject. In some embodiments, HSC are infected by the lentivirus ex vivo and the infected HSC expressing the capsid protein of the therapeutic rAAV are administered to the subject. The promoter can be, but is not limited to constitutive promoter, an inducible promoter, a liver-specific or hepatocyte-specific promoter, a HSC-specific promoter, or a synthetic promoter. A liver-specific promoter can be, but is not limited to, an albumin promoter, an alpha-1-antitrypsin promoter, a hepatitis B virus core protein promoter, or a promoter comprising the nucleic acid sequence of SEQ ID NO: 3 or a nucleic acid sequence having at least 90%, at least 95%, at least 96%, at least 97%, or at least 98% sequence identity to SEQ ID NO: 3. In some embodiments, the promoter is an EF1 promoter. Optionally, combinations of promoters can be used to drive enhanced expression of the transgene. In some embodiments, administering the tolerance-inducing lentiviral vector reduces or eliminates a humoral immune response against a therapeutic rAAV or against the capsid protein or the therapeutic rAAV. In some embodiments, administration of the tolerance-inducing lentiviral vector results in one or more of: increased or induced rAAV-specific regulatory T cells (Tregs), reduced cytotoxic CD8+ T cell response to the therapeutic rAAV, reduced level of pre-existing antibodies to the therapeutic rAAV, and reduction in the production of antibodies against the therapeutic rAAV. In some embodiments, the therapeutic AAV comprises a therapeutic nucleic acid sequence. The therapeutic nucleic acid sequence can be, but is not limited to, an expressible gene. The expressible gene can encode an RNA or a therapeutic polypeptide.

Described are methods of inducing in a subject an immune response against an antigen. The antigen can be a tumor antigen or an antigen from a pathogen. The methods comprise: (a) administering to the subject an effective dose of a therapeutic rAAV comprising a first nucleotide sequence encoding an antigenic peptide, wherein the first nucleotide sequence is operably linked to a first promoter; and (b) administering to the subject an effective dose of a tolerance-inducing gene therapy vector, wherein the tolerance-inducing gene therapy vector comprises a second nucleic acid sequence encoding at least a portion of a capsid protein of the therapeutic rAAV, wherein the second nucleic acid sequence is operably linked to a second promoter; and wherein the tolerance-inducing gene therapy vector is capable of delivering the second nucleic acid to a liver cell or HSC, wherein the second nucleic acid is expressed in the liver cell or HSC thereby inducing immune tolerance to the therapeutic rAAV in the subject. The gene therapy vector can be a viral vector or a non-viral vector. The viral vector can be a lentiviral vector. A tolerance-inducing lentiviral vector comprises a liver-targeting lentiviral vector or an HSC-targeting lentiviral vector. In some embodiments, the tolerance-inducing gene therapy vector comprises a non-viral vector. The non-viral vector can be, but is not limited to, a LNP. The pathogen can be, but is not limited to, a virus, a bacteria, a fungus, or a parasite. The first and second promoters can be, but are not limited to, constitutive promoters, inducible promoters, tissue-specific promoters, and synthetic promoters. In some embodiments, the first promoter is a tissue-specific promoter. In some embodiments, the first promoter is a cell-type specific promoter. In some embodiments, the first promoter is a neuronal cell-specific or muscle-specific promoter. In some embodiments, the second promoter is a liver-specific or hepatocyte-specific promoter, or a HSC-specific promoter In some embodiments, the at least a portion of the capsid protein of the therapeutic rAAV is immunogenic. The capsid protein can be, but is not limited to, a VP1 capsid protein, a VP2 capsid protein, and/or a VP3 capsid protein. The immune response can be, but is not limited to, a cellular immune response to the antigen, a humoral immune response to the antigen, enhancing proliferation of antigen-specific cytotoxic T lymphocytes, eliciting generation of anti-antigen antibodies, reducing the likelihood of infection by pathogen containing the antigen, vaccinating the subject a patient against the pathogen, treating cancer, and combinations thereof. The tolerance-inducing gene therapy vector can be administered to the subject prior to, concurrently with, or subsequent to administration of the therapeutic rAAV. In some embodiments, the tolerance-inducing gene therapy vector is administered by intravenous injection. In some embodiments, one or some subsequent effective doses of the therapeutic rAAV are administered to the subject after administration of the first effective dose.

Described are methods of providing a therapeutic nucleic acid to a subject or expressing a therapeutic nucleic acid in a subject. The methods comprise: (a) administering to the subject an effective dose of a therapeutic rAAV comprising a first nucleotide sequence encoding a therapeutic nucleic acid operably linked to a first promoter; and (b) administering to the subject an effective dose of a tolerance-inducing gene therapy vector comprising a second nucleic acid sequence encoding at least a portion of a capsid protein of the therapeutic rAAV operably linked to a second promoter, and wherein the tolerance-inducing gene therapy vector is capable of delivering the second nucleic acid to a liver cell or HSC, wherein the second nucleic acid is expressed in the liver cell or HSC, thereby inducing immune tolerance to the therapeutic rAAV in the subject. The tolerance-inducing gene therapy vector can be a viral vector or a non-viral vector. The viral vector can be, but is not limited to, a lentiviral vector. The tolerance-inducing lentiviral vector comprises a liver-targeted lentiviral vector or a HSC-targeted lentiviral vector. In some embodiments, tolerance-inducing gene therapy vector is a non-viral vector. The non-viral vector can be, but is not limited to, a LNP. The therapeutic nucleic acid sequence can be, but is not limited to, an expressible gene. The expressible gene can encode an RNA or a therapeutic polypeptide. The first and second promoters can be, but are not limited to, constitutive promoters, inducible promoters, tissue-specific promoters, and synthetic promoters. In some embodiments, the first promoter is a neuronal cell-specific or muscle-specific promoter. In some embodiments, the second promoter is a liver-specific or hepatocyte-specific promoter, or a HSC-specific promoter. In some embodiments, the at least a portion of the capsid protein of the therapeutic rAAV is immunogenic. The capsid protein can be, but is not limited to, a VP1 capsid protein, a VP2 capsid protein, and/or a VP3 capsid protein. The tolerance-inducing gene therapy vector can be administered to the subject prior to, concurrently with, or subsequent to administration of the therapeutic rAAV. In some embodiments, the tolerance-inducing gene therapy vector is administered by intravenous injection. In some embodiments, one or some subsequent effective doses of the therapeutic rAAV are administered to the subject after administration of the first effective dose.

Described are methods for modulating an immune response in a host comprising administering to the host an effective amount of a therapeutic recombinant adeno-associated virus (rAAV) comprising a nucleotide sequence encoding all or a portion of a therapeutic protein; and administering to the host a tolerance-inducing gene therapy vector comprising a nucleotide sequence encoding at least a portion of a capsid protein of the therapeutic rAAV operatively linked to a liver-specific promoter, wherein the at least a portion of the capsid protein of the therapeutic rAAV is immunogenic, and wherein expression of the nucleotide sequence encoding the at least a portion of the capsid protein of the therapeutic rAAV in the liver induces specific immune tolerance to the therapeutic rAAV in a subject.

Described are methods for modulating an immune response in a host comprising administering to the host an effective amount of a therapeutic recombinant adeno-associated virus (rAAV) comprising a nucleotide sequence encoding all or a portion of a therapeutic protein; and administering to the host a tolerance-inducing gene therapy vector comprising a nucleotide sequence encoding at least a portion of a capsid protein of the therapeutic rAAV operatively linked to a HSC-specific promoter, wherein the at least a portion of the capsid protein of the therapeutic rAAV is immunogenic, and wherein expression of the nucleotide sequence encoding the at least a portion of the capsid protein of the therapeutic rAAV in the HSC induces specific immune tolerance to the therapeutic rAAV in a subject.

Described are tolerance-inducing gene therapy vectors for inducing immune tolerance to a therapeutic rAAV. A tolerance-inducing gene therapy vector comprises a nucleic acid sequence encoding an immunogenic portion of a capsid protein of the therapeutic rAAV. The nucleic acid sequence is operably linked to a promoter capable of providing expression of the nucleic acid sequence in a liver cell, such as a hepatocyte, or a HSC. The tolerance-inducing gene therapy vector is capable of delivering a nucleic acid to a liver cell or HSC, wherein the nucleic acid is expressed. Expressing the immunogenic portion of the capsid protein in the liver or HSC induces immune tolerance to the therapeutic rAAV. The promoter can be, but is not limited to, a constitutive promoter, an inducible promoter, a liver-specific or hepatocyte-specific promoter, a HSC-specific promoter, or a synthetic promoter. A liver-specific promoter can be, but is not limited to, an albumin promoter, an alpha-1-antitrypsin promoter, a hepatitis B virus core protein promoter, or a promoter comprising the nucleic acid sequence of SEQ ID NO: 3 or a nucleic acid sequence having at least 90%, at least 95%, at least 96%, at least 97%, or at least 98% sequence identity to SEQ ID NO: 3. In some embodiments, the promoter is an EF1 promoter. In some embodiments, the at least a portion of the capsid protein of the therapeutic rAAV comprises at least 10% at least 20%, at least 30%, at least 40%, at least 50%, at least 60%, at least 70%, at least 80%, at least 90%, or 100% of the capsid protein. The capsid protein can comprise one or more of: a VP1 capsid protein, a VP2 capsid protein, and/or a VP3 capsid protein. In some embodiments, the capsid protein comprises the VP3 capsid protein. In some embodiments, the immunogenic portion of the capsid protein has at least 90%, at least 95%, at least 96%, at least 97%, or at least 98% identity to the amino acid sequence of SEQ ID NO: 1. In some embodiments, the immunogenic portion of the capsid protein is encoded by a nucleic acid sequence having at least 90%, at least 95%, at least 96%, at least 97%, or at least 98% identity to SEQ ID NO: 2. The tolerance-inducing gene therapy vector can be a viral vector or a non-viral vector. The viral vector can be a lentiviral vector. In some embodiments, the tolerance-inducing lentiviral vector comprises a liver-targeted lentiviral vector. In some embodiments, the tolerance-inducing lentiviral vector comprises a HSC-targeted lentiviral vector In some embodiments, the gene therapy vector is an LNP. The therapeutic rAAV can be, but is not limited to, serotype 1, serotype 2, serotype 3, serotype 4, serotype 5, serotype 6, serotype 7, serotype 8, serotype 9, serotype 10, serotype 11, rh10, or rh74.

Described are lentiviral vectors for inducing immune tolerance to a therapeutic rAAV. The tolerance-inducing lentiviral vector comprises a nucleic acid sequence encoding an immunogenic portion of a capsid protein of the therapeutic rAAV. The nucleic acid sequence is operably linked to a promoter capable of providing expression of the nucleic acid sequence in a lever cell, such as a hepatocyte, or a HSC. The tolerance-inducing lentiviral vector is capable of infecting a liver cell or HSC and expressing the immunogenic portion of the capsid protein in the liver or HSC thereby inducing immune tolerance to the therapeutic rAAV. The promoter can be, but is not limited to constitutive promoter, an inducible promoter, a liver-specific or hepatocyte-specific promoter, a HSC-specific promoter, or a synthetic promoter. A liver-specific promoter can be, but is not limited to, an albumin promoter, an alpha-1-antitrypsin promoter, a hepatitis B virus core protein promoter, or a promoter comprising the nucleic acid sequence of SEQ ID NO: 3 or a nucleic acid sequence having at least 90%, at least 95%, at least 96%, at least 97%, or at least 98% sequence identity to SEQ ID NO: 3. In some embodiments, the promoter is an EF1 promoter. In some embodiments, the at least a portion of the capsid protein of the therapeutic rAAV comprises at least 10% at least 20%, at least 30%, at least 40%, at least 50%, at least 60%, at least 70%, at least 80%, at least 90%, or 100% of the capsid protein. The capsid protein can comprise one or more of: a VP1 capsid protein, a VP2 capsid protein, and/or a VP3 capsid protein. In some embodiment, the capsid protein comprises the VP3 capsid protein. In some embodiments, the immunogenic portion of the capsid protein has at least 90%, at least 95%, at least 96%, at least 97%, or at least 98% identity to the amino acid sequence of SEQ ID NO: 1. In some embodiments, the immunogenic portion of the capsid protein is encoded by a nucleic acid sequence having at least 90%, at least 95%, at least 96%, at least 97%, or at least 98% identity to SEQ ID NO: 2. The tolerance-inducing rAAV can be, but is not limited to, serotype 1, serotype 2, serotype 3, serotype 4, serotype 5, serotype 6, serotype 7, serotype 8, serotype 9, serotype 10, serotype 11, rh10, or rh74.

Also described are nucleic acids encoding at least a portion of a capsid protein of the therapeutic rAAV and/or therapeutic rAAVs. The nucleic acid can be, but is not limited to, a viral vector, a DNA (such as a plasmid), or an RNA (such as an mRNA). In some embodiments, isolated nucleic acid encoding one or more of the lentiviral vectors, and/or therapeutic rAAVs are provided, including nucleic acids that can be used in the manufacture of the tolerance-inducing lentiviral vectors and/or therapeutic rAAVs. Host cells containing any of the described nucleic acids are also provided. The host cells can be used in the manufacture of tolerance-inducing gene therapy vectors, tolerance-inducing lentiviral vectors and/or therapeutic rAAVs.

Also described are compositions comprising the tolerance-inducing gene therapy vectors and/or therapeutic rAAVs. In some embodiments, the compositions comprise one or more tolerance-inducing gene therapy vectors and one or more carriers or excipients. In some embodiments, the compositions further contain a therapeutic rAAV. In some embodiments, the compositions comprise a tolerance-inducing lentiviral vector and a therapeutic rAAV.

These and other features, aspects, and advantages of the present invention will become better understood with reference to the following description and appended claims.

### BRIEF DESCRIPTION OF THE DRAWINGS

FIG. 1 Plasmid map of an exemplary viral construct used to tolerize a subject against a viral capsid protein, for example the VP3 capsid protein of AAV serotype 1, used as part of a therapeutic AAV vector to allow for repeat administration.

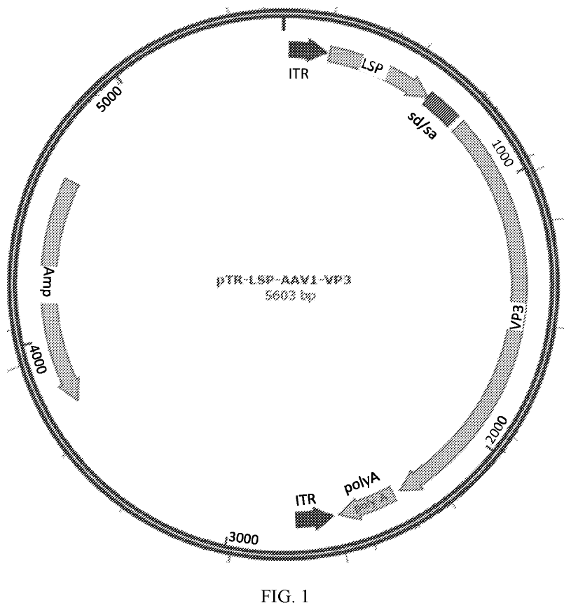

FIG. 2. Schematic of AAV-CMV-SARS2 with AAV2 ITRs and pseudotyped to AAV1.

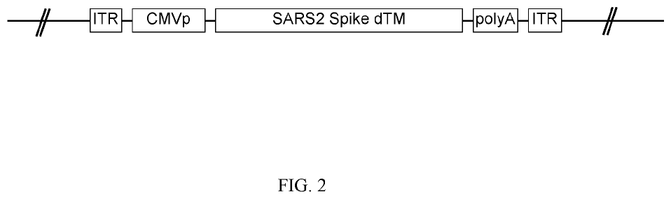

FIG. 3. Plasmid map of an exemplary construct used to tolerize a subject against a viral capsid protein, for example the cap9 capsid protein of AAV (Sequence is provided in SEQ ID NO: 10).

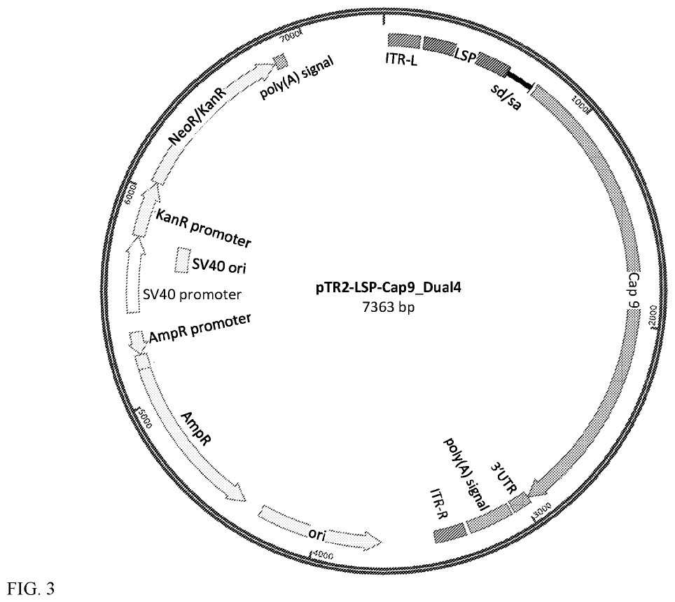

FIG. 4. Plasmid map of an exemplary vector for expressing an AAV capsid protein in the liver. The plasmid, or an mRNA encoded by the plasmid can be delivered to the liver via a non-viral particle, such as an LNP.

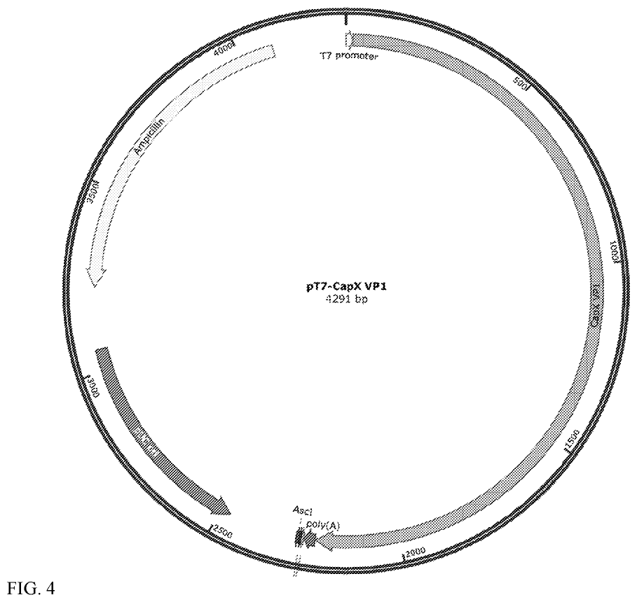

FIG. 5. Plasmid map of an exemplary lentiviral vector expressing an AAV capsid protein suitable for ex-vivo gene therapy to liver cells or hematopoietic stem cells.

FIG. 6. Illustration of pTR2-LSP-VP1 (AAV9) construct.

FIG. 7. Illustration of pTR2-LSP-coVP3 (AAV9) construct.

FIG. 8. Illustration of pTR2-LSP-coVP1 (AAV rh74) construct.

FIG. 9. Illustration of pTR2-LSP-coVP1 (AAV rh74.47.4E) construct.

FIG. 10. Illustration of pTR2-CH19_HA-L-mEFla-coVP1-HA-R (AAV9) construct.

FIG. 11. Illustration of pTR2-H11-LA-mEFla-coVP1-H11-RA. (AAV9) construct.

FIG. 12. Graphs illustrating VP1 expression in HEK293 cells.

FIG. 13. Graph illustrating AAV antibody response in mice treated with tolerance inducing vector.

FIG. 14. Graph illustrating expression of AAV-delivered therapeutic gene (micro-dystrophin) in mice treated with tolerance inducing vector (first bar in each pair is transcription in heart cells, second bar in each pair in skeletal muscle cells.

### DETAILED DESCRIPTION

Reference is made to particular features and/or non-limiting embodiments of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

### I. Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

A “subject” refers to an animal that is the object of treatment, observation, or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, non-human primates, such as monkeys, chimpanzees, and apes, and humans. In some embodiments, the subject is human.

The terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy.

A “vaccine” is a substance(s) or composition(s) used to stimulate an immune response, such as the production of antibodies, and provide immunity against a disease without inducing the disease. Vaccines are often prepared from a causative agent of a disease or a product of the causative agent, such as a polypeptide or nucleic acid encoding the polypeptide. When administered to a subject, a vaccine induces or stimulates an immune response. A vaccine can render a subject resistant or immune to a particular disease or infection. A vaccine can also reduce severity or duration of infection. A vaccine can induce an immune response against a pathogen or a cancer.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter).

The term “effective amount,” as used herein, refers to an amount that is capable of treating or ameliorating a disease or condition or otherwise capable of producing an intended therapeutic effect.

Sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition, or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

Any titles or subheadings used herein are for organization purposes and should not be used to limit the scope of embodiments disclosed herein.

All ranges are to be interpreted as encompassing the endpoints in the absence of express exclusions, such as “not including the endpoints”; thus, for example, “within 10-15” includes the values 10 and 15. The ranges disclosed herein encompass any and all overlap, sub-ranges, and combinations thereof. Language such as “up to,” “at least,” “greater than,” “less than,” “between,” and the like includes the number recited. One skilled in the art will understand that the recited ranges include the end values, as whole numbers in between the end values, and where practical, rational numbers within the range (e.g., the range 5-10 includes 5, 6, 7, 8, 9, and 10, and where practical, values such as 6.8, 9.35, etc.). When the specification discloses a specific value for a parameter, the specification should be understood as alternatively disclosing the parameter at “about” that value. When values are expressed as approximations, by use of the antecedent “about,” it will be understood that the particular value forms a further aspect. For example, if the value “about 10” is disclosed, then “10” is also disclosed. The term “about” or “approximately” indicates within an acceptable error range for the particular value as determined by one of ordinary skill in the art, which will depend in part on how the value is measured or determined, e.g., the limitations of the measurement system. In some embodiments, the term “about” indicates insubstantial variation in a quantity of a component of a composition not having any significant effect on the activity or stability of the composition. In some embodiments, “about” can mean within 1 standard deviation, per the practice in the art. Alternatively, “about” can mean a range of up to 0 to 20%, 0 to 10%, 0 to 5%, or up to 1% of a given value. Where particular values are described in the application and claims, unless otherwise stated the term “about” meaning within an acceptable error range for the particular value should be assumed.

### II. Modulating Immune Response to Gene Therapy Vectors

Described are vectors (viral and non-viral), compositions, kits, and methods or using the vectors, compositions, kits to modulate immune response in a subject. The viral vectors include therapeutic rAAVs and tolerance-inducing gene therapy vectors. The therapeutic rAAVs and tolerance-inducing gene therapy vectors can be used to deliver one or more therapeutic nucleic acids to the subject. The tolerance-inducing gene therapy vectors induce immune-specific tolerance to the therapeutic rAAVs to improve efficacy of the therapeutic rAAVs and/or allow for multiple administrations of the therapeutic rAAVs with reduced or little or no associated immune response to the therapeutic rAAVs. The therapeutic rAAVs can be used to administer a therapeutic effect to the subject.

a. Wild Type Adeno-Associated Virus

Human adeno-associated virus (AAV) is a non-pathogenic parvovirus that only productively replicates in cells co-infected by a helper virus, usually adenovirus or herpes virus. The virus has a wide host range and can productively infect many cell types from a variety of animal species. Sero-epidemiologic studies have shown that most people (50-96%) in the U.S.A. have been exposed to the most common serotype (AAV2), probably as a passenger during a productive adenovirus (Ad) infection. Nevertheless, AAV has not been implicated in any human or animal disease.

AAV binds to cells via a heparan sulfate proteoglycan receptor. Once attached, AAV entry is dependent upon the presence of a co-receptor, either the fibroblast growth factor receptor or avβ5 integrin molecule. In infected cells, the incoming AAV single-stranded DNA (ssDNA) is converted to double-stranded transcriptional template. Cells infected with AAV and a helper virus will undergo productive replication of AAV prior to cell lysis, which is induced by the helper virus rather than AAV. Helper virus encodes proteins or RNA transcripts which are transcriptional regulators and are involved in DNA replication or modify the cellular environment in order to permit efficient viral production. Human cells infected with AAV alone, however, become persistently infected. This latency pathway of wild-type AAV often results in site-specific integration on chromosome 19, the AAVSI site. The AAV genome consists of two 145-nucleotide inverted terminal repeat (ITR) sequences, each an identical palindrome at either terminus of the virus, flanking the two AAV open reading frames (ORFs), rep and cap. AAV rep and cap genes encode the four Rep proteins (Rep 78, 68, 52 and 40) involved in viral DNA replication, resolution of replicative intermediates and generation of single-strand genomes and the three structural proteins (VP1, VP2, and VP3) that make up the viral capsid. The two larger rep proteins (Rep 78 and Rep 68) are required for resolution of the AAV termini during productive infections. They are also capable of binding to the human chromosome 19 target sequence for AAV integration and initiating site-specific integration. Thus, rep-deleted recombinant AAV vectors do not integrate site-specifically, but rather persist as a combination of episomal forms and random-site integrants.

### B. Recombinant Adeno-Associated Viral Vectors

Recombinant AAV (rAAV) vectors are typically produced by replacing the viral coding sequences with transgenes of interest. These vectors have been shown to be highly efficient for gene transfer and expression at a number of different sites in vitro and in vivo. They have consistently mediated stable expression and have been shown to be safe in studies performed in the respiratory tract, the central nervous system, skeletal muscle, liver, and eye. The efficiency of rAAV-mediated transduction has increased as the titer and purity of rAAV preparations has improved. Skeletal muscle is often chosen as the target tissue because it is accessible, efficiently transduced by rAAV vectors, well vascularized, and is able to express and process secreted proteins.

The ITRs from the AAV genome are the only viral sequences required in cis to generate rAAV vectors. Recombinant constructs containing two ITRs bracketing a gene expression cassette of ˜5 kb are converted into a ssDNA vector genome and packaged into AAV particles in the presence of AAV rep and cap gene products and helper functions, usually from an Adenovirus. Methods or production and purification of rAAV are known in the art and are suitable for use with the described rAAVs, compositions, and methods (Zolotukhin et al., Gene Ther. 1999 June; 6 (6): 973-85; Thorne et al., Hum Gene Ther. 2009 July; 20 (7): 707-14; Ayuso et al., Curr Gene Ther. 2010 December; 10 (6): 423-36; Cecchini et al., Hum Gene Ther. 2011 August; 22 (8): 1021-30; Shin et al., Methods Mol Biol. 2012; 798:267-84; Chahal et al., J Virol Methods. 2014 February; 196:163-73; Grieger et al., Mol Ther. 2016 February; 24 (2): 287-297; and Clément and Grieger, Mol Ther Methods Clin Dev. 2016 Mar. 16; 3:16002, each of which is incorporated herein by reference).

Many serotypes of AAV have been cloned and sequenced. Serotypes 1 and 6 share >99% amino acid homology in their capsid proteins. Of the first six AAV serotypes, serotype 2 is best characterized and therefore predominantly used in gene transfer studies. However, according to embodiments disclosed herein, other AAV serotypes can also be used, including AAV9, AAV20, rh74, AAV10, and the like. Comparison of the serotype capsid amino acid sequences suggests that types 1, 2 and 3 share homology across the three capsids in accord with heparan sulfate binding. Direct intramuscular injections of non-rAAV2 vectors, especially rAAV1, transduce skeletal muscle more efficiently and secrete canine factor IX at levels two-to-three logs greater than rAAV2. Because the identical transgene cassette was used in the vector constructs, these results suggest that rAAV1 virions are more efficient for gene delivery to muscle. Furthermore, a humoral response was not detected against the transgene protein secreted by intramuscular injection of the rAAV1 construct, contrasting with the significant humoral response elicited by the transgene protein secreted by myocytes transduced by the rAAV2 construct. The lack of cross-reactivity among neutralizing antibodies of different rAAV serotypes suggests that vector repeat administration using different serotypes of rAAV may be feasible for dose titration.

In general, there are two different approaches for packaging rAAV vectors: “true type” and “pseudotyped” vectors. The former refers to vectors having ITRs, Rep proteins and capsid proteins derived from the same wild-type virus, e.g., AAV2. The latter refers to vectors having ITRs and Rep proteins derived from one serotype virus, and capsid proteins from another, e.g., 2 and 1 (AAV2/1).

In recent years, there have been significant improvements in production and purification of rAAV vectors. The major improvements in production have included enhanced output of the number of particles per cell and the emergence of a number of scalable systems. Several groups have independently found that the use of plasmids to express adenovirus (Ad) helper genes in transient transfection results in greater efficiency of rAAV production than infection with Ad virus, perhaps because of enhanced viability of producer cells or the lack of competition with the helper virus for DNA replication machinery. Another interesting finding is that down-regulation of Rep78/68 relative to Rep52/40 and the capsid proteins results in a greater accumulation of single-stranded DNA genomes and packaged vector DNA. The incorporation of these improvements into transient transfection production protocols has enhanced yields from about 1-10 IU per cell to over 100 IU per cell. Stable producer cell lines and packaging cell lines used in combination with recombinant hybrid AAV-adenoviruses have achieved 100-300 IU per cell. Hybrid AAV-herpes vectors have achieved outputs that approach the 5,000-10,000 IU per cell seen with wtAAV. Overall, these newer methods produce greater vector yields and reduce or eliminate detectable replication competent AAV (rcAAV) contamination.

Early reports comparing the transduction efficiencies and specificities of rAAV vector serotypes relied on CsCl gradients for purification, but this approach can generate vector stocks with large particle: infectious (P: I) ratios. Purification using affinity chromatography, based on identified cellular receptors, is becoming more common and the more physiological conditions result in vector stocks with P:I ratios of <50. An efficient and reproducible protocol based on partial purification of an initial freeze and thaw lysate followed by chromatography for the purification and concentration of rAAV1 vectors has been developed for AAV1 clinical manufacturing.

To date, six rAAV vectors have been tested in humans: rAAV-CFTR, rAAV-factor IX, rAAV-sarcoglycan, rAAV-aspartoacylase, rAAV-alpha-1 antitrypsin and rAAV-microdystrophin. Extensive work has been done on the rAAV-factor IX vector by a consortium of investigators from the Children's Hospital of Pennsylvania, Stanford University and Avigen. The rAAV-factor IX vector was shown to be capable of long-term correction of the coagulopathy in both the factor IX-deficient mouse and the hemophilia B dog model. Intramuscular administration and portal vein administration were both efficacious in the dog model. Intramuscular administration in the mouse model was associated with the development of a humoral immune response to factor IX, which appears to have been related to the adherence of factor IX to type IV collagen in the extracellular matrix of the muscle. A clinical trial of intramuscular administration was reported, in which some biological activity of the vector was noted at a low dose, without obvious toxicity. The trial for Canavan's disease (aspartoacyclase deficiency) has been completed without adverse events. Currently, the trial of AAV-1 expressing alph-1 antitrypsin has been completed, with no adverse events reported. Additionally, enrollment is completed for ten subjects in Cohorts I-IV of the “Phase I Trial of Ocular Sub-Retinal Injection of a Recombinant Adeno-Associated Virus (rAAV-RPE65) Gene Vector in Patients with Retinal Disease Due to RPE65 Mutations.” There are many similarities in the RPE65 study to the study and technology here, where a surgical route of delivery is used to reach the target tissue. There is evidence of safety at two dose levels as well as indication of restoration of retinal function and improved vision in that study.

III. Tolerance-Inducing Gene Therapy Vector-Based Compositions and Methods for Modulating Immune Response to a Therapeutic rAAV.

Described are non-limiting embodiments of tolerance-inducing gene therapy vectors and methods for use in modulating immune response in a subject. In some embodiments, modulating immune response comprises inducing immune tolerance to an rAAV vector, such as an rAAV vector encoding a therapeutic nucleic acid. In some embodiments, the compositions and methods utilize a tolerance inducing gene therapy vector and optionally a therapeutic rAAV. In some embodiments, therapeutic rAAV encodes an antigen and administration of the therapeutic rAAV to the subject induces an immune response against an antigen. Inducing an immune response can be used to prevent or reduce adverse health impacts due to infection or cancer. In some embodiments, therapeutic rAAV encodes a therapeutic protein or RNA. Expression of the therapeutic protein or RNA can treat a disease or condition or one or more symptoms associated with a disease or condition.

a. Tolerance-Inducing Gene Therapy Vector

A tolerance-inducing gene therapy vector comprises a liver-targeted or HSC-targeted gene therapy vector comprising a nucleic acid sequence encoding at least an immunogenic portion of a capsid protein of the therapeutic rAAV. The tolerance-inducing gene therapy vector is capable of infecting or transfecting a liver cell or HSC such that the nucleic acid encoding the immunogenic portion of the capsid protein is expressed in the liver or HSC thereby inducing immune tolerance to the therapeutic rAAV. The nucleic acid sequence encoding the immunogenic portion of a capsid protein is operably linked to a promoter. The tolerance-inducing gene therapy vector can comprise a non-viral vector or a viral vector.

The tolerance-inducing gene therapy vector is configured to express an immunogenic peptide that is derived from the therapeutic rAAV. In some embodiments, the tolerance-inducing gene therapy vector is configured to express the immunogenic peptide specifically in the liver of the subject. Liver-specific expression may be facilitated by a liver-specific promoter. Liver-specific expression of an immunogenic portion of a capsid protein of the therapeutic rAAV results in the liver of the subject processing the immunogenic portion of the capsid protein, thereby inducing immune tolerance to that protein, thereby reducing, eliminating, or preventing an immune response to the therapeutic rAAV vector. As an example, liver-specific expression of an immunogenic portion of the AAV1 VP3 capsid protein results in the liver of the subject processing the VP3 capsid protein thereby inducing immune tolerance to the AAV1 VP3 protein, thereby reducing, eliminating, or preventing an immune response to an rAAV serotype 1 vector. Inducing tolerance to the therapeutic rAAV facilitates multiple administrations of the therapeutic rAAV1 vector to the subject. Multiple administrations may be used to deliver the same or different transgenes to the subject at different times. For example, multiple administrations of a transgene encoding a viral antigen may facilitate prime and booster injections or may facilitate re-design and repeat vaccination with viral antigen variants that result from mutations. Alternatively, multiple administrations of the therapeutic rAAV may be used to provide for treatment of a condition that requires repeat or continued dosing.

The tolerance-inducing gene therapy vector is configured to express an immunogenic peptide that is derived from the therapeutic rAAV. In addition to expression in liver, bone marrow chimerism can also lead to tolerance induction to an AAV capsid. In some embodiments, the tolerance-inducing gene therapy vector is configured to express the immunogenic peptide in HSC. The tolerance-inducing gene therapy vectors can be used to deliver a nucleic acid encoding the immunogenic peptide to HSC in vivo or ex vivo. For delivery to HSC ex vivo, the HSC expressing the immunogenic peptide are administered to the subject after transfection by the vector. HSC-specific expression may be facilitated by a HSC-specific promoter. Expression of the immunogenic portion of a capsid protein of the therapeutic rAAV in HSC in the subject can induce immune tolerance to the protein, thereby reducing, eliminating, or preventing an immune response to the therapeutic rAAV vector. Inducing tolerance to the therapeutic rAAV facilitates multiple administrations of the therapeutic rAAV1 vector to the subject. Multiple administrations may be used to deliver the same or different transgenes to the subject at different times. For example, multiple administrations of a transgene encoding a viral antigen may facilitate prime and booster injections or may facilitate re-design and repeat vaccination with viral antigen variants that result from mutations. Alternatively, multiple administrations of the therapeutic rAAV may be used to provide for treatment of a condition that requires repeat or continued dosing.

A non-viral tolerance-inducing gene therapy vector can comprise a lipoplex, polyplex, a lipopolyplex, a polymersome, or a nanoparticle. In some embodiments, the gene therapy vector comprises a lipid nanoparticle (LNP).

In some embodiments, the tolerance-inducing gene therapy vector comprises a lentiviral vector. A tolerance-inducing lentiviral vector can be any lentiviral vector that is capable of infection a liver cell or an HSC such that the nucleic acid encoding the immunogenic portion of the capsid protein is expressed in the liver cell or HSC.

In some embodiments, the nucleic acid sequence encoding at least an immunogenic portion of a capsid protein of the therapeutic rAAV is operatively linked to a promoter. The promoter drives expression of the immunogenic portion of a capsid protein. The promoter can be, but is not limited to, a constitutive promoter, an inducible promoter, a liver-specific promoter, a hepatocyte-specific promoter, a HSC-specific promoter or a synthetic promoter. In some embodiments, the promoter is a liver-specific promoter. A constitutive promoter can be, but is not limited to, a Herpes Simplex virus (HSV) promoter, a thymidine kinase (TK) promoter, a Rous Sarcoma Virus (RSV) promoter, a Simian Virus 40 (SV40) promoter, a Mouse Mammary Tumor Virus (MMTV) promoter, an Adenovirus E1A promoter, a cytomegalovirus (CMV) promoter, a mammalian housekeeping gene promoter, an EF1 promoter, or a β-actin promoter. An inducible promoter can be, but is not limited to, a cytochrome P450 gene promoter, a heat shock protein gene promoter, a metallothionein gene promoter, a hormone-inducible gene promoter, an estrogen gene promoter, or a tetVP16 promoter that is responsive to tetracycline. A liver-specific promoter can be, but is not limited to, an albumin promoter, an alpha-1-antitrypsin promoter, or a hepatitis B virus core protein promoter. In some embodiments, the nucleic acid sequence encoding the at least an immunogenic portion of a capsid protein of the therapeutic rAAV is operatively linked to a liver-specific promoter that results in expression of the at least a portion of a capsid protein solely or substantially in the liver.

In some embodiments, the nucleic acid sequence encoding the at least an immunogenic portion of a capsid protein of the therapeutic rAAV is operatively linked to a synthetic promoter comprising the nucleic acid sequence of SEQ ID NO: 3, or a nucleic acid sequence having at least 90% sequence identity to SEQ ID NO: 3.

In some embodiments, the immunogenic portion of the capsid protein of a therapeutic rAAV comprises all or an immunogenic portion of a capsid protein of the therapeutic rAAV. The immunogenic portion of the capsid protein can comprise at least 10% at least 20%, at least 30%, at least 40%, at least 50%, at least 60%, at least 70%, at least 80%, at least 90% of the capsid protein. In some embodiments, the immunogenic portion of the capsid protein comprises an immunogenic portion of one or more of: a VP1 capsid protein, a VP2 capsid protein, and a VP3 capsid protein. In some embodiments, the immunogenic portion of the capsid protein comprises an immunogenic portion of the VP3 capsid protein. In some embodiments, the immunogenic portion of the capsid protein has at least 90% identical, at least 95% identical, or 100% identical to the amino acid sequence of SEQ ID NO: 1. In some embodiments, the immunogenic portion of the capsid protein is encoded by a nucleic acid sequence having at least 90% identify, at least 95% identity, or 100% identity to SEQ ID NO: 2.

The tolerance-inducing gene therapy vectors deliver the nucleic acid encoding the immunogenic portion of the capsid protein of a therapeutic rAAV to a liver cell or HSC, resulting in production of the immunogenic portion of the capsid protein in the liver or in HSC. The immunogenic portion of the capsid protein may be secreted or it may remain associated with the cell in which it is expressed. The tolerance-inducing gene therapy vector induces specific immune tolerance to the therapeutic rAAV in the subject.

### IV. LNPs

The present disclosure provides additional embodiments for delivering viral antigens, e.g., AAV capsids proteins or nucleic acids encoding the AAV capsid proteins to the liver of a subject using lipid nanoparticle (LNP) compositions. The LNP may contain (i) a cationic lipid, (ii) a neutral lipid, (iii) a helper lipid (e.g., a sterol), (iv) a stealth lipid (e.g., a PEG lipid), and/or combinations of any of (i) through (iv). In certain embodiments, the LNP cargo includes a nucleic acid (e.g., an mRNA) encoding viral antigens, e.g., AAV capsid proteins or portions thereof.

### A. LNP Formulations

Disclosed herein are various embodiments of LNP formulations, e.g., for delivering viral antigen sequences or nucleic acids encoding the viral antigens to the liver. The LNP formulations comprise a cationic lipid. In some embodiments, the cationic lipid is cationic at certain pH levels, e.g., cationic in an acidic environment, such as in the lysosome of a target cell. In some embodiments, the LNP further includes one or more of a helper lipid, a neutral lipid, and a stealth lipid. In some embodiments the LNP formulation forms microspheres, including unilamellar and multilamellar vesicles, which may also be referred to as “liposomes”. In some embodiments the microspheres comprise lamellar phase lipid bilayers that, in some embodiments, are substantially spherical and can comprise an aqueous core, e.g., comprising a substantial portion of mRNA molecules.

The LNP compositions provided herein are preferentially taken up by liver cells (e.g., hepatocytes). In some embodiments, the LNP compositions bind to apolipoproteins such as apolipoprotein E (ApoE) in the blood. Apolipoproteins are proteins circulating in plasma that are key in regulating lipid transport. ApoE represents one class of apolipoproteins which interacts with cell surface heparin sulfate proteoglycans in the liver during the uptake of lipoprotein. (See e.g., Scherphof and Kamps, The role of hepatocytes in the clearance of liposomes from the blood circulation. Prog Lipid Res. 2001 May;40 (3): 149-66).

### B. Cationic Lipids

Lipid compositions comprising LNPs for delivery of viral antigen sequences provided herein, e.g., to a liver cell, comprise a cationic lipid. As further described herein, the cationic lipids of the present disclosure may be ionizable depending upon the pH of the medium they are in. For example, in a slightly acidic medium, the cationic lipids may be protonated and thus bear a positive charge. Conversely, in a slightly basic medium, such as, for example, blood where pH is approximately 7.35, the cationic lipids may not be protonated and thus bear no charge. The ability of a cationic lipid to bear a charge is related to its intrinsic pKa. For example, the cationic lipids of the present disclosure may each, independently, have a pKa in the range of from about 5.8 to about 6.2. This may be advantageous as it has been found that cationic lipids with a pKa ranging from about 5.1 to about 7.4 are effective for delivery of cargo to the liver.

In some embodiments, the cationic lipid is a low molecular weight cationic lipid such as those described in U.S. patent application No. 20130090372, the contents of which are herein incorporated by reference in their entirety. The cationic lipid can be, but is not limited to, a cationic fatty acid, a cationic glycerolipid, a cationic glycerophospholipid, a cationic sphingolipid, a cationic sterol lipid, a cationic prenol lipid, a cationic saccharolipid, or a cationic polyketide. In certain embodiments, the cationic lipid comprises two fatty acyl chains, each chain of which is independently saturated or unsaturated. In some embodiments, the cationic lipid is a diglyceride. For example, in some instances, the cationic lipid may be a cationic lipid of Formula I or Formula II:

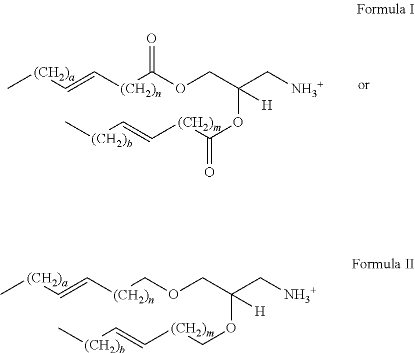

wherein each of a, b, n, and m is independently an integer between 2 and 12 (e.g., between 3 and 10). In some embodiments, the cationic lipid is a cationic lipid of Formula I wherein each of a, b, n, and m is independently an integer selected from 3, 4, 5, 6, 7, 8, 9, and 10. In certain embodiments, the cationic lipid is DOTAP (1,2-dioleoyl-3-trimethylammonium-propane), or a derivative thereof. In other embodiments, the cationic lipid is DOTMA (1,2-di-0-octadecenyl-3-trimethylammonium propane), or a derivative thereof.

In some embodiments, the LNPs comprise liposomes formed from 1,2-dioleyloxy-N,N-dimethylaminopropane (DODMA) liposomes, Dil_a2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (See e.g., US20100324120; herein incorporated by reference in its entirety). In some embodiments, the LNPs comprise liposomes formed from the synthesis of stabilized plasmid-lipid particles (SPLP) or stabilized nucleic acid lipid particle (SNALP) that have been previously described and shown to be suitable for oligonucleotide delivery in vitro and in vivo.

In some embodiments, the cationic lipid is 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}-propan-1-ol (Compound 2 in US20130150625); 2-amino-3-[(9Z,12Z)-octadeca-9,12-dien-1-yloxy]-2-[(octyloxy)methyl]propan-1-ol (Compound 3 in U.S. 20130150625); 2-(dimethylamino)-3-[(9Z,12Z)-octadeca-9,12-dien-1-yloxy]-2-{[(9Z,12Z)-octadeca-9,12-dien-1-yloxy]methyl}-propan-1-ol (Compound 4 in US20130150625); or any pharmaceutically acceptable salt or stereoisomer of any of the forgoing.

In some embodiments, the cationic lipid is (9Z,12Z)-3-((4,4-bis(octyloxy)-butanoyl)oxy)-2-((((3-(diethylamino)propoxy)carbonyl)oxy)methyl)propyloctadeca-9,12-dienoate, also called 3-((4,4-bis(octyloxy) butanoyl)oxy)-2-((((3-(diethylamino)propoxy)-carbonyl)oxy)methyl) propyl (9Z,12Z)-octadeca-9,12-dienoate (“Lipid A”, as further described in WO2017173054). In some embodiments, the cationic lipid is ((5-((dimethylamino)methyl)-1,3-phenylene)bis(oxy))bis(octane-8,1-diyl)bis(decanoate), also called ((5-((dimethylamino)-methyl)-1,3-phenylene)bis(oxy))bis(octane-8,1-diyl)bis(decanoate) (“Lipid B”, as further described in WO2017173054). In some embodiments, the cationic lipid is 2-((4-(((3-(dimethylamino)-propoxy)carbonyl)oxy)hexadecanoyl)oxy)propane-1,3-diyl(9Z,9′Z,12Z,12′Z)-bis(octadeca-9,12-dienoate) (“Lipid C”, as further described in WO2017173054A1). In some embodiments, the cationic lipid is 3-(((3-(dimethylamino)propoxy)carbonyl)oxy)-13-(octanoyl-oxy)tridecyl-3-octylundecanoate (“Lipid D”, as further described in WO2017173054).

### C. Additional Components and Formulations

In some embodiments, the LNP further comprises other lipid components, such as neutral lipids, helper lipids, and stealth lipids. “Neutral lipids” suitable for use in a lipid composition of the disclosure include, for example, a variety of neutral, uncharged or zwitterionic lipids. Examples of neutral phospholipids suitable for use in the present disclosure include, at are not limited to, 5-heptadecylbenzene-1,3-diol (resorcinol), dipalmitoylphosphatidylcholine (DPPC), distearoylphosphatidylcholine (DSPC), phosphocholine (DOPC), dimyristoyl-phosphatidylcholine (DMPC), phosphatidylcholine (PLPC), 1,2-distearoyl-sn-glycero-3-phospho-choline (DAPC), phosphatidylethanolamine (PE), egg phosphatidylcholine (EPC), dilauryloyl-phosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphor-choline (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidyl-ethanolamine (DSPE), dimyristoyl phosphatidylethanolamine (DMPE), dipalmitoyl phosphatidyl-ethanolamine (DPPE), palmitoyloleoyl phosphatidylethanolamine (POPE), lysophosphatidyl-ethanolamine and combinations thereof. In some embodiments, the neutral phospholipid is selected from the group consisting of distearoylphosphatidylcholine (DSPC) and dimyristoyl phosphatidyl ethanolamine (DMPE). In another embodiment, the neutral phospholipid is distearoylphosphatidylcholine (DSPC). Neutral lipids function to stabilize and improve processing of the LNPs.

“Helper lipids” are lipids that enhance transfection (e.g., transfection of the LNP including the biologically active agent). The mechanism by which the helper lipid enhances transfection includes enhancing particle stability. In certain embodiments, the helper lipid enhances membrane fusogenicity. Helper lipids include steroids, sterols, and alkyl resorcinols. Helper lipids suitable for use in the present disclosure include, but are not limited to, cholesterol, 5-heptadecylresorcinol, and cholesterol hemisuccinate. In some embodiments, the helper is cholesterol. In some embodiments, the helper lipid is cholesterol hemisuccinate.

“Stealth lipids” are lipids that, e.g., alter the length of time the nanoparticles can exist in vivo (e.g., in the blood). Stealth lipids may assist in the formulation process by, for example, reducing particle aggregation and controlling particle size. Stealth lipids used herein may modulate pharmacokinetic properties of the LNP. Stealth lipids suitable for use in a lipid composition of the disclosure include, but are not limited to, stealth lipids having a hydrophilic head group linked to a lipid moiety. Stealth lipids suitable for use in a lipid composition of the present disclosure and information about the biochemistry of such lipids can be found in Romberg et al, Pharmaceutical Research, Vol. 25, No. 1, 2008, pg. 55-71 and Hoekstra et al, Biochimica et Biophysica Acta 1660 (2004) 41-52. Additional suitable PEG lipids are disclosed, e.g., in WO2006007712.

In some embodiments, the hydrophilic head group of stealth lipid comprises a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids and poly N-(2-hydroxypropyl) methacrylamide.

Stealth lipids may comprise a lipid moiety. The lipid moiety of the stealth lipid may be derived from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. The dialkylglycerol or dialkylglycamide group can further comprise one or more substituted alkyl groups.

Unless otherwise indicated, the term “PEG” as used herein means any polyethylene glycol or other polyalkylene ether polymer. In some embodiments, PEG is an optionally substituted linear or branched polymer of ethylene glycol or ethylene oxide. In some embodiments, PEG is unsubstituted. In some embodiments, the PEG is substituted, e.g., by one or more alkyl, alkoxy, acyl, hydroxy, or aryl groups. In some embodiments, the PEG has a molecular weight of from about 130 to about 50,000; of from about 150 to about 30,000; of from about 150 to about 20,000; of from about 150 to about 15,000; of from about 150 to about 10,000; of from about 150 to about 6,000; of from about 150 to about 5,000; of from about 150 to about 4,000; of from about 150 to about 3,000; of from about 300 to about 3,000; of from about 1,000 to about 3,000; or from about 1,500 to about 2,500.

In certain embodiments, the PEG (e.g., conjugated to a lipid, such as a stealth lipid), is a “PEG-2K,” also termed “PEG 2000,” which has an average molecular weight of about 2,000 daltons.

In any of the embodiments described herein, the stealth lipid may be selected from PEG-dilauroylglycerol, PEG-dimyristoylglycerol (PEG-DMG) (catalog #GM-020 from NOF, Tokyo, Japan), PEG-dipalmitoylglycerol, PEG-distearoylglycerol (PEG-DSPE) (catalog #DSPE-020CN, NOF, Tokyo, Japan), PEG-dilaurylglycamide, PEG-dimyristylglycamide, PEG-dipalmitoylglycamide, PEG-distearoylglycamide, PEG-cholesterol (1-[8′-(Cholest-5-en-3 [beta]-oxy) carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol) ether), 1,2-dimyristoyl-sn-glycero-3-phosphoethanolamine-N-[methoxy (polyethylene glycol)-2000](PEG2k-DMG) (cat. #880150P from Avanti Polar Lipids, Alabaster, Alabama, USA), 1,2-distearoyl-sn-glycero-3-phosphoethanolamine-N-[methoxy (polyethylene glycol)-2000] (PEG2k-DSPE) (cat. #880120C from Avanti Polar Lipids, Alabaster, Alabama, USA), 1,2-distearoyl-sn-glycerol, methoxypoly ethylene glycol (PEG2k-DSG; GS-020, NOF Tokyo, Japan), poly(ethylene glycol)-2000-dimethacrylate (PEG2k-DMA), and 1,2-distearyloxypropyl-3-amine-N-[methoxy (polyethylene glycol)-2000] (PEG2k-DSA). In some embodiments, the stealth lipid is PEG2k-DMG. In some embodiments, the stealth lipid is PEG2k-DSG. In some embodiments, the stealth lipid is PEG2k-DSPE. In some embodiments, the stealth lipid is PEG2k-DMA. In some embodiments, the stealth lipid is PEG2k-DSA. In some embodiments, the stealth lipid is PEG2k-Cl 1. In some embodiments, the stealth lipid is PEG2k-C14. In some embodiments, the stealth lipid is PEG2k-C16. In some embodiments, the stealth lipid is PEG2k-C18.

In some embodiments, an LNP composition comprises a cationic lipid and an mRNA encoding a viral antigen, such as an AAV capsid protein (or portion thereof). In some embodiments, an LNP composition comprises a cationic lipid, an mRNA encoding a viral antigen, and at least one other lipid component chosen from a helper lipid, a neutral lipid, or a stealth lipid. In certain compositions, the helper lipid is cholesterol. In some compositions, the neutral lipid is DSPC. In additional embodiments, the stealth lipid is PEG2k-DMG. In some embodiments, an LNP composition comprises a cationic lipid, a helper lipid, a neutral lipid, a stealth lipid, and an mRNA encoding a viral antigen (as provided herein).

Certain embodiments also provide LNP compositions described according to the respective molar ratios of the component lipids in the formulation. In some embodiments, the mol-% of the cationic lipid is from about 30 mol-% to about 60 mol-%. In some embodiments, the mol-% of the cationic lipid is from about 35 mol-% to about 55 mol-%. In some embodiments, the mol-% of the cationic lipid is from about 40 mol-% to about 50 mol-%. In some embodiments, the mol-% of the cationic lipid is from about 42 mol-% to about 47 mol-%. In some embodiments, the mol-%) of the cationic lipid is about 45%. In some embodiments, the cationic lipid mol-% of the L P batch is ±30%, ±25%, ±20%, ±15%, ±10%, ±5%, or ±2.5% of the target mol-%. In some embodiments, LNP inter-lot variability is less than 15%, less than 10% or less than 5%.

In some embodiments, the mol-% of the helper lipid is from about 30 mol-% to about 60 mol-%. In some embodiments, the mol-% of the helper lipid is from about 35 mol-% to about 55 mol-%. In some embodiments, the mol-% of the helper lipid is from about 40 mol-% to about 50 mol-%. In some embodiments, the mol-% of the helper lipid is from about 41 mol-% to about 46 mol-%. In some embodiments, the mol-% of the helper lipid is about 44 mol-%. In some embodiments, the helper mol-% of the LNP batch is ±30%, ±25%, ±20%, ±15%, ±10%, ±5%, or ±2.5% of the target mol-%. In certain embodiments, LNP inter- lot variability is less than 15%, less than 10% or less than 5%.

In some embodiments, the mol-% of the neutral lipid is from about 1 mol-% to about 20 mol-%. In some embodiments, the mol-% of the neutral lipid is from about 5 mol-% to about 15 mol-%. In some embodiments, the mol-% of the neutral lipid is from about 7 mol-% to about 12 mol-%. In some embodiments, the mol-% of the neutral lipid is about 9 mol-%. In some embodiments, the neutral lipid mol-% of the LNP batch is ±30%, ±25%, ±20%, ±15%, ±10%, ±5%, or ±2.5% of the target mol-%. In certain embodiments, LNP inter-lot variability is less than 15%, less than 10% or less than 5%.

In some embodiments, the mol-% of the stealth lipid is from about 1 mol-% to about 10 mol-%. In some embodiments, the mol-% of the stealth lipid is from about 1 mol-% to about 5 mol-%. In some embodiments, the mol-% of the stealth lipid is from about 1 mol-% to about 3 mol-%. In some embodiments, the mol-%> of the stealth lipid is about 2 mol-%. In some embodiments, the mol-% of the stealth lipid is about 1 mol-%. In some embodiments, the stealth lipid mol-% of the LNP batch will be ±30%, ±25%, ±20%, ±15%, ±10%, ±5%, or ±2.5% of the target mol-%. In certain embodiments, LNP inter-lot variability will be less than 15%, less than 10% or less than 5%.

Embodiments of the present disclosure also provide lipid compositions described according to the ratio between the positively charged amine groups of the cationic lipid (N) and the negatively charged phosphate groups (P) of the nucleic acid to be encapsulated. This may be mathematically represented by the equation N/P. In some embodiments, the N/P ratio is from about 0.5 to about 100. In some embodiments, the N/P ratio is from about 1 to about 50. In some embodiments, the N/P ratio is from about 1 to about 25. In some embodiments, the N/P ratio is from about 1 to about 10. In some embodiments, the N/P ratio is from about 1 to about 7. In some embodiments, the N/P ratio is from about 3 to about 5. In some embodiments, the N/P ratio is from about 4 to about 5. In some embodiments, the N/P ratio is about 4. In some embodiments, the N/P ratio is about 4.5. In some embodiments, the N/P ratio is about 5.

### D. LNP Cargo

In some embodiments, the cargo component of the disclosed LNP formulation comprises a nucleic acid, e.g., a DNA or RNA encoding at least a portion of a viral antigen, e.g., at least a portion of a AAV capsid protein. In some embodiments, the mRNA is modified for improved stability and/or immunogenicity properties. The modifications may be made to one or more nucleosides within the mRNA. Examples of chemical modifications to mRNA nucleobases include pseudouridine, 1-methyl-pseudouridine, and 5-methyl-cytidine. The mRNA encoding the viral antigen may be codon optimized for expression in a particular tissue and/or cell type, such as the liver and/or hepatocytes. In some embodiments, the mRNA encodes a human codon optimized AAV capsid protein (e.g., a VP1 protein sequence or portion thereof).

In addition to the coding sequence for a viral antigen, an mRNA may comprise a 3′ and/or 5′ untranslated region (UTR). In some embodiments, the 3′ or 5′ UTR can be derived from a human or viral gene sequence, such as those that are highly expressed in the liver. Exemplary 3′ and 5′ human gene UTRs include α- and β-globin, albumin, HSD17B4, and eukaryotic elongation factor 1a. Exemplary viral-derived 5′ and 3′ UTRs include orthopoxvirus and cytomegalovirus UTR sequences.

In certain embodiments, an mRNA includes a 5′ cap, such as m⁷G (5′)ppp(5′)N. In some embodiments, the cap is (i) a cap-0 structure where nucleotide N does not contain 2′-OMe; (ii) a cap-1 structure where nucleotide N contains 2′-OMe; or (iii) a cap-2 structure where nucleotides N and N+1 contain 2′-OMe. In some embodiments, the cap comprises an anti-reverse-cap analog (ARCA) structure.

In certain embodiments, an mRNA includes a poly(A) tail. This tail may be about 40 to about 300 nucleotides in length. In some embodiments, the tail is about 40 to about 100 nucleotides in length. In some embodiments, the tail is about 100 to about 300 nucleotides in length. In some embodiments, the tail is about 100 to about 200 nucleotides in length. In some embodiments, the tail is about 50 to about 200 nucleotides in length. In some embodiments, the tail is about 50 to about 250 nucleotides in length. In certain embodiments, the tail is about 100, about 150, or about 200 nucleotides in length.

In some embodiments, the mRNA is purified. In some embodiments, the mRNA is purified using a precipitation method (e.g., LiCl precipitation, alcohol precipitation, or an equivalent method, e.g., as described herein). In some embodiments, the mRNA is purified using a chromatography-based method, such as an HPLC-based method. In some embodiments, the mRNA is purified using both a precipitation method (e.g., LiCl precipitation) and an HPLC-based method.

V. Therapeutic rAAV

A therapeutic rAAV comprises a nucleotide sequence encoding a therapeutic gene (e.g., protein or RNA) and is able to transfect (transduce) a cell in the subject, resulting in expression of the therapeutic gene in the cell of the subject. In some embodiments, the therapeutic rAAV preferentially infects neuronal or skeletal muscle cells.

The therapeutic nucleic acid can encode an expressible gene. The expressible gene can be, but is not limited to, a therapeutic RNA or a therapeutic polypeptide. A therapeutic RNA can be, but is not limited to, a mRNA, an aptamer, a microRNA, an siRNA, an RNA interference polynucleotide, an antisense RNA, a ribozyme, or an RNA from a CRISPR/Cas system (e.g., a Class 1 or Class 2 Cas, such as Cas3, Cas8a, Cas5, Cas8b, Cas8c, Cas10d, Cse1, Cse2, Csy1, Csy2, Csy3, GSU0054, Cas10, Csm2, Cmr5, Cas10, Csx11, Csx10, Csf1, Cas9, Csn2, Cas4, Cpf1, C2c1, C2c3, Cas13a (previously known as C2c2), Cas13b, Cas13c, CasX, or CasY). The therapeutic polypeptide can be, but is not limited to, an antigen, an antibody, and immunoglobulin, an antigen-binding molecule, an antigen binding fragment of an antibody, a protein, a functional protein that replaces an abnormal or non-functional protein in the subject (or a functional fragment thereof), or an anti-tumor protein such as a tumor suppressor or an immune activating protein. The antigen may be an antigen of a pathogen and a tumor antigen. The pathogen can be, but is not limited to a viral antigen, a bacterial antigen, a parasite antigen, a fungal antigen. The therapeutic polypeptide can be, but is not limited to, human frataxin, myotubularin, a muscle protein, a neuronal protein, acid alfa glucosidase (GAA), and aspartoacylase.

In some embodiments, the therapeutic rAAV has a sequence sharing at least 85% sequence identity to SEQ ID NO: 6 and/or SEQ ID NO: 8. In some embodiments, the therapeutic rAAV has a sequence sharing at least 95% sequence identity to SEQ ID NO: 6 and/or SEQ ID NO: 8. In some embodiments, the viral antigen is at least a portion of a SARS-CoV-2 polypeptide. In some embodiments, the SARS-COV-2 polypeptide comprises an S1 subunit of the SARS-COV-2 spike glycoprotein. In some embodiments, the SARS-Cov-2 polypeptide comprises an N-terminal or C-terminal domain of an SI subunit of the SARS-Cov-2 spike glycoprotein. In some embodiments, the viral protein is derived from the flu virus, HIV, or other virus.

The promoter driving expression of the therapeutic nucleic acid can be, but is not limited to, a constitutive promoter, an inducible promoter, a tissue-specific promoter, a neuronal-specific promoter, a muscle-specific promoter, or a synthetic promoter. In some embodiments, the promoter is a neuronal-specific promoter or a muscle-specific promoter. A constitutive promoter can be, but is not limited to, a Herpes Simplex virus (HSV) promoter, a thymidine kinase (TK) promoter, a Rous Sarcoma Virus (RSV) promoter, a Simian Virus 40 (SV40) promoter, a Mouse Mammary Tumor Virus (MMTV) promoter, an Adenovirus E1A promoter, a cytomegalovirus (CMV) promoter, a mammalian housekeeping gene promoter, or a β-actin promoter. An inducible promoter can be, but is not limited to, a cytochrome P450 gene promoter, a heat shock protein gene promoter, a metallothionein gene promoter, a hormone-inducible gene promoter, an estrogen gene promoter, or a tetVP16 promoter that is responsive to tetracycline. A muscle-specific promoter can be, but is not limited to, desmin promoter, a creatine kinase promoter, a myogenin promoter, an alpha myosin heavy chain promoter, or a natriuretic peptide promoter.

In some embodiments, the tolerance-inducing gene therapy vector comprises a liver-specific promoter and the therapeutic rAAV promoter comprises a tissue-specific promoter. The tissue-specific promoter can be, but is not limited to, a neuronal-or muscle-specific promoter.

In some embodiments, the tolerance-inducing gene therapy vector comprises a HSC-specific promoter and the therapeutic rAAV promoter comprises a tissue-specific promoter. The tissue-specific promoter can be, but is not limited to, a neuronal-or muscle-specific promoter.

The therapeutic rAAV can be serotype 1, serotype 2, serotype 3, serotype 4, serotype 5, serotype 6, serotype 7, serotype 8, serotype 9, serotype 10, serotype 11, serotype 12, rh10, or rh74. The therapeutic rAAV can also be a pseudo-type rAAV. For a pseudo-type therapeutic rAAV, the serotype of the capsid protein is different from the serotype of the capsid protein of the tolerance inducing rAAV. In some embodiments, the therapeutic rAAV is serotype 1.

In some embodiments, therapeutic rAAV is serotype 1 AAV (AAV1) and the tolerance inducing rAAV is serotype 2 AAV (AAV2). In some embodiments, therapeutic rAAV is serotype 1 AAV (AAV1) and the tolerance inducing rAAV is serotype 9 AAV (AAV9). In some embodiments, therapeutic rAAV is serotype 1 AAV (AAV1) and the tolerance inducing rAAV is rh74.

In some embodiments, the tolerance-inducing gene therapy vector is a non-viral vector and the therapeutic rAAV can be of any serotype.

In some embodiments, the tolerance-inducing gene therapy vector is a lentiviral vector, and the therapeutic rAAV can be of any serotype.

### VI. Promoter

The tolerance-inducing gene therapy vector and the therapeutic rAAV vector each comprise a nucleic acid sequence encoding an expressible gene operatively linked to an expression control sequence. The expression control sequence facilitates expression of the expressible gene. In some embodiments, the expression control sequence is heterologous to the expressible gene. Numerous expression control sequences are known in the art. Non-limiting examples of expression control sequences include, but are not limited to, promoters, insulators, silencers, response elements, introns, enhancers, initiation sites, termination signals, and poly(A) tails. Any combination of such control sequences is contemplated herein (e.g., a promoter and an enhancer).

To achieve appropriate expression levels of the nucleic acid, protein, or polypeptide of interest, any of a number of promoters suitable for use in the selected host cell may be employed. The promoter may be, for example, a constitutive promoter, tissue-specific promoter, inducible promoter, or a synthetic promoter. For example, constitutive promoters of different strengths can be used. A tolerance-inducing gene therapy vector or a therapeutic rAAV described herein may include one or more constitutive promoters, such as viral promoters or promoters from mammalian genes that are generally active in promoting transcription. Non-limiting examples of constitutive viral promoters include the Herpes Simplex virus (HSV), thymidine kinase (TK), Rous Sarcoma Virus (RSV), Simian Virus 40 (SV40), Mouse Mammary Tumor Virus (MMTV), Ad E1A and cytomegalovirus (CMV) promoters. Non-limiting examples of constitutive mammalian promoters include various housekeeping gene promoters, as exemplified by the β-actin promoter.

Inducible promoters and/or regulatory elements may also be contemplated for achieving appropriate expression levels of the protein or polypeptide of interest. Non-limiting examples of suitable inducible promoters include those from genes such as cytochrome P450 genes, heat shock protein genes, metallothionein genes, and hormone-inducible genes, such as the estrogen gene promoter. Another example of an inducible promoter is the tetVP16 promoter that is responsive to tetracycline.

Tissue-specific promoters and/or regulatory elements are also contemplated herein. Non-limiting examples of such promoters that may be used include (1) desmin, creatine kinase, myogenin, alpha myosin heavy chain, and natriuretic peptide, specific for muscle cells, and (2) albumin, alpha-1-antitrypsin, hepatitis B virus core protein promoters, specific for liver cells.

Synthetic promoters are also contemplated herein. A synthetic promoter may comprise, for example, regions of known promoters, regulatory elements, transcription factor binding sites, enhancer elements, repressor elements, and the like. In some embodiments, a synthetic promoter comprises the nucleic acid sequence of SEQ ID NO: 3 or a nucleic acid sequence having at least 90%, at least 95%, at least 96%, at least 97%, or at least 98% sequence identity to SEQ ID NO: 3.

### VII. Inducing Tolerance

It is desirable to reduce, eliminate, or prevent an immune response to a therapeutic rAAV to facilitate expression of a therapeutic nucleic acid delivered by the therapeutic rAAV. In some embodiments, reducing, eliminating, or preventing immune response to a therapeutic rAAV vector comprises reducing antibodies or T cells, such a Teff cells, specific to the therapeutic AAV in the subject. In some embodiments, reducing, eliminating, or preventing an immune response to a therapeutic rAAV vector provides for repeat administration of the therapeutic rAAV. Reducing, eliminating, or preventing an immune response to the therapeutic rAAV vector facilitates administering the therapeutic rAAV to a subject two or more times. Administration of a therapeutic rAAV to a subject two or more times can be done to deliver the same therapeutic nucleic acid two or more times or to deliver different therapeutic nucleic acids using the same serotype rAAV.

The tolerance-inducing gene therapy vector and the therapeutic rAAV can be co-administered to a subject or administered sequentially. In some embodiments, the tolerance-inducing gene therapy vector is administered to a subject prior to administering the therapeutic rAAV. In some embodiments, the tolerance-inducing gene therapy vector is administered to a subject concurrently with administering the therapeutic rAAV. In some embodiments, the tolerance-inducing gene therapy vector is administered to a subject after administering the therapeutic rAAV.

In some embodiments, the tolerance-inducing gene therapy vector is administered to a subject prior to administering a first dose of the therapeutic rAAV. In some embodiments, the tolerance-inducing gene therapy vector is administered to a subject concurrently with administering a first dose of the therapeutic rAAV. In some embodiments, the tolerance-inducing gene therapy vector is administered to a subject after administering a first dose of the therapeutic rAAV.

In some embodiments, the tolerance-inducing gene therapy vector is administered to a subject prior to administering a second or subsequent dose of a therapeutic rAAV. In some embodiments, the tolerance-inducing gene therapy vector is administered to a subject concurrently with administering a second or subsequent dose of a therapeutic rAAV. In some embodiments, the tolerance-inducing gene therapy vector is administered to a subject after administering a second or subsequent dose of a therapeutic rAAV.

In some embodiments, the tolerance-inducing gene therapy vector is administered to a subject after administering a first dose of a therapeutic rAAV and prior to administering a second or subsequent dose of the therapeutic rAAV.

In some embodiments, the liver-specific promoter has at least 90% sequence identity to the sequence of SEQ ID NO: 3. In some embodiments, the liver-specific promoter comprises the sequence of SEQ ID NO: 3. In some embodiments, the immunogenic viral protein is a viral capsid protein. In some embodiments, the immunogenic viral protein comprises an immunogenic portion of one or more of the VP1, VP2, and/or VP3 capsid proteins. In some embodiments, the immunogenic viral protein is derived from the VP3 capsid protein. In some embodiments, the immunogenic viral protein is derived from an AAV serotype 1 VP3 capsid protein. In some embodiments, the immunogenic viral protein has at least 90% sequence identity to SEQ ID NO: 1. In some embodiments, the liver specific promoter drives expression of SEQ ID NO: 1, which is encoded by SEQ ID NO: 2. In some embodiments, the serotype of the rAAV vector that encodes SEQ ID NO: 2 is serotype 2. In some embodiments, the serotype of the rAAV vector that encodes SEQ ID NO: 2 is serotype 9. In some embodiments, the vector construct has at least 90% sequence identity to SEQ ID NO: 4.

In some embodiments, the therapeutic rAAV induces an immune response against an antigen in a subject and the tolerance-inducing gene therapy vector is used to induce specific immune tolerance to the therapeutic rAAV in the subject. The immune response can be, but is not limited to, a humoral immune response.

In some embodiments, the therapeutic rAAV induces an immune response against an tumor antigen in a subject and the tolerance-inducing gene therapy vector is used to induce specific immune tolerance to the therapeutic rAAV in the subject. The immune response can be, but is not limited to, a humoral immune response or a cellular immune response.

In some embodiments, the therapeutic rAAV provides a therapeutic gene for treatment of a disease or condition or for treatment of one or more symptoms associated with a disease or condition in a subject and the tolerance-inducing gene therapy vector is used to induce specific immune tolerance to the therapeutic rAAV in a subject.

Described are methods for inducing an immune response in a host comprising administering to the host an effective amount of a therapeutic rAAV of a first serotype comprising a nucleotide sequence encoding an antigen; and administering a tolerance-inducing gene therapy vector comprising a liver-specific promoter operatively linked to a nucleotide sequence encoding at least a portion of a capsid protein of the therapeutic rAAV, wherein the at least a portion of the capsid protein of the therapeutic rAAV is immunogenic, and wherein administration of the tolerance-inducing gene therapy vector results in inducing specific immune tolerance to the therapeutic rAAV in the host.

Described are methods for inducing an immune response in a host comprising administering to the host an effective amount of a therapeutic rAAV comprising a nucleotide sequence encoding an antigen; and administering a HSC-targeted tolerance-inducing gene therapy vector comprising a nucleotide sequence encoding at least a portion of a capsid protein of the therapeutic rAAV, wherein the at least a portion of the capsid protein of the therapeutic rAAV is immunogenic, and wherein administration of the tolerance-inducing gene therapy vector to the subject results in inducing specific immune tolerance to the therapeutic rAAV in the host.

Described are methods for inducing an immune response in a host comprising administering to the host an effective amount of a therapeutic rAAV comprising a nucleotide sequence encoding an antigen; and administering to the subject a effective dose of engineered HSC expressing a nucleotide sequence encoding at least a portion of a capsid protein of the therapeutic rAAV, wherein the at least a portion of the capsid protein of the therapeutic rAAV is immunogenic, and wherein effective dose of the engineered HSC to the subject results in inducing specific immune tolerance to the therapeutic rAAV in the host. The engineered HSC can be made by transfecting the cells with any of the described tolerance-inducing gene therapy vector.

In some embodiments, the therapeutic rAAV vector is a serotype 1 rAAV and is designed to express a transgene that is an antigenic peptide from a pathogen that could cause an unwanted infection in the subject. The pathogen can be, but is not limited to, an influenza virus or a coronavirus. In some embodiments, the therapeutic rAAV is configured to express the antigenic peptide from a pathogen in a muscle and/or a neuronal tissue. Expression of the antigenic peptide in the muscle or neuronal tissue can result in expression of the antigenic peptide and allow the subject's immune system to develop an immune (e.g., antibody) response against the antigenic peptide. The use of the tolerance-inducing gene therapy vector to induce a tolerance to the therapeutic rAAV vector allows repeat administration of the therapeutic rAAV vector. Repeat administration of the therapeutic rAAV enables vaccination against different pathogens, to provide vaccination to pathogen variants, or to provide booster vaccinations to the same pathogen. In some embodiments, the therapeutic rAAV vector drives expression of a SARS-COV-2 spike protein. In some embodiments, the spike protein has at least 90% sequence identity to SEQ ID NO: 5 and/or SEQ ID NO: 7. In some embodiments, the vector construct has at least 90% sequence identity to SEQ ID NO: 6 and/or SEQ ID NO: 8. In some embodiments, a cytomegalovirus promoter is used to drive the expression of the viral transgene. In some embodiments, the CMV promoter has at least 90% sequence identity to SEQ ID NO: 9.

The methods of the present disclosure can enhance the humoral immune response to a variety of pathogens or antigens thereof. Pathogens include, without limitation, one or more of the following: viruses, prions, parasites, fungi, mold, yeast, and bacteria (both). The virus can be, but is not limited to: a coronavirus, a human immunodeficiency virus, a papilloma virus, a parainfluenza virus, an influenza virus, a hepatitis virus, a Coxsackie Virus, a herpes virus, a herpes simplex virus, a herpes zoster virus, an Epstein-Barr virus, a varicella virus, a measles virus, a mumps virus, a rubella, rabies virus, a hantavirus, a polio virus, a parvovirus, a polyomavirus, a reovirus, an astrovirus, a filovirus, a picornavirus, or an arenavirus. The bacteria be, but is not limited to, a gram-positive bacteria, a gram-negative bacteria, a drug-resistant bacteria, Escherichia coli (E. coli), Pseudomonas aeruginosa (P. aeruginosa), Staphylococcus aureus (S. aureus), a Group A streptococcus, Group B streptococcus, a S. pneumoniae, Mycobacterium tuberculosis, a Campylobacter jejuni, a Salmonella bacteria, or a Shigella bacteria. The yeast can be, but is not limited to, Candida albicans. The mold can be, but is not limited to, Aspergillus niger.

In some embodiments, the pathogen is a virus, e.g., a DNA or RNA virus. In some embodiments, the virus is an RNA virus, e.g., a single or double-stranded virus. In some embodiments, the RNA virus is a positive-sense single-stranded RNA virus or a negative-sense single-stranded RNA virus. In some embodiments, the virus belongs to the Nidovirales order. In some embodiments, the virus belongs to the Coronaviridae family. In some embodiments, the virus belongs to the alphacoronavirus, betacoronavirus, gammacoronavirus or deltacoronavirus genus. In some embodiments, the alphacoronavirus is, without limitation, human coronavirus 229E, human coronavirus NL63 or transmissible gastroenteritis virus (TGEV). In some embodiments, the betacoronavirus is, without limitation, Severe Acute Respiratory Syndrome Coronavirus (SARS-COV), SARS-CoV-2 (COVID-19), Middle Eastern Respiratory Syndrome Coronavirus (MERS-COV), human coronavirus HKUI, or human coronavirus OC43. In some embodiments, the gammacoronavirus is infectious bronchitis virus (IBV).

In some embodiments, the therapeutic rAAV is designed to express a transgene that is a tumor antigen and is administered to a subject to treat cancer. Expression of the tumor antigen can result the subject mounting an immune response to the tumor. The immune response can be a humoral or cellular immune response. The use of the tolerance-inducing gene therapy vector to induce a tolerance to the therapeutic rAAV vector allows repeat administration of the therapeutic rAAV vector. Repeat administration of the therapeutic rAAV enables repeat administration to delivery the same or different tumor antigens.

In some embodiments, the therapeutic rAAV is designed to express a therapeutic protein or mRNA for treating of a disease or condition or to treat one or more symptoms associated with the disease or condition. In some embodiments, the therapeutic rAAV is configured to express the therapeutic nucleic acid in muscle and/or neuronal tissue. Expression of the therapeutic nucleic acid can result amelioration of a disease or condition or one or more symptoms associated with the disease or condition. Expression of the transgene can provide for a functional protein otherwise missing from the subject. The use of the tolerance-inducing gene therapy vector to induce a tolerance to the therapeutic rAAV vector allows repeat administration of the therapeutic rAAV vector. Repeat administration of the therapeutic rAAV enables repeat dosing to continue to provide the therapeutic nucleic acid.

### VIII. Compositions and Kits

Any of the described therapeutic rAAVs, tolerance-inducing gene therapy vectors, or combinations thereof can be provided in a composition or kit. The compositions can contain one or more carriers or excipients and/or one or more additional therapeutic compounds. The compositions or kits can be used to modulate an immune response in a subject or provide a therapeutic nucleic acid to a subject. The compositions or kits can be used to (a) provide a therapeutic nucleic acid to the subject, (b) induce immune tolerance to a therapeutic rAAV in the subject, (c) induce an immune response, such as a humoral response, to an antigen in the subject, or (e) combinations thereof. Inducing an immune response to an antigen in the subject can be used to vaccinate the subject (e.g., immunize the subject against infection). Providing a therapeutic gene to the subject can be used to treat a disease or condition in the subject.

The therapeutic rAAV and the tolerance-inducing gene therapy vector can be manufacture together or separately. The therapeutic rAAV and the tolerance-inducing lentiviral vector can be manufacture together or separately. Methods for production of multiple rAAV vectors simultaneously are provided in PCT Patent Application No. PCT/US2015/036841, the entire contents of which is incorporated by reference herein. If provided separately, the therapeutic rAAV vector and the tolerance-inducing gene therapy vector can be formulated for co-administration or separate (e.g., sequential) administration.

In some embodiments, the compositions or kits are used to induce a humoral immune response against a virus or other pathogen in a subject. In some embodiments, the compositions or kits are used to induce a humoral immune response against a tumor in a subject. In some embodiments, the compositions or kits are used to provide gene therapy to a subject.

In some embodiments, compositions or kits are provided for modulating an immune response in a subject to immunize the subject against a viral infection. The composition or kit comprises: one or more therapeutic rAAVs comprising a first heterologous nucleic acid region and a second heterologous nucleic acid region, wherein the first heterologous nucleic acid region encodes at least a portion of a viral antigen, but does not encode a functional virus, and wherein the second heterologous nucleic acid region encodes a promoter to drive expression of the at least a portion of the viral antigen; and a tolerance-inducing gene-therapy vector comprising a first heterologous nucleic acid region and a second heterologous nucleic acid region, wherein the first heterologous nucleic acid region encodes at least a portion of a capsid protein of the therapeutic rAAV, wherein the at least a portion of the capsid protein is immunogenic, and wherein the second heterologous nucleic acid region encodes a promoter. In some embodiments, the promoter is a liver-specific or HSC-specific promoter.

In some embodiments, compositions or kits are used to vaccinate a subject against infection by a virus. The virus can be, but is not limited to, coronavirus. The coronavirus can be, but is not limited to SARS-Cov-2. For vaccination against SARS-Cov-2, the therapeutic rAAV encodes an immunogenic polypeptide of SARS-Cov-2. The immunogenic polypeptide of SARS-Cov-2 can be, but is not limited to, at least a portion of a SARS-Cov-2 spike glycoprotein. The at least a portion of the SARS-Cov-2 spike glycoprotein can be, but is not limited to, an S1 subunit of the SARS-Cov-2 spike glycoprotein, or an N-terminal or C-terminal domain of an SI subunit of the SARS-Cov-2 spike glycoprotein.

In some embodiments, compositions or kits for modulating an immune response in a subject to allow repeat administration of a therapeutic rAAV. The compositions or kits comprising a therapeutic rAAV comprising a first heterologous nucleic acid region and a second heterologous nucleic acid region, wherein the first heterologous nucleic acid region encodes all or a portion of a therapeutic nucleic acid, and wherein the second heterologous nucleic acid region encodes a promoter to drive expression of the all or a portion of the therapeutic nucleic acid; and a tolerance-inducing lentiviral vector comprising a first heterologous nucleic acid region and a second heterologous nucleic acid region, wherein the first heterologous nucleic acid region encodes at least a portion of a capsid protein of the therapeutic rAAV, wherein the at least a portion of the capsid protein is immunogenic, and wherein the second heterologous nucleic acid region encodes a promoter that is configured to yield liver-specific or HSC-specific expression of the at least a portion of the capsid protein. The therapeutic nucleic acid can encode a therapeutic RNA or a therapeutic polypeptide. A therapeutic RNA can be, but is not limited to, a mRNA, an aptamer, a microRNA, an siRNA, an RNA interference polynucleotide, an antisense RNA, a ribozyme, or an RNA from one or more parts of a CRISPR/Cas complex (e.g., Cas9, CasX, etc.).

In some embodiments, the compositions or kits comprise a therapeutic rAAV comprising a therapeutic nucleic acid encoding a viral antigen comprising at least a portion of a SARS-Cov-2 polypeptide. In some embodiments, the SARS-Cov-2 polypeptide comprises an S1 subunit of the SARS-Cov-2 spike glycoprotein. In some embodiments, the SARS-Cov-2 polypeptide comprises an N-terminal or C-terminal domain of an S1 subunit of the SARS-Cov-2 spike glycoprotein. In some embodiments, the kits further comprise instructions for use in treating COVID-19.

In some embodiments, a composition or kit for vaccinating a subject against SARS-CoV-2 comprises: a first (therapeutic) rAAV vector of a first serotype encoding a SARS-COV-2 soluble spike protein, the expression of which is driven, for example, by a CMV promoter and a tolerance-inducing lentiviral vector of a second serotype different than the serotype or the therapeutic rAAV encoding all or an immunogenic portion of an rAAV capsid protein of the first serotype, the expression of which is driven, for example by a liver-specific promoter. Administration of the tolerance-inducing lentiviral vector results in induction of specific immune tolerance to the capsid protein of the therapeutic rAAV, thereby reducing or eliminating immune response to the therapeutic rAAV vector.

In some embodiments, one or more pharmaceutically acceptable excipients (including vehicles, carriers, diluents, and/or delivery polymers) are added to the pharmaceutical compositions including a therapeutic and/or tolerance inducing rAAVs, thereby forming a pharmaceutical formulation suitable for in vivo delivery to a subject, such as a human.

A pharmaceutical composition or medicament includes a pharmacologically effective amount of at least one of the therapeutic and/or tolerance inducing rAAV and optionally one or more pharmaceutically acceptable excipients. Pharmaceutically acceptable excipients (excipients) are substances other than the Active Pharmaceutical ingredient (API, therapeutic product) that are intentionally included in the drug delivery system. Excipients do not exert or are not intended to exert a therapeutic effect at the intended dosage. Excipients may act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

The pharmaceutical compositions can contain other additional components commonly found in pharmaceutical compositions. Such additional components can include, but are not limited to: anti-pruritics, astringents, local anesthetics, or anti-inflammatory agents (e.g., antihistamine, diphenhydramine, etc.).

The carrier can be, but is not limited to, a solvent or dispersion medium containing, for example, water, ethanol, polyol (for example, glycerol, propylene glycol, and liquid polyethylene glycol), and suitable mixtures thereof. A carrier may also contain adjuvants such as preservatives, wetting agents, emulsifying agents, and dispersing agents. A carrier may also contain isotonic agents, such as sugars, polyalcohols, sodium chloride, and the like into the compositions.

Pharmaceutically acceptable refers to those properties and/or substances which are acceptable to the subject from a pharmacological/toxicological point of view. The phrase pharmaceutically acceptable refers to molecular entities, compositions, and properties that are physiologically tolerable and do not typically produce an allergic or other untoward or toxic reaction when administered to a subject. In some embodiments, a pharmaceutically acceptable compound is approved by a regulatory agency of the Federal or a state government or listed in the U.S. Pharmacopeia or other generally recognized pharmacopeia for use in animals and more particularly in humans.

In some embodiments, the kits comprise one or more containers or receptacles comprising one or more doses of any of the described therapeutic and/or tolerance inducing rAAVs. Such kits may be therapeutic in nature. In certain embodiments, the kit contains a unit dosage, meaning a predetermined amount of a composition comprising, for example, a described therapeutic and/or tolerance inducing rAAV with or without one or more additional agents.

One or more of the components of a kit can be provided in one or more liquid or frozen solvents. The solvent can be aqueous or non-aqueous. The formulation in the kit can also be provided as dried powder(s) or in lyophilized form that can be reconstituted upon addition of an appropriate solvent.

In some embodiments, a kit comprises a label, marker, package insert, bar code and/or reader indicating directions of suitable usage of the kit contents. In some embodiments, the kit may comprise a label, marker, package insert, bar code and/or reader indicating that the kit contents may be administered in accordance with a certain dosage or dosing regimen to treat a subject.

### IX. Administration

The first (therapeutic) rAAV and the tolerance-inducing gene therapy vector can be co-administered or sequentially administered. For co-administration, the therapeutic and tolerance-inducing gene therapy vector can be combined prior to administration. In some embodiments, a tolerance inducing gene therapy vector can be combined with two or more therapeutic rAAVs, wherein the two or more rAAVs encoded different therapeutic nucleic acids.

The tolerance-inducing gene therapy vectors and therapeutic rAAVs can be administered by a variety of routes. Administration routes included, but are not limited to, intravenous, intra-arterial, subcutaneous, intramuscular, intrahepatic, intraperitoneal and/or local delivery to a target tissue. In some embodiments, a plurality of injections, or other administration types, are provided, for example 2, 3, 4, 5, 6, 7, 8, 9, 10 or more injections. Depending on the embodiment, the therapeutic and tolerance-inducing gene therapy vector need not be administered the same number of times (e.g., the tolerance-inducing gene therapy vector may be administered 1 time, and the therapeutic rAAV may be administered two or more times). In some embodiments, the dosing is intramuscular administration.

In some embodiments, the tolerance-inducing gene therapy vectors administered by systemic injection. In some embodiments, the tolerance inducing gene therapy vectors administered by intravenous injection.

The dose of a therapeutic rAAV and/or the tolerance-inducing rAAV can be between about 1×10¹⁰ vector genomes (VG) and about 1×10¹⁴ VG, including about 5×10¹⁰ VG, 1×10¹¹, 5×10¹¹, 1×10¹², 5×10¹², 1×10¹³, 5×10¹³, 1×10¹⁴ and any dose in between or inclusive of the doses listed. The dose of the therapeutic rAAV and the dose of the tolerance inducing rAAV can base on relation to one another, or based on the target tissue for expression. For example the ratio of the therapeutic rAAV to the tolerance inducing rAAV can be about 1:1, 1:2, 1:4, 1:5, 1:7, 1:10, 1:20, 1:50, 1:100, 1:200, 1:500, 500:1, 200:1, 100:1, 50:1, 20:1, 10:1, 7:1, 5:1, 4:1, 2:1, or any ratio in between or inclusive of those ratios listed. In some embodiments, depending on the subject to be treated, the dose is on a vector genome/kilogram (VG/kg) body mass basis.

The therapeutic rAAV may be administered on the same day as the tolerance-including gene therapy vector, prior to administration of the tolerance-inducing gene therapy vector, or after administration of the tolerance-inducing gene therapy vector. The tolerance-inducing gene therapy vector may be administered on the same day as the therapeutic rAAV, prior to administration of the therapeutic rAAV, or after administration of the therapeutic rAAV. The tolerance-including gene therapy vector may be administered 1 day, 2 days, 3 days, 4 days, 5 days, 6 days, 7 days, 8 days, 9 days, 10 days, 11 days, 12 days, 13 days, 14 days, 1 week, 2 weeks, 3 weeks, 4 weeks, 5 weeks, 6 weeks, 7 weeks, 8 weeks, 1 month 2 months, 3 months, 4 months, 5 months, 6 months or more prior to administration of the therapeutic rAAV. The tolerance-including gene therapy vector may be administered 1 day, 2 days, 3 days, 4 days, 5 days, 6 days, 7 days, 8 days, 9 days, 10 days, 11 days, 12 days, 13 days, 14 days, 1 week, 2 weeks, 3 weeks, 4 weeks, 5 weeks, 6 weeks, 7 weeks, 8 weeks, 1 month 2 months, 3 months, 4 months, 5 months, 6 months or more after administration of the therapeutic rAAV.

The subject treated by the present methods can be any suitable subject in need of treatment with the therapeutic nucleic acid delivered by a therapeutic rAAV. In some embodiments, the subject is a mammal. In some embodiments, the subject is a human subject. In some embodiments, the subject treated by the present methods can be any suitable subject in need of treatment or prevention of viral infection or other pathogen infection. In some embodiments, the subject may have already been exposed to a given virus, or variant thereof. In some embodiments, the subject has one or more comorbidities, such as, but not limited to a cardiorespiratory dysfunction, hypertension, diabetes, and/or coronary heart disease. “Subject” and “patient” are used interchangeably herein.

In some embodiments, the tolerance-including gene therapy vectors and therapeutic rAAV particles described herein are administered to otherwise healthy individuals in order to induce development of a humoral immune response against a transgene encoded by the rAAV vector, such as an pathogen antigen.

In certain embodiments, treatment of a subject as described herein achieves one, two, three, four, or more of the following effects, including, for example: (i) reduction or amelioration the severity of disease or symptom associated therewith; (ii) reduction in the duration of a symptom associated with a disease; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease; (xi) an enhancement, improvement, supplementation, complementation, or augmentation of the prophylactic or therapeutic effect(s) of another therapy.

### X. Combination Therapies

The tolerance-including gene therapy vectors and therapeutic rAAVs as disclosed can be administered in combination with one or more additional therapeutic agents.

In some embodiments, the additional therapeutic agent comprises an anti-inflammatory agent. The anti-inflammatory agent can be, but is not limited to, a corticosteroid, cortisone hydrocortisone, hydrocortisone-21-monoesters (e.g., hydrocortisone-21-acetate, hydrocortisone-21-butyrate, hydrocortisone-21-propionate, hydrocortisone-21-valerate, etc.), hydrocortisone-17,21-diesters (e.g., hydrocortisone-17,21-diacetate, hydrocortisone-17-acetate-21-butyrate, hydrocortisone-17,21-dibutyrate, etc.), alclometasone, dexamethasone, flumethasone, prednisolone, methylprednisolone, betamethasone, typically as betamethasone benzoate or betamethasone diproprionate; fluocinonide; prednisone; and triamcinolone, typically as triamcinolone acetonide. In some embodiments, the anti-inflammatory agent is a mast cell degranulation inhibitor, such as, without limitation, cromolyn (5,5′-(2-hydroxypropane-1,3-diyl)bis(oxy)bis(4-oxo-4H-chromene-2-carboxylic acid) (also known as cromoglycate), and 2-carboxylatochromon-5′-yl-2-hydroxypropane derivatives such as bis(acetoxymethyl), disodium cromoglycate, nedocromil (9-ethyl-4,6-dioxo-10-propyl-6,9-dihydro-4H-pyrano[3,2-g]quinoline-2,8-dicarboxylic acid) and tranilast (2-{[(2E)-3-(3,4-dimethoxyphenyl) prop-2-enoyl]amino}), and lodoxamide (2-[2-chloro-5-cyano-3-(oxaloamino)anilino]-2-oxoacetic acid). In some embodiments, the anti-inflammatory agent is a nonsteroidal anti-inflammatory drugs (NSAIDs), such as, without limitation, aspirin compounds (acetylsalicylates), non-aspirin salicylates, diclofenac, diflunisal, etodolac, fenoprofen, flurbiprofen, ibuprofen, indomethacin, ketoprofen, meclofenamate, naproxen, naproxen sodium, phenylbutazone, sulindac, and tometin.

In some embodiments, the anti-inflammatory agent comprises an antihistamine. The antihistamine can be, but is not limited to, clemastine, clemastine fumarate (2 (R)-[2-[1-(4-Chlorophenyl)-1-phenyl-ethoxy]ethyl-1-methylpyrrolidine), dexmedetomidine, doxylamine, loratidine, desloratidine and promethazine, and diphenhydramine, or pharmaceutically acceptable salts, solvates or esters thereof. In some embodiments, the antihistamine includes, without limitation, azatadine, azelastine, burfroline, cetirizine, cyproheptadine, doxantrozole, etodroxizine, forskolin, hydroxyzine, ketotifen, oxatomide, pizotifen, proxicromil, N,N′-substituted piperazines or terfenadine. In some embodiments, the antihistamine is an H1 antagonist, such as, but not limited to, cetirizine, chlorpheniramine, dimenhydrinate, diphenhydramine, fexofenadine, hydroxyzine, orphenadrine, pheniramine, and doxylamine. In some embodiments, the antihistamine is an H2 antagonist, such as, but not limited to, cimetidine, famotidine, lafutidine, nizatidine, ranitidine, and roxatidine.

In some embodiments, the additional therapeutic agent comprises an antiviral agent, including antiretroviral agents. Suitable antiviral agents include, without limitation, remdesivir, acyclovir, famcyclovir, ganciclovir, foscarnet, idoxuridine, sorivudine, trifluorothymidine, valacyclovir, vidarabine, didanosine, dideoxyinosine, stavudine, zalcitabine, zidovudine, amantadine, interferon alpha, ribavirin and rimantadine.

In some embodiments, the additional therapeutic agent comprises an antibiotic. Non-limiting examples of suitable antibiotics include beta-lactams such as penicillins, aminopenicillins (e.g., amoxicillin, ampicillin, hetacillin, etc.), penicillinase resistant antibiotics (e.g., cloxacillin, dicloxacillin, methicillin, nafcillin, oxacillin, etc.), extended spectrum antibiotics (e.g., axlocillin, carbenicillin, mezlocillin, piperacillin, ticarcillin, etc.); cephalosporins (e.g., cefadroxil, cefazolin, cephalixin, cephalothin, cephapirin, cephradine, cefaclor, cefacmandole, cefmetazole, cefonicid, ceforanide, cefotetan, cefoxitin, cefprozil, cefuroxime, loracarbef, cefixime, cefoperazone, cefotaxime, cefpodoxime, ceftazidime, ceftiofur, ceftizoxime, ceftriaxone, moxalactam, etc.); monobactams such as aztreonam; Carbapenems such as imipenem and eropenem; quinolones (e.g., ciprofloxacin, enrofloxacin, difloxacin, orbifloxacin, marbofloxacin, etc.); chloramphenicols (e.g., chloramphenicol, thiamphenicol, florfenicol, etc.); tetracyclines (e.g., chlortetracycline, tetracycline, oxytetracycline, doxycycline, minocycline, etc.); macrolides (e.g., erythromycin, tylosin, tlimicosin, clarithromycin, azithromycin, etc.); lincosamides (e.g., lincomycin, clindamycin, etc.); aminoglycosides (e.g., gentamicin, amikacin, kanamycin, apramycin, tobramycin, neomycin, dihydrostreptomycin, paromomycin, etc.); sulfonamides (e.g., sulfadmethoxine, sfulfamethazine, sulfaquinoxaline, sulfamerazine, sulfathiazole, sulfasalazine, sulfadiazine, sulfabromomethazine, suflaethoxypyridazine, etc.); glycopeptides (e.g., vancomycin, teicoplanin, ramoplanin, and decaplanin; and other antibiotics (e.g., rifampin, nitrofuran, virginiamycin, polymyxins, tobramycin, etc.).

In some embodiments, the additional therapeutic agent comprises an antifungal agent, such as, but not limited to, itraconazole, ketoconazole, fluoconazole, and amphotericin B. In some embodiments, the therapeutic agent is an antiparasitic agents, such as, but not limited to, the broad spectrum antiparasitic medicament nitazoxanide; antimalarial drugs and other antiprotozoal agents (e.g., artemisins, mefloquine, lumefantrine, tinidazole, and miltefosine); anthelminthics such as mebendazole, thiabendazole, and ivermectin; and antiamoebic agents such as rifampin and amphotericin B.

In some embodiments, the additional therapeutic agent comprises an analgesic agent, including, without limitation, opioid analgesics such as alfentanil, buprenorphine, butorphanol, codeine, drocode, fentanyl, hydrocodone, hydromorphone, levorphanol, meperidine, methadone, morphine, nalbuphine, oxycodone, oxymorphone, pentazocine, propoxyphene, sufentanil, and tramadol; and nonopioid analgesics such as apazone, etodolac, diphenpyramide, indomethacin, meclofenamate, mefenamic acid, oxaprozin, phenylbutazone, piroxicam, and tolmetin.

### XI. Sequences

In some embodiments, there are provided amino acid sequences that correspond to any of the nucleic acids disclosed herein (and/or included in the accompanying sequence listing), while accounting for degeneracy of the nucleic acid code. Furthermore, those sequences (whether nucleic acid or amino acid) that vary from those expressly disclosed herein (and/or included in the accompanying sequence listing), but have functional similarity or equivalency are also contemplated within the scope of the present disclosure. The foregoing includes mutants, truncations, substitutions, or other types of modifications.

In accordance with some embodiments described herein, any of the sequences may be used, or a truncated or mutated form of any of the sequences disclosed herein (and/or included in the accompanying sequence listing) may be used and in any combination.

Any titles or subheadings used herein are for organization purposes and should not be used to limit the scope of embodiments disclosed herein.

### EXAMPLES

### Example 1

Assessment of safety of vaccination of an individual with a rAAV2-LSP (GAA)-AAV1-VP3 vector in order to induce immune tolerance to subsequent intramuscular administration of a recombinant adeno-associated virus vector, rAAV1-CMV-SARS-COV-2, in healthy adult volunteers.

Two dose levels (1.0×10¹³ and 5.0×10¹² VG) will be studied. One goal is to determine the degree of immune tolerance induced to rAAV1 vectors, through the liver-specific expression of an immunogenic portion of AAV1 VP3 capsid protein.

The study population will be made up of 6 subjects, male or female, 18-75 years of age, three with an anti-AAV1 titer of <100U/ml and three with an anti-AAV1 titer of 100-500 U/ml. All subjects will be healthy volunteers who test negative for SARS-COV-2 at screening. An intravenous injection of the requisite dose will be administered to three subjects at each dose level. One week later, each subject will be administered a corresponding dose (low to low and high to high) rAAV1-CMV-SARS-COV-2 vector delivered intramuscularly (either 1.0×10¹³ and 5.0×10¹² VG). One goal is to determine the safety of in healthy adults as well as to determine the efficacy of rAAV1-CMV-SARS-COV-2 vector in producing the anti-SARS-COV-2 antibody.

Following screening visit and vaccine administration at day 0, subjects will be evaluated at a clinical site on days 3, 7, 14, 28 and 90, of the trial. Following day 90, subjects will be evaluated by the study nurse by telephone, or video conference when available. Subjects may be provided with a sample collection kit if they are experiencing any long-term side-effects. Blood samples will be collected at an outpatient facility and shipped to the clinical site for analysis. At each visit to the clinical site, subjects will have a physical examination and laboratory evaluation of chemistry and hematology.

In addition, at days 3, 7, 14, 28 and 90 the presence of vector in peripheral blood will be evaluated. Testing for the presence of anti-AAV1 and anti-SARS-COV-2 antibodies will occur at screening, day 0 (pre-injection), and days 3, 7, 14, 28 and 90.

Safety will be assessed by measurement of changes in serum chemistries, coagulation and hematology, urinalysis, and immunologic response to SARS-COV-2 and AAV1 as well as reported subject history of any symptoms.

Impact of the vaccine in promoting the production of anti-SARS-COV-2 antibodies will be assessed by measurement of IgG and IgM for SARS-COV-2 in the blood. Testing will be done at screening, baseline and 3, 7, 14, 28, and 90 days following vaccine administration.

### Example 2

A clinical study will be performed to verify the safety and efficacy of certain embodiments disclosed herein. The study will be conducted in healthy control adult subjects at two dosing levels, 5×10¹² VG and 1×10¹³ VG delivered intramuscularly in the quadriceps muscle. This study will test several important concepts leading to immediate immunization of susceptible individuals. The critical advantage of an AAV-based vaccine is that single administration in a 1.0 ml IM injection will lead to ongoing expression of the modified soluble coronavirus spike protein (or other viral protein) and elicit a potent anti-spike polyclonal response. Based on non-clinical studies, the level of antibody should exceed the threshold for effective neutralization of SARS-COV-2.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. In addition, where features or aspects of the disclosure are described in terms of Markush groups, those skilled in the art will recognize that the disclosure is also thereby described in terms of any individual member or subgroup of members of the Markush group.

### Example 3

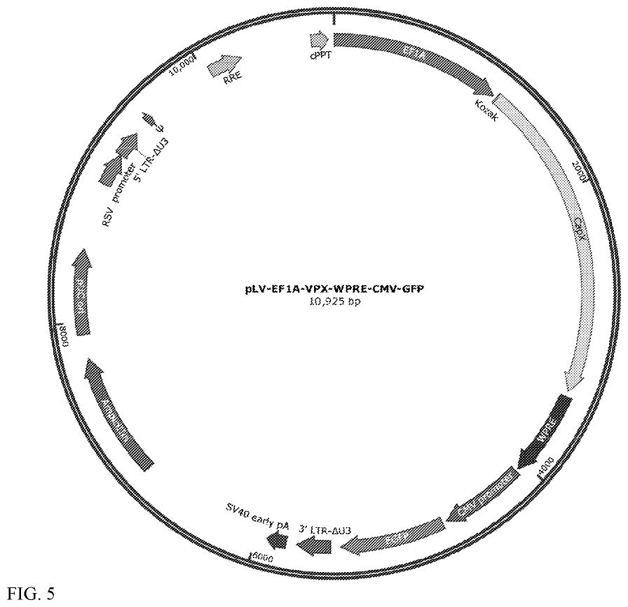

Construct design. Expression constructs which code for AAV viral capsids were codon optimized for expression in human tissues and were subcloned into a plasmid backbone suitable for production of AAV. The constructs were engineered to comprise the elements as provided in Tables 1-6 below. Schematic representations of the constructs is provided in FIGS. 5-11. Similar constructs are readily made for production of lentiviral vectors and/or non-viral vectors using the AAV capsid-encoding sequences, and optionally the promoter and/or 3′UTR/poly A sequences. Additional sequences know in the art an appropriate to the delivery vector are readily incorporated. In some embodiments, the expression construct comprises a promoter sequence and capsid (VP1 or VP3) sequence of any of the constructions shown in Table 1-6 and FIGS. 5-11.

AAV production. Recombinant AAV (rAAV) particles comprising each of the constructs are made by suspension transfection of Expi293T cells with the pTR2-LSP-VP1 (AAV9/rh74) constructs and other plasmids needed for rAAV production (e.g., comprising rep and cap expression cassettes) to generate three groups of rAAV expressing (1) AAV9 capsid proteins; (2) rh74 capsid proteins; and (3) rh74 variant capsid proteins. Vector is isolated using a capture column followed by an anion exchange column and purified using a cesium chloride gradient to a titer of 2-5×10¹³ viral genomes/mL.

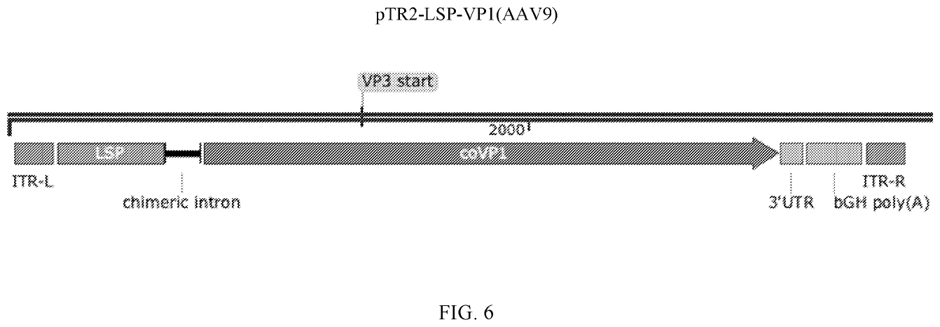

TABLE 1
Construct 1 (pTR2-LSP-VP1 (AAV9); FIG. 6)

| SEQ | | |
|---|---|---|
| ID | Elements | |
| NO: | (5′→3′) | Sequence |
| 13 | ITR-L | TTGGCCACTCCCTCTCTGCGCGCTCGCTCGCTCACTGAGGCCGGGCGACCAAAG |
| | | GTCGCCCGACGCCCGGGCTTTGCCCGGGCGGCCTCAGTGAGCGAGCGAGCGCG |
| | | CAGAGAGGGAGTGGCCAACTCCATCACTAGGGGTTCCT |
| 3 | LSP | CCCTAAAATGGGCAAACATTGCAAGCAGCAAACAGCAAACACACAGCCCTCCC |
| | promoter | TGCCTGCTGACCTTGGAGCTGGGGCAGAGGTCAGAGACCTCTCTGGGGACTGTC |
| | | CCAGGTCAGTGGTGGTGCCTGAAGCTGAGGAGACAGGGCCCTGTCCTCGTCCGT |
| | | ATTTAAGCAGTGGATCCAGAGGGGCAACGGGGGAGGCTGCTGGTGAATATTAA |
| | | CCAAGGTCACCCCAGTTATCGGAGGAGCAAACAGGGGCTAAGTCCACTGGCTG |
| | | GGATCTGAGTCGCCCGCCTACGCTGCCCGGACGCTTTGCCTGGGCAGTGTACAG |
| | | CTTCCACTGCACTTACCGAAAGGAGTCATTGTAGGGCCCTGTCTCCTCAGCTTCA |
| | | GGCACCACCACTGACCTGGGACAGTGAATCCGGA |
| 14 | sd/sa | GTAAGTATCAAGGTTACAAGACAGGTTTAAGGAGACCAATAGAAACTGGGCTT |
| | | GTCGAGACAGAGAAGACTCTTGCGTTTCTGATAGGCACCTATTGGTCTTACTGA |
| | | CATCCACTTTGCCTTTCTCTCCACAG |
| 15 | AAV9 | ATGGCCGCCGACGGCTACCTGCCTGACTGGCTGGAAGATAATCTGAGCGAGGG |
| | coVP1 | CATCCGGGAATGGTGGGCCCTGAAGCCCGGCGCTCCTCAACCTAAGGCAAACC |
| | | AGCAGCACCAGGACAACGCCAGAGGCCTCGTGCTGCCTGGATATAAGTACCTG |
| | | GGACCTGGCAACGGCCTGGACAAGGGCGAGCCCGTGAACGCCGCTGATGCCGC |
| | | TGCTCTGGAGCACGACAAGGCCTACGACCAGCAGCTGAAAGCCGGCGATAACC |
| | | CCTACCTGAAGTACAACCACGCCGACGCCGAGTTCCAAGAGAGACTGAAGGAA |
| | | GATACCAGCTTTGGCGGAAATCTGGGCAGAGCCGTGTTCCAGGCCAAGAAGCG |
| | | CCTGCTGGAACCTCTGGGACTGGTCGAGGAAGCCGCCAAGACAGCCCCTGGCA |
| | | AGAAAAGACCTGTGGAACAGAGCCCTCAGGAGCCCGACAGCTCCGCCGGAATC |
| | | GGCAAAAGCGGCGCCCAGCCCGCCAAAAAGCGGCTGAACTTCGGCCAGACCGG |
| | | CGACACAGAGTCTGTGCCAGATCCTCAGCCTATCGGCGAGCCACCTGCTGCCCC |
| | | CAGCGGTGTTGGCAGCCTGACCATGGCCTCTGGCGGCGGCGCCCCAGTGGCCGA |
| | | CAACAACGAGGGAGCTGACGGCGTGGGCTCTTCCAGCGGCAATTGGCACTGCG |
| | | ACAGCCAATGGCTGGGAGATAGAGTGATCACCACCAGCACAAGAACCTGGGCT |
| | | CTGCCTACATACAACAACCACCTGTACAAACAGATCAGCAACTCCACTAGCGGT |
| | | GGCAGCAGCAACGACAATGCCTACTTCGGCTACAGCACCCCTTGGGGATACTTC |
| | | GACTTTAACAGATTCCACTGTCACTTCTCTCCTAGAGATTGGCAGAGACTGATC |
| | | AACAACAACTGGGGCTTCCGGCCTAAGCGGCTTAACTTCAAGCTGTTCAACATC |
| | | CAGGTCAAAGAGGTGACAGATAACAATGGAGTGAAGACCATCGCCAACAACCT |
| | | GACAAGCACTGTGCAGGTGTTCACCGACAGTGATTACCAGCTGCCATACGTGCT |
| | | GGGCTCTGCCCACGAGGGCTGCCTGCCTCCTTTCCCTGCTGACGTGTTCATGATC |
| | | CCTCAGTATGGCTACCTGACCCTGAATGATGGCTCCCAGGCCGTGGGCAGAAGC |
| | | TCCTTCTACTGCCTGGAATACTTTCCTAGCCAAATGCTGAGAACAGGGAACAAC |
| | | TTCCAGTTTAGCTACGAGTTCGAGAACGTGCCCTTCCACAGCAGCTACGCCCAT |
| | | TCTCAAAGCCTGGACAGACTGATGAATCCCCTGATCGACCAGTACCTGTACTAC |
| | | CTCAGCAAGACCATCAACGGCAGTGGCCAGAACCAGCAGACCCTGAAGTTCAG |
| | | CGTGGCGGGCCCTTCTAATATGGCAGTGCAGGGCCGCAACTACATCCCAGGCCC |
| | | CTCCTATAGACAGCAGCGGGTGTCTACAACCGTGACCCAGAACAACAACTCAG |
| | | AGTTTGCCTGGCCTGGCGCCTCTAGCTGGGCCCTGAACGGCAGAAATAGCCTGA |
| | | TGAACCCTGGCCCCGCTATGGCCAGCCACAAGGAAGGCGAGGACCGGTTCTTCC |
| | | CCCTGAGCGGCTCTCTGATTTTCGGCAAGCAGGGCACCGGCAGGGACAACGTGG |
| | | ACGCCGATAAGGTGATGATCACCAACGAGGAAGAAATCAAGACAACCAATCCT |
| | | GTGGCCACCGAGAGCTACGGCCAGGTCGCCACAAATCACCAGAGCGCCCAGGC |
| | | TCAGGCCCAAACAGGCTGGGTTCAGAATCAGGGCATCCTGCCCGGCATGGTGTG |
| | | GCAGGACAGAGATGTGTACCTGCAAGGCCCTATCTGGGCCAAGATCCCCCACAC |
| | | CGACGGAAACTTCCACCCCAGCCCTCTCATGGGCGGCTTTGGCATGAAGCACCC |
| | | TCCACCACAGATCCTGATCAAGAACACACCTGTGCCAGCCGATCCTCCTACCGC |
| | | CTTTAACAAGGACAAGCTGAACAGCTTCATCACACAGTACAGCACCGGACAAG |
| | | TGTCCGTGGAAATCGAGTGGGAGCTGCAGAAGGAAAACAGCAAAAGATGGAAC |
| | | CCGGAAATTCAGTACACCAGCAACTACTACAAAAGCAACAACGTGGAATTCGC |
| | | CGTGAACACCGAGGGAGTGTATTCTGAGCCTCGGCCCATCGGAACCAGATACCT |
| | | GACCCGGAACCTGTGA |
| 16 | AAV9 | MAADGYLPDWLEDNLSEGIREWWALKPGAPQPKANQQHQDNARGLVLPGYKYL |
| | coVP1 | GPGNGLDKGEPVNAADAAALEHDKAYDQQLKAGDNPYLKYNHADAEFQERLKE |
| | | DTSFGGNLGRAVFQAKKRLLEPLGLVEEAAKTAPGKKRPVEQSPQEPDSSAGIGKS |
| | | GAQPAKKRLNFGQTGDTESVPDPQPIGEPPAAPSGVGSLTMASGGGAPVADNNEGA |
| | | DGVGSSSGNWHCDSQWLGDRVITTSTRTWALPTYNNHLYKQISNSTSGGSSNDNA |
| | | YFGYSTPWGYFDFNRFHCHFSPRDWQRLINNNWGFRPKRLNFKLFNIQVKEVTDN |
| | | NGVKTIANNLTSTVQVFTDSDYQLPYVLGSAHEGCLPPFPADVFMIPQYGYLTLND |
| | | GSQAVGRSSFYCLEYFPSQMLRTGNNFQFSYEFENVPFHSSYAHSQSLDRLMNPLID |
| | | QYLYYLSKTINGSGQNQQTLKFSVAGPSNMAVQGRNYIPGPSYRQQRVSTTVTQNN |
| | | NSEFAWPGASSWALNGRNSLMNPGPAMASHKEGEDRFFPLSGSLIFGKQGTGRDN |
| | | VDADKVMITNEEEIKTTNPVATESYGQVATNHQSAQAQAQTGWVQNQGILPGMV |
| | | WQDRDVYLQGPIWAKIPHTDGNFHPSPLMGGFGMKHPPPQILIKNTPVPADPPTAF |
| | | NKDKLNSFITQYSTGQVSVEIEWELQKENSKRWNPEIQYTSNYYKSNNVEFAVNTE |
| | | GVYSEPRPIGTRYLTRNL |
| 17 | 3′ UTR | TTGCTTGTTAATCAATAAACCGTTTAATTCGTTTCAGTTGAACTTTGGTCTCTGC |
| | | GTATTTCTTTCTTATCTAGTTTCCATGCTCTAG |
| 18 | polyA | CTGTGCCTTCTAGTTGCCAGCCATCTGTTGTTTGCCCCTCCCCCGTGCCTTCCTTG |
| | | ACCCTGGAAGGTGCCACTCCCACTGTCCTTTCCTAATAAAATGAGGAAATTGCA |
| | | TCGCATTGTCTGAGTAGGTGTCATTCTATTCTGGGGGGTGGGGTGGGGCAGGAC |
| | | AGCAAGGGGGAGGATTGGGAAGACAATAGCAGGCATGCTGGGGA |
| 19 | ITR-R | AGGAACCCCTAGTGATGGAGTTGGCCACTCCCTCTCTGCGCGCTCGCTCGCTCA |
| | | CTGAGGCCGGGCGACCAAAGGTCGCCCGACGCCCGGGCTTTGCCCGGGCGGCC |
| | | TCAGTGAGCGAGCGAGCGCGCAGAGAGGGAGTGGCCAA |

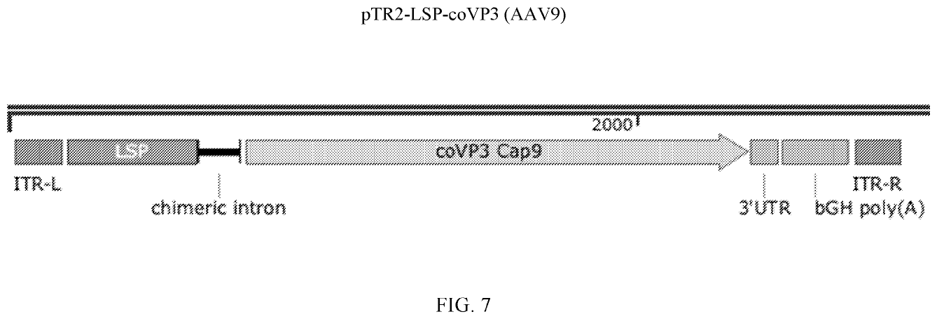

TABLE 2
Construct 2 (pTR2-LSP-coVP3 (AAV9); FIG. 7)

| SEQ | | |
|---|---|---|
| ID | Elements | |
| NO: | (5′→3′) | Sequence |
| 13 | ITR-L | TTGGCCACTCCCTCTCTGCGCGCTCGCTCGCTCACTGAGGCCGGGCGACCAAAG |
| | | GTCGCCCGACGCCCGGGCTTTGCCCGGGCGGCCTCAGTGAGCGAGCGAGCGCG |
| | | CAGAGAGGGAGTGGCCAACTCCATCACTAGGGGTTCCT |
| 3 | LSP | CCCTAAAATGGGCAAACATTGCAAGCAGCAAACAGCAAACACACAGCCCTCCC |
| | promoter | TGCCTGCTGACCTTGGAGCTGGGGCAGAGGTCAGAGACCTCTCTGGGGACTGTC |
| | | CCAGGTCAGTGGTGGTGCCTGAAGCTGAGGAGACAGGGCCCTGTCCTCGTCCGT |
| | | ATTTAAGCAGTGGATCCAGAGGGGCAACGGGGGAGGCTGCTGGTGAATATTAA |
| | | CCAAGGTCACCCCAGTTATCGGAGGAGCAAACAGGGGCTAAGTCCACTGGCTG |
| | | GGATCTGAGTCGCCCGCCTACGCTGCCCGGACGCTTTGCCTGGGCAGTGTACAG |
| | | CTTCCACTGCACTTACCGAAAGGAGTCATTGTAGGGCCCTGTCTCCTCAGCTTCA |
| | | GGCACCACCACTGACCTGGGACAGTGAATCCGGA |
| 14 | sd/sa | GTAAGTATCAAGGTTACAAGACAGGTTTAAGGAGACCAATAGAAACTGGGCTT |
| | | GTCGAGACAGAGAAGACTCTTGCGTTTCTGATAGGCACCTATTGGTCTTACTGA |
| | | CATCCACTTTGCCTTTCTCTCCACAG |
| 20 | AAV9 | ATGGCCAGCGGAGGAGGCGCTCCTGTGGCCGACAACAATGAGGGAGCCGACGG |
| | coVP3 | CGTCGGCAGCTCCAGCGGCAATTGGCACTGCGACAGCCAATGGCTGGGAGATA |
| | | GAGTGATCACCACCAGCACAAGAACCTGGGCTCTGCCTACATACAACAACCACC |
| | | TGTACAAACAGATCAGCAACTCCACTAGCGGTGGCAGCAGCAACGACAATGCC |
| | | TACTTCGGCTACAGCACCCCTTGGGGATACTTCGACTTTAACAGATTCCACTGTC |
| | | ACTTCTCTCCTAGAGATTGGCAGAGACTGATCAACAACAACTGGGGCTTCCGGC |
| | | CTAAGCGGCTTAACTTCAAGCTGTTCAACATCCAGGTCAAAGAGGTGACAGATA |
| | | ACAATGGAGTGAAGACCATCGCCAACAACCTGACAAGCACTGTGCAGGTGTTC |
| | | ACCGACAGTGATTACCAGCTGCCATACGTGCTGGGCTCTGCCCACGAGGGCTGC |
| | | CTGCCTCCTTTCCCTGCTGACGTGTTCATGATCCCTCAGTATGGCTACCTGACCC |
| | | TGAATGATGGCTCCCAGGCCGTGGGCAGAAGCTCCTTCTACTGCCTGGAATACT |
| | | TTCCTAGCCAAATGCTGAGAACAGGGAACAACTTCCAGTTTAGCTACGAGTTCG |
| | | AGAACGTGCCCTTCCACAGCAGCTACGCCCATTCTCAAAGCCTGGACAGACTGA |
| | | TGAATCCCCTGATCGACCAGTACCTGTACTACCTCAGCAAGACCATCAACGGCA |
| | | GTGGCCAGAACCAGCAGACCCTGAAGTTCAGCGTGGCGGGCCCTTCTAATATGG |
| | | CAGTGCAGGGCCGCAACTACATCCCAGGCCCCTCCTATAGACAGCAGCGGGTGT |
| | | CTACAACCGTGACCCAGAACAACAACTCAGAGTTTGCCTGGCCTGGCGCCTCTA |
| | | GCTGGGCCCTGAACGGCAGAAATAGCCTGATGAACCCTGGCCCCGCTATGGCCA |
| | | GCCACAAGGAAGGCGAGGACCGGTTCTTCCCCCTGAGCGGCTCTCTGATTTTCG |
| | | GCAAGCAGGGCACCGGCAGGGACAACGTGGACGCCGATAAGGTGATGATCACC |
| | | AACGAGGAAGAAATCAAGACAACCAATCCTGTGGCCACCGAGAGCTACGGCCA |
| | | GGTCGCCACAAATCACCAGAGCGCCCAGGCTCAGGCCCAAACAGGCTGGGTTC |
| | | AGAATCAGGGCATCCTGCCCGGCATGGTGTGGCAGGACAGAGATGTGTACCTG |
| | | CAAGGCCCTATCTGGGCCAAGATCCCCCACACCGACGGAAACTTCCACCCCAGC |
| | | CCTCTCATGGGCGGCTTTGGCATGAAGCACCCTCCACCACAGATCCTGATCAAG |
| | | AACACACCTGTGCCAGCCGATCCTCCTACCGCCTTTAACAAGGACAAGCTGAAC |
| | | AGCTTCATCACACAGTACAGCACCGGACAAGTGTCCGTGGAAATCGAGTGGGA |
| | | GCTGCAGAAGGAAAACAGCAAAAGATGGAACCCGGAAATTCAGTACACCAGCA |
| | | ACTACTACAAAAGCAACAACGTGGAATTCGCCGTGAACACCGAGGGAGTGTAT |
| | | TCTGAGCCTCGGCCCATCGGAACCAGATACCTGACCCGGAACCTGTGA |
| 21 | AAV9 | MASGGGAPVADNNEGADGVGSSSGNWHCDSQWLGDRVITTSTRTWALPTYNNHL |
| | coVP3 | YKQISNSTSGGSSNDNAYFGYSTPWGYFDFNRFHCHFSPRDWQRLINNNWGFRPKR |
| | | LNFKLFNIQVKEVTDNNGVKTIANNLTSTVQVFTDSDYQLPYVLGSAHEGCLPPFPA |
| | | DVFMIPQYGYLTLNDGSQAVGRSSFYCLEYFPSQMLRTGNNFQFSYEFENVPFHSSY |
| | | AHSQSLDRLMNPLIDQYLYYLSKTINGSGQNQQTLKFSVAGPSNMAVQGRNYIPGP |
| | | SYRQQRVSTTVTQNNNSEFAWPGASSWALNGRNSLMNPGPAMASHKEGEDRFFPL |
| | | SGSLIFGKQGTGRDNVDADKVMITNEEEIKTTNPVATESYGQVATNHQSAQAQAQT |
| | | GWVQNQGILPGMVWQDRDVYLQGPIWAKIPHTDGNFHPSPLMGGFGMKHPPPQIL |
| | | IKNTPVPADPPTAFNKDKLNSFITQYSTGQVSVEIEWELQKENSKRWNPEIQYTSNY |
| | | YKSNNVEFAVNTEGVYSEPRPIGTRYLTRNL |
| 17 | 3′ UTR | TTGCTTGTTAATCAATAAACCGTTTAATTCGTTTCAGTTGAACTTTGGTCTCTGC |
| | | GTATTTCTTTCTTATCTAGTTTCCATGCTCTAG |
| 18 | poly A | CTGTGCCTTCTAGTTGCCAGCCATCTGTTGTTTGCCCCTCCCCCGTGCCTTCCTTG |
| | | ACCCTGGAAGGTGCCACTCCCACTGTCCTTTCCTAATAAAATGAGGAAATTGCA |
| | | TCGCATTGTCTGAGTAGGTGTCATTCTATTCTGGGGGGTGGGGTGGGGCAGGAC |
| | | AGCAAGGGGGAGGATTGGGAAGACAATAGCAGGCATGCTGGGGA |
| 19 | ITR-R | AGGAACCCCTAGTGATGGAGTTGGCCACTCCCTCTCTGCGCGCTCGCTCGCTCA |
| | | CTGAGGCCGGGCGACCAAAGGTCGCCCGACGCCCGGGCTTTGCCCGGGCGGCC |
| | | TCAGTGAGCGAGCGAGCGCGCAGAGAGGGAGTGGCCAA |

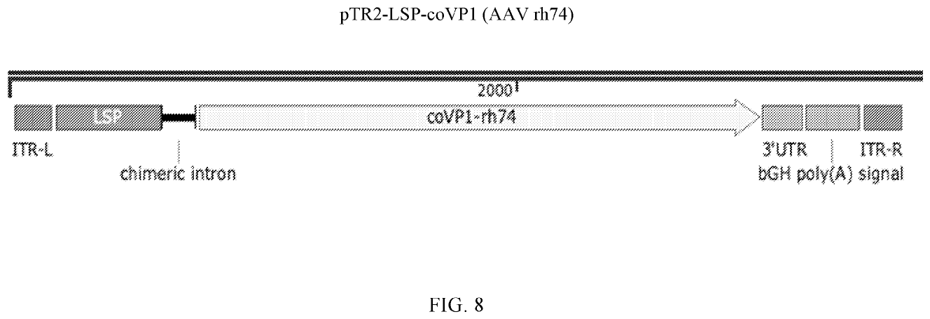

TABLE 3
Construct 3 (pTR2-LSP-coVP1 (AAV rh74); FIG. 8)

| SEQ | | |
|---|---|---|
| ID | Elements | |
| NO: | (5′→3′) | Sequence |
| 13 | ITR-L | TTGGCCACTCCCTCTCTGCGCGCTCGCTCGCTCACTGAGGCCGGGCGACCAAAG |
| | | GTCGCCCGACGCCCGGGCTTTGCCCGGGCGGCCTCAGTGAGCGAGCGAGCGCG |
| | | CAGAGAGGGAGTGGCCAACTCCATCACTAGGGGTTCCT |
| 3 | LSP | CCCTAAAATGGGCAAACATTGCAAGCAGCAAACAGCAAACACACAGCCCTCCC |
| | promoter | TGCCTGCTGACCTTGGAGCTGGGGCAGAGGTCAGAGACCTCTCTGGGGACTGTC |
| | | CCAGGTCAGTGGTGGTGCCTGAAGCTGAGGAGACAGGGCCCTGTCCTCGTCCGT |
| | | ATTTAAGCAGTGGATCCAGAGGGGCAACGGGGGAGGCTGCTGGTGAATATTAA |
| | | CCAAGGTCACCCCAGTTATCGGAGGAGCAAACAGGGGCTAAGTCCACTGGCTG |
| | | GGATCTGAGTCGCCCGCCTACGCTGCCCGGACGCTTTGCCTGGGCAGTGTACAG |
| | | CTTCCACTGCACTTACCGAAAGGAGTCATTGTAGGGCCCTGTCTCCTCAGCTTCA |
| | | GGCACCACCACTGACCTGGGACAGTGAATCCGGA |
| 14 | sd/sa | GTAAGTATCAAGGTTACAAGACAGGTTTAAGGAGACCAATAGAAACTGGGCTT |
| | | GTCGAGACAGAGAAGACTCTTGCGTTTCTGATAGGCACCTATTGGTCTTACTGA |
| | | CATCCACTTTGCCTTTCTCTCCACAG |
| 22 | AAVrh74 | ATGGCGGCTGATGGCTATCTGCCTGATTGGCTGGAAGATAATCTGAGTGAGGGA |
| | coVP1 | ATCCGGGAATGGTGGGACCTGAAACCTGGCGCCCCTAAGCCCAAGGCCAATCA |
| | | GCAGAAACAGGACAATGGCAGAGGCCTGGTGCTGCCAGGCTACAAATACCTTG |
| | | GCCCTTTCAATGGCCTGGATAAGGGCGAGCCAGTGAACGCCGCTGATGCTGCCG |
| | | CTCTGGAACACGACAAGGCCTACGACCAGCAACTGCAGGCCGGTGACAACCCT |
| | | TACCTGAGATACAATCACGCCGATGCCGAGTTCCAGGAGAGACTGCAGGAGGA |
| | | CACCAGCTTCGGAGGCAACCTGGGAAGAGCCGTGTTCCAGGCTAAGAAGAGAG |
| | | TGCTGGAGCCTCTAGGACTAGTGGAAAGCCCAGTGAAGACAGCCCCTGGAAAG |
| | | AAAAGACCCGTGGAACCTTCCCCTCAGAGAAGCCCCGACAGCTCCACCGGCATC |
| | | GGAAAAAAGGGCCAGCAGCCTGCCAAGAAGCGCCTCAACTTCGGACAGACCGG |
| | | CGATAGCGAGTCTGTCCCCGATCCTCAGCCTATTGGCGAACCTCCCGCCGGACC |
| | | TAGCGGCCTGGGCTCTGGCACCATGGCCGCCGGCGGAGGCGCCCCTATGGCTGA |
| | | CAACAACGAGGGCGCTGACGGCGTTGGCAGCTCTTCTGGCAACTGGCACTGCGA |
| | | CTCGACATGGCTGGGCGACCGGGTGATCACAACCAGCACAAGAACCTGGGCCC |
| | | TGCCCACCTACAACAACCACCTGTACAAGCAGATCAGCAATGGCACCTCTGGCG |
| | | GCTCTACAAACGACAACACCTATTTCGGCTATTCTACACCTTGGGGCTACTTCGA |
| | | CTTCAACAGATTTCATTGTCACTTCAGCCCTCGGGACTGGCAGCGGCTGATCAA |
| | | CAACAACTGGGGTTTCCGGCCTAAGCGGCTGAACTTTAAGCTGTTCAACATCCA |
| | | GGTCAAAGAGGTGACACAGAATGAGGGCACCAAGACCATCGCCAACAATCTGA |
| | | CCAGCACCATCCAAGTCTTCACCGACAGCGAGTACCAGCTGCCGTACGTGCTGG |
| | | GCTCCGCCCACCAGGGTTGTCTGCCTCCTTTTCCAGCCGACGTGTTTATGATCCC |
| | | TCAGTATGGCTACCTGACCCTGAACAACGGCTCTCAGGCTGTGGGCCGGAGCAG |
| | | CTTCTACTGCCTGGAATACTTCCCTAGCCAAATGCTGCGGACCGGCAACAACTT |
| | | CGAGTTCAGCTACAATTTCGAGGACGTGCCCTTCCACAGCTCCTACGCCCACAG |
| | | CCAGAGCCTGGACAGACTGATGAACCCCCTTATCGACCAGTACCTCTACTACCT |
| | | GAGCAGAACCCAAAGCACCGGCGGAACCGCCGGCACCCAGCAGCTGCTGTTTT |
| | | CCCAGGCCGGCCCCAACAACATGAGCGCCCAGGCCAAGAACTGGCTGCCTGGC |
| | | CCCTGCTACCGGCAGCAAAGAGTGTCCACAACCCTGTCCCAGAACAACAATTCT |
| | | AATTTTGCCTGGACCGGCGCCACAAAGTACCACCTGAACGGCAGGGACAGCCT |
| | | GGTGAACCCCGGCGTGGCCATGGCCACCCACAAGGATGATGAGGAAAGATTCT |
| | | TCCCTTCTAGCGGCGTCCTGATGTTCGGCAAACAGGGAGCCGGCAAGGACAACG |
| | | TGGACTACAGCAGTGTGATGCTGACATCTGAGGAAGAAATTAAGACCACCAAC |
| | | CCCGTGGCTACAGAACAGTACGGAGTGGTGGCCGACAACCTGCAGCAGCAAAA |
| | | CGCCGCCCCTATCGTTGGAGCCGTGAACAGCCAGGGCGCTCTGCCCGGCATGGT |
| | | GTGGCAGAACAGAGATGTATACCTGCAGGGCCCTATCTGGGCCAAGATCCCCCA |
| | | CACCGATGGCAACTTCCATCCTTCCCCTCTGATGGGCGGATTTGGACTGAAGCA |
| | | CCCCCCTCCACAAATCCTGATCAAGAACACCCCTGTGCCAGCCGACCCTCCTAC |
| | | CACATTCAACCAAGCCAAACTGGCCAGCTTTATCACCCAGTACAGCACAGGCCA |
| | | GGTGTCCGTGGAAATCGAGTGGGAGCTGCAGAAGGAAAACAGCAAGAGATGGA |
| | | ACCCAGAGATCCAGTACACCAGCAACTACTACAAGAGCACGAACGTGGACTTC |
| | | GCCGTGAACACAGAGGGCACATACAGCGAGCCAAGGCCTATCGGCACCAGATA |
| | | CCTGACAAGAAATCTGTGA |
| 23 | AAVrh74 | MAADGYLPDWLEDNLSEGIREWWDLKPGAPKPKANQQKQDNGRGLVLPGYKYL |
| | coVP1 | GPFNGLDKGEPVNAADAAALEHDKAYDQQLQAGDNPYLRYNHADAEFQERLQED |
| | | TSFGGNLGRAVFQAKKRVLEPLGLVESPVKTAPGKKRPVEPSPQRSPDSSTGIGKKG |
| | | QQPAKKRLNFGQTGDSESVPDPQPIGEPPAGPSGLGSGTMAAGGGAPMADNNEGA |
| | | DGVGSSSGNWHCDSTWLGDRVITTSTRTWALPTYNNHLYKQISNGTSGGSTNDNT |
| | | YFGYSTPWGYFDFNRFHCHFSPRDWQRLINNNWGFRPKRLNFKLFNIQVKEVTQNE |
| | | GTKTIANNLTSTIQVFTDSEYQLPYVLGSAHQGCLPPFPADVFMIPQYGYLTLNNGS |
| | | QAVGRSSFYCLEYFPSQMLRTGNNFEFSYNFEDVPFHSSYAHSQSLDRLMNPLIDQY |
| | | LYYLSRTQSTGGTAGTQQLLFSQAGPNNMSAQAKNWLPGPCYRQQRVSTTLSQNN |
| | | NSNFAWTGATKYHLNGRDSLVNPGVAMATHKDDEERFFPSSGVLMFGKQGAGKD |
| | | NVDYSSVMLTSEEEIKTTNPVATEQYGVVADNLQQQNAAPIVGAVNSQGALPGMV |
| | | WQNRDVYLQGPIWAKIPHTDGNFHPSPLMGGFGLKHPPPQILIKNTPVPADPPTTFN |
| | | QAKLASFITQYSTGQVSVEIEWELQKENSKRWNPEIQYTSNYYKSTNVDFAVNTEG |
| | | TYSEPRPIGTRYLTRNL |
| 24 | 3′ UTR | TTACATGTTAATCAATAAACCGGTTAATTCGTTTCAGTTGAACTTTGGTCTCCTG |
| | | TCCTTCTTATCTTATCGGTTACCATAGAAACTGGTTACTTATTAACTGCTTGGTG |
| | | CGCTTCGCGATAAAAGACTTACGTCATCGGGTTACCCCTAGTGATGGA |
| 18 | poly A | CTGTGCCTTCTAGTTGCCAGCCATCTGTTGTTTGCCCCTCCCCCGTGCCTTCCTTG |
| | | ACCCTGGAAGGTGCCACTCCCACTGTCCTTTCCTAATAAAATGAGGAAATTGCA |
| | | TCGCATTGTCTGAGTAGGTGTCATTCTATTCTGGGGGGTGGGGTGGGGCAGGAC |
| | | AGCAAGGGGGAGGATTGGGAAGACAATAGCAGGCATGCTGGGGA |
| 19 | ITR-R | AGGAACCCCTAGTGATGGAGTTGGCCACTCCCTCTCTGCGCGCTCGCTCGCTCA |
| | | CTGAGGCCGGGCGACCAAAGGTCGCCCGACGCCCGGGCTTTGCCCGGGCGGCC |
| | | TCAGTGAGCGAGCGAGCGCGCAGAGAGGGAGTGGCCAA |

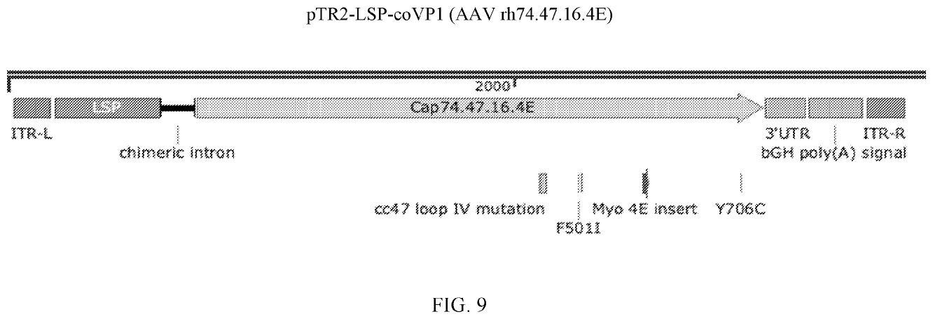

TABLE 4
Construct 4 (pTR2-LSP-coVP1 (AAV rh74.47.4E); FIG. 9)

| SEQ | | |
|---|---|---|
| ID | Elements | |
| NO: | (5′→3′) | Sequence |
| 13 | ITR-L | TTGGCCACTCCCTCTCTGCGCGCTCGCTCGCTCACTGAGGCCGGGCGACCAAAG |
| | | GTCGCCCGACGCCCGGGCTTTGCCCGGGCGGCCTCAGTGAGCGAGCGAGCGCG |
| | | CAGAGAGGGAGTGGCCAACTCCATCACTAGGGGTTCCT |
| 3 | LSP | CCCTAAAATGGGCAAACATTGCAAGCAGCAAACAGCAAACACACAGCCCTCCC |
| | promoter | TGCCTGCTGACCTTGGAGCTGGGGCAGAGGTCAGAGACCTCTCTGGGGACTGTC |
| | | CCAGGTCAGTGGTGGTGCCTGAAGCTGAGGAGACAGGGCCCTGTCCTCGTCCGT |
| | | ATTTAAGCAGTGGATCCAGAGGGGCAACGGGGGAGGCTGCTGGTGAATATTAA |
| | | CCAAGGTCACCCCAGTTATCGGAGGAGCAAACAGGGGCTAAGTCCACTGGCTG |
| | | GGATCTGAGTCGCCCGCCTACGCTGCCCGGACGCTTTGCCTGGGCAGTGTACAG |
| | | CTTCCACTGCACTTACCGAAAGGAGTCATTGTAGGGCCCTGTCTCCTCAGCTTCA |
| | | GGCACCACCACTGACCTGGGACAGTGAATCCGGA |
| 14 | sd/sa | GTAAGTATCAAGGTTACAAGACAGGTTTAAGGAGACCAATAGAAACTGGGCTT |
| | | GTCGAGACAGAGAAGACTCTTGCGTTTCTGATAGGCACCTATTGGTCTTACTGA |
| | | CATCCACTTTGCCTTTCTCTCCACAG |
| 25 | AAVrh74. | ATGGCTGCCGATGGTTATCTTCCAGATTGGCTCGAGGACAACCTCTCTGAGGGC |
| | 47.16.4E | ATTCGCGAGTGGTGGGACCTGAAACCTGGAGCCCCGAAACCCAAAGCCAACCA |
| | coVP1 | GCAAAAGCAGGACAACGGCCGGGGTCTGGTGCTTCCTGGCTACAAGTACCTCG |
| | | GACCCTTCAACGGACTCGACAAGGGGGAGCCCGTCAACGCGGCGGACGCAGCG |
| | | GCCCTCGAGCACGACAAGGCCTACGACCAGCAGCTCCAAGCGGGTGACAATCC |
| | | GTACCTGCGGTATAATCACGCCGACGCCGAGTTTCAGGAGCGTCTGCAAGAAGA |
| | | TACGTCTTTTGGGGGCAACCTCGGGCGCGCAGTCTTCCAGGCCAAAAAGCGGGT |
| | | TCTCGAACCTCTGGGCCTGGTTGAATCGCCGGTTAAGACGGCTCCTGGAAAGAA |
| | | GAGACCGGTAGAGCCATCACCCCAGCGCTCTCCAGACTCCTCTACGGGCATCGG |
| | | CAAGAAAGGCCAGCAGCCCGCAAAAAAGAGACTCAATTTTGGGCAGACTGGCG |
| | | ACTCAGAGTCAGTCCCCGACCCTCAACCAATCGGAGAACCACCAGCAGGCCCCT |
| | | CTGGTCTGGGATCTGGTACAATGGCTGCAGGCGGTGGCGCTCCAATGGCAGACA |
| | | ATAACGAAGGCGCCGACGGAGTGGGTAGTTCCTCAGGAAATTGGCATTGCGATT |
| | | CCACATGGCTGGGCGACAGAGTCATCACCACCAGCACCCGCACCTGGGCCCTGC |
| | | CCACCTACAACAACCACCTCTACAAGCAAATCTCCAACGGGACCTCGGGAGGA |
| | | AGCACCAACGACAACACCTACTTCGGCTACAGCACCCCCTGGGGGTATTTTGAC |
| | | TTCAACAGATTCCACTGCCACTTTTCACCACGTGACTGGCAGCGACTCATCAAC |
| | | AACAACTGGGGATTCCGGCCCAAGAGGCTCAACTTCAAGCTCTTCAACATCCAA |
| | | GTCAAGGAGGTCACGCAGAATGAAGGCACCAAGACCATCGCCAATAACCTTAC |
| | | CAGCACGATTCAGGTCTTTACGGACTCGGAATACCAGCTCCCGTACGTGCTCGG |
| | | CTCGGCGCACCAGGGCTGCCTGCCTCCGTTCCCGGCGGACGTCTTCATGATTCCT |
| | | CAGTACGGGTACCTGACTCTGAACAATGGCAGTCAGGCTGTGGGCCGGTCGTCC |
| | | TTCTACTGCCTGGAGTACTTTCCTTCTCAAATGCTGAGAACGGGCAACAACTTTG |
| | | AATTCAGCTACAACTTCGAGGACGTGCCCTTCCACAGCAGCTACGCGCACAGCC |
| | | AGAGCCTGGACCGGCTGATGAACCCTCTCATCGACCAGTACTTGTACTACCTGT |
| | | CCCGGACTATCGGAGTGTCCCTCGGAGGAGGACAGCAGTTGCTATTTTCTCAGG |
| | | CCGGGCCTAACAACATGTCGGCTCAGGCCAAGAACTGGCTACCCGGTCCCTGCT |
| | | ACCGGCAGCAACGCGTCTCCACGACACTGTCGCAGAACAACAACAGCAACATC |
| | | GCCTGGACGCGGGCCACCAAGTATCATCTGAATGGCAGAGACTCTCTGGTGAAT |
| | | CCTGGCGTTGCCATGGCTACCCACAAGGACGACGAAGAGCGATTTTTTCCATCC |
| | | AGCGGAGTCTTAATGTTTGGGAAACAGGGAGCTGGAAAAGACAACGTGGACTA |
| | | TAGCAGCGTGATGCTAACCAGCGAGGAAGAAATAAAGACCACCAACCCAGTGG |
| | | CCACAGAACAGTACGGCGTGGTGGCCGATAACCTGCAAGAGAACAGAAGAGGC |
| | | GACTTCAACAACCAGCAAAACGCCGCTCCTATTGTAGGGGCCGTCAATAGTCAA |
| | | GGAGCCTTACCTGGCATGGTGTGGCAGAACCGGGACGTGTACCTGCAGGGTCCC |
| | | ATCTGGGCCAAGATTCCTCATACGGACGGCAACTTTCATCCCTCGCCGCTGATG |
| | | GGAGGCTTTGGACTGAAGCATCCGCCTCCTCAGATCCTGATTAAAAACACACCT |
| | | GTTCCCGCGGATCCTCCGACCACCTTCAATCAGGCCAAGCTGGCTTCTTTCATCA |
| | | CGCAGTACAGTACCGGCCAGGTCAGCGTGGAGATCGAGTGGGAGCTGCAGAAG |
| | | GAGAACAGCAAACGCTGGAACCCAGAGATTCAGTACACTTCCAACTGCTACAA |
| | | ATCTACAAATGTGGACTTTGCTGTCAATACTGAGGGTACTTATTCCGAGCCTCGC |
| | | CCCATTGGCACCCGTTACCTCACCCGTAATCTGTAA |
| 26 | AAVrh74. | MAADGYLPDWLEDNLSEGIREWWDLKPGAPKPKANQQKQDNGRGLVLPGYKYL |
| | 47.16.4E | GPFNGLDKGEPVNAADAAALEHDKAYDQQLQAGDNPYLRYNHADAEFQERLQED |
| | coVP1 | TSFGGNLGRAVFQAKKRVLEPLGLVESPVKTAPGKKRPVEPSPQRSPDSSTGIGKKG |
| | | QQPAKKRLNFGQTGDSESVPDPQPIGEPPAGPSGLGSGTMAAGGGAPMADNNEGA |
| | | DGVGSSSGNWHCDSTWLGDRVITTSTRTWALPTYNNHLYKQISNGTSGGSTNDNT |
| | | YFGYSTPWGYFDFNRFHCHFSPRDWQRLINNNWGFRPKRLNFKLFNIQVKEVTQNE |
| | | GTKTIANNLTSTIQVFTDSEYQLPYVLGSAHQGCLPPFPADVFMIPQYGYLTLNNGS |
| | | QAVGRSSFYCLEYFPSQMLRTGNNFEFSYNFEDVPFHSSYAHSQSLDRLMNPLIDQY |
| | | LYYLSRTIGVSLGGGQQLLFSQAGPNNMSAQAKNWLPGPCYRQQRVSTTLSQNNN |
| | | SNIAWTRATKYHLNGRDSLVNPGVAMATHKDDEERFFPSSGVLMFGKQGAGKDN |
| | | VDYSSVMLTSEEEIKTTNPVATEQYGVVADNLQENRRGDFNNQQNAAPIVGAVNS |
| | | QGALPGMVWQNRDVYLQGPIWAKIPHTDGNFHPSPLMGGFGLKHPPPQILIKNTPV |
| | | PADPPTTFNQAKLASFITQYSTGQVSVEIEWELQKENSKRWNPEIQYTSNCYKSTNV |
| | | DFAVNTEGTYSEPRPIGTRYLTRNL |
| 24 | 3′ UTR | TTACATGTTAATCAATAAACCGGTTAATTCGTTTCAGTTGAACTTTGGTCTCCTG |
| | | TCCTTCTTATCTTATCGGTTACCATAGAAACTGGTTACTTATTAACTGCTTGGTG |
| | | CGCTTCGCGATAAAAGACTTACGTCATCGGGTTACCCCTAGTGATGGA |
| 18 | poly A | CTGTGCCTTCTAGTTGCCAGCCATCTGTTGTTTGCCCCTCCCCCGTGCCTTCCTTG |
| | | ACCCTGGAAGGTGCCACTCCCACTGTCCTTTCCTAATAAAATGAGGAAATTGCA |
| | | TCGCATTGTCTGAGTAGGTGTCATTCTATTCTGGGGGGTGGGGTGGGGCAGGAC |
| | | AGCAAGGGGGAGGATTGGGAAGACAATAGCAGGCATGCTGGGGA |
| 19 | ITR-R | AGGAACCCCTAGTGATGGAGTTGGCCACTCCCTCTCTGCGCGCTCGCTCGCTCA |
| | | CTGAGGCCGGGCGACCAAAGGTCGCCCGACGCCCGGGCTTTGCCCGGGCGGCC |
| | | TCAGTGAGCGAGCGAGCGCGCAGAGAGGGAGTGGCCAA |

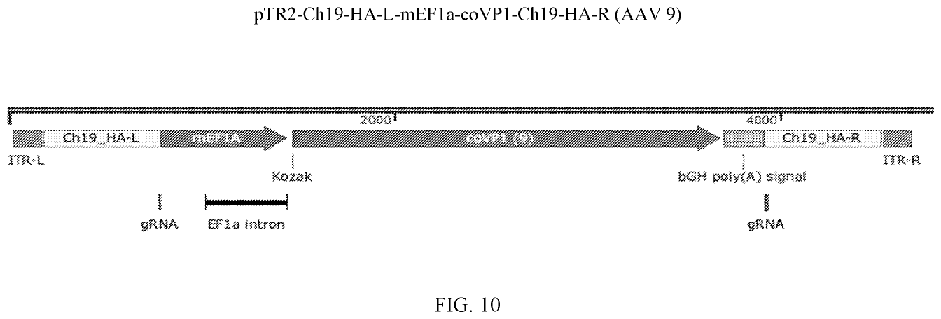

TABLE 5
Construct 5 (pTR2-CH19_HA-L-mEF1a-coVP1-HA-R (AAV9); FIG. 10)

| SEQ | | |
|---|---|---|
| ID | Elements | |
| NO: | (5′→3′) | Sequence |
| 13 | ITR-L | TTGGCCACTCCCTCTCTGCGCGCTCGCTCGCTCACTGAGGCCGGGCGACCAAAG |
| | | GTCGCCCGACGCCCGGGCTTTGCCCGGGCGGCCTCAGTGAGCGAGCGAGCGCG |
| | | CAGAGAGGGAGTGGCCAACTCCATCACTAGGGGTTCCT |
| 27 | Ch19-HA- | CACCACGTGATGTCCTCTGAGCGGATCCTCCCCGTGTCTGGGTCCTCTCCGGGCA |
| | L | TCTCTCCTCCCTCACCCAACCCCATGCCGTCTTCACTCGCTGGGTTCCCTTTTCCT |
| | | TCTCCTTCTGGGGCCTGTGCCATCTCTCGTTTCTTAGGATGGCCTTCTCCGACGG |
| | | ATGTCTCCCTTGCGTCCCGCCTCCCCTTCTTGTAGGCCTGCATCATCACCGTTTTT |
| | | CTGGACAACCCCAAAGTACCCCGTCTCCCTGGCTTTAGCCACCTCTCCATCCTCT |
| | | TGCTTTCTTTGCCTGGACACCCCGTTCTCCTGTGGATTCGGGTCACCTCTCACTC |
| | | CTTTCATTTGGGCAGCTCCCCTACCCCCCTTACCTCTCTAGTCTGTGCTAGCTCTT |
| | | CCAGCCCCCTGTCATGGCATCTTCCAGGGGTCCGAGAGCTCAGCTAGTCTTCTTC |
| | | CTCCAACCCGGGCCCCTATGTCCACTTCAGGACAGCATGTTTGCTGCCTCCAGG |
| | | GATCCTGTGTCCCCGAGCTGGGACCACCTTATATTCCCAGGGCCGGTTAATGTG |
| | | GCTCTGGTTCTGGGTACTTTTATCTGTCCCCTCCACCCCACAGTGGGGC |
| 28 | mEF1a | GGCTCCGGTGCCCGTCAGTGGGCAGAGCGCACATCGCCCACAGTCCCCGAGAA |
| | | GTTGGGGGGAGGGGTCGGCAATTGAACCGGTGCCTAGAGAAGGTGGCGCGGGG |
| | | TAAACTGGGAAAGTGATGTCGTGTACTGGCTCCGCCTTTTTCCCGAGGGTGGGG |
| | | GAGAACCGTATATAAGTGCAGTAGTCGCCGTGAACGTTCTTTTTCGCAACGGGT |
| | | TTGCCGCCAGAACACAGGTAAGTGCCGTGTGTGGTTCCCGCGGGCCTGGCCTCT |
| | | TTACGGGTTATGGCCCTTGCGTGCCTTGAATTACTTCCACCTGGCTGCAGTACGT |
| | | GATTCTTGATCCCGAGCTTCGGGTTGGAAGTGGGTGGGAGAGTTCGAGGCCTTG |
| | | CGCTTAATCAGCCGTCGCTTCATGTGACTCCACGGAGTACCGGGCGCCGTCCAG |
| | | GCACCTCGATTAGTTCTCGAGCTTTTGGAGTACGTCGTCTTTAGGTTGGGGGGA |
| | | GGGGTTTTATGCGATGGAGTTTCCCCACACTGAGTGGGTGGAGACTGAAGTTAG |
| | | GCCAGCTTGGCACTTGATGTAATTCTCCTTGGAATTTGCCCTTTTTGAGTTTGGA |
| | | TCTTGGTTCATTCTCAAGCCTCAGACAGTGGTTCAAAGTTTTTTTCTTCCATTTCA |
| | | GGTGTCGTGA |
| 29 | AAV9 | ATGGCTGCCGATGGTTATCTTCCAGATTGGCTCGAGGACAACCTTAGTGAAGGA |
| | coVP1 | ATTCGCGAGTGGTGGGCTTTGAAACCTGGAGCCCCTCAACCCAAGGCAAATCAA |
| | | CAACATCAAGACAACGCTCGAGGTCTTGTGCTTCCGGGTTACAAATACCTTGGA |
| | | CCCGGCAACGGACTCGACAAGGGGGAGCCGGTCAACGCAGCAGACGCGGCGGC |
| | | CCTCGAGCACGACAAGGCCTACGACCAGCAGCTCAAGGCCGGAGACAACCCGT |
| | | ACCTCAAGTACAACCACGCCGACGCCGAGTTCCAGGAGCGGCTCAAAGAAGAT |
| | | ACGTCTTTTGGGGGCAACCTCGGGCGAGCAGTCTTCCAGGCCAAAAAGAGGCTT |
| | | CTTGAACCTCTTGGTCTGGTTGAGGAAGCGGCTAAGACGGCTCCTGGAAAGAAG |
| | | AGGCCTGTAGAGCAGTCTCCTCAGGAACCGGACTCCTCCGCGGGTATTGGCAAA |
| | | TCGGGTGCACAGCCCGCTAAAAAGAGACTCAATTTCGGTCAGACTGGCGACAC |
| | | AGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAA |
| | | CGAAGGTGCCGATGGAGTGGGTAGTTCCTCGGGAAATTGGCATTGCGATTCCCA |
| | | ATGGCTGGGGGACAGAGTCATCACCACCAGCACCCGAACCTGGGCCCTGCCCA |
| | | CCTACAACAATCACCTCTACAAGCAAATCTCCAACAGCACATCTGGAGGATCTT |
| | | CAAATGACAACGCCTACTTCGGCTACAGCACCCCCTGGGGGTATTTTGACTTCA |
| | | ACAGATTCCACTGCCACTTCTCACCACGTGACTGGCAGCGACTCATCAACAACA |
| | | ACTGGGGATTCCGGCCTAAGCGACTCAACTTCAAGCTCTTCAACATTCAGGTCA |
| | | AAGAGGTTACGGACAACAATGGAGTCAAGACCATCGCCAATAACCTTACCAGC |
| | | ACGGTCCAGGTCTTCACGGACTCAGACTATCAGCTCCCGTACGTGCTCGGGTCG |
| | | GCTCACGAGGGCTGCCTCCCGCCGTTCCCAGCGGACGTTTTCATGATTCCTCAGT |
| | | ACGGGTATCTGACGCTTAATGATGGAAGCCAGGCCGTGGGTCGTTCGTCCTTTT |
| | | ACTGCCTGGAATATTTCCCGTCGCAAATGCTAAGAACGGGTAACAACTTCCAGT |
| | | TCAGCTACGAGTTTGAGAACGTACCTTTCCATAGCAGCTACGCTCACAGCCAAA |
| | | GCCTGGACCGACTAATGAATCCACTCATCGACCAATACTTGTACTATCTCTCAA |
| | | AGACTATTAACGGTTCTGGACAGAATCAACAAACGCTAAAATTCAGTGTGGCCG |
| | | GACCCAGCAACATGGCTGTCCAGGGAAGAAACTACATACCTGGACCCAGCTAC |
| | | CGACAACAACGTGTCTCAACCACTGTGACTCAAAACAACAACAGCGAATTTGCT |
| | | TGGCCTGGAGCTTCTTCTTGGGCTCTCAATGGACGTAATAGCTTGATGAATCCTG |
| | | GACCTGCTATGGCCAGCCACAAAGAAGGAGAGGACCGTTTCTTTCCTTTGTCTG |
| | | GATCTTTAATTTTTGGCAAACAAGGAACTGGAAGAGACAACGTGGATGCGGAC |
| | | AAAGTCATGATAACCAACGAAGAAGAAATTAAAACTACTAACCCGGTAGCAAC |
| | | GGAGTCCTATGGACAAGTGGCCACAAACCACCAGAGTGCCCAAGCACAGGCGC |
| | | AGACCGGCTGGGTTCAAAACCAAGGAATACTTCCGGGTATGGTTTGGCAGGAC |
| | | AGAGATGTGTACCTGCAAGGACCCATTTGGGCCAAAATTCCTCACACGGACGGC |
| | | AACTTTCACCCTTCTCCGCTGATGGGAGGGTTTGGAATGAAGCACCCGCCTCCT |
| | | CAGATCCTCATCAAAAACACACCTGTACCTGCGGATCCTCCAACGGCCTTCAAC |
| | | AAGGACAAGCTGAACTCTTTCATCACCCAGTATTCTACTGGCCAAGTCAGCGTG |
| | | GAGATCGAGTGGGAGCTGCAGAAGGAAAACAGCAAGCGCTGGAACCCGGAGA |
| | | TCCAGTACACTTCCAACTATTACAAGTCTAATAATGTTGAATTTGCTGTTAATAC |
| | | TGAAGGTGTATATAGTGAACCCCGCCCCATTGGCACCAGATACCTGACTCGTAA |
| | | TCTGTAA |
| 16 | AAV9 | MAADGYLPDWLEDNLSEGIREWWALKPGAPQPKANQQHQDNARGLVLPGYKYL |
| | coVP1 | GPGNGLDKGEPVNAADAAALEHDKAYDQQLKAGDNPYLKYNHADAEFQERLKE |
| | | DTSFGGNLGRAVFQAKKRLLEPLGLVEEAAKTAPGKKRPVEQSPQEPDSSAGIGKS |
| | | GAQPAKKRLNFGQTGDTESVPDPQPIGEPPAAPSGVGSLTMASGGGAPVADNNEGA |
| | | DGVGSSSGNWHCDSQWLGDRVITTSTRTWALPTYNNHLYKQISNSTSGGSSNDNA |
| | | YFGYSTPWGYFDFNRFHCHFSPRDWQRLINNNWGFRPKRLNFKLFNIQVKEVTDN |
| | | NGVKTIANNLTSTVQVFTDSDYQLPYVLGSAHEGCLPPFPADVFMIPQYGYLTLND |
| | | GSQAVGRSSFYCLEYFPSQMLRTGNNFQFSYEFENVPFHSSYAHSQSLDRLMNPLID |
| | | QYLYYLSKTINGSGQNQQTLKFSVAGPSNMAVQGRNYIPGPSYRQQRVSTTVTQNN |
| | | NSEFAWPGASSWALNGRNSLMNPGPAMASHKEGEDRFFPLSGSLIFGKQGTGRDN |
| | | VDADKVMITNEEEIKTTNPVATESYGQVATNHQSAQAQAQTGWVQNQGILPGMV |
| | | WQDRDVYLQGPIWAKIPHTDGNFHPSPLMGGFGMKHPPPQILIKNTPVPADPPTAF |
| | | NKDKLNSFITQYSTGQVSVEIEWELQKENSKRWNPEIQYTSNYYKSNNVEFAVNTE |
| | | GVYSEPRPIGTRYLTRNL |
| 30 | HA-R | CTGTGCCTTCTAGTTGCCAGCCATCTGTTGTTTGCCCCTCCCCCGTGCCTTCCTTG |
| | | ACCCTGGAAGGTGCCACTCCCACTGTCCTTTCCTAATAAAATGAGGAAATTGCA |
| | | TCGCATTGTCTGAGTAGGTGTCATTCTATTCTGGGGGGTGGGGGGGGCAGGAC |
| | | AGCAAGGGGGAGGATTGGGAAGACAATAGCAGGCATGCTGGGGA |
| 31 | Ch19-HA- | ACTAGGGACAGGATTGGTGACAGAAAAGCCCCATCCTTAGGCCTCCTCCTTCCT |
| | R | AGTCTCCTGATATTGGGTCTAACCCCCACCTCCTGTTAGGCAGATTCCTTATCTG |
| | | GTGACACACCCCCATTTCCTGGAGCCATCTCTCTCCTTGCCAGAACCTCTAAGGT |
| | | TTGCTTACGATGGAGCCAGAGAGGATCCTGGGAGGGAGAGCTTGGCAGGGGGT |
| | | GGGAGGGAAGGGGGGGATGCGTGACCTGCCCGGTTCTCAGTGGCCACCCTGCG |
| | | CTACCCTCTCCCAGAACCTGAGCTGCTCTGACGCGGCTGTCTGGTGCGTTTCACT |
| | | GATCCTGGTGCTGCAGCTTCCTTACACTTCCCAAGAGGAGAAGCAGTTTGGAAA |
| | | AACAAAATCAGAATAAGTTGGTCCTGAGTTCTAACTTTGGCTCTTCACCTTTCTA |
| | | GTCCCCAATTTATATTGTTCCTCCGTGCGTCAGTTTTACCTGTGAGATAAGGCCA |
| | | GTAGCCAGCCCCGTCCTGGCAGGGCTGTGGTGAGGAGGGGGGTGTCCGTGTGG |
| | | AAAACTCCCTTTGTGAGAATGGTGCGTCCTAGGTGTTCACCAGGTCGTGGCCGC |
| | | CTCT |
| 19 | ITR-R | AGGAACCCCTAGTGATGGAGTTGGCCACTCCCTCTCTGCGCGCTCGCTCGCTCA |
| | | CTGAGGCCGGGCGACCAAAGGTCGCCCGACGCCCGGGCTTTGCCCGGGCGGCC |
| | | TCAGTGAGCGAGCGAGCGCGCAGAGAGGGAGTGGCCAA |

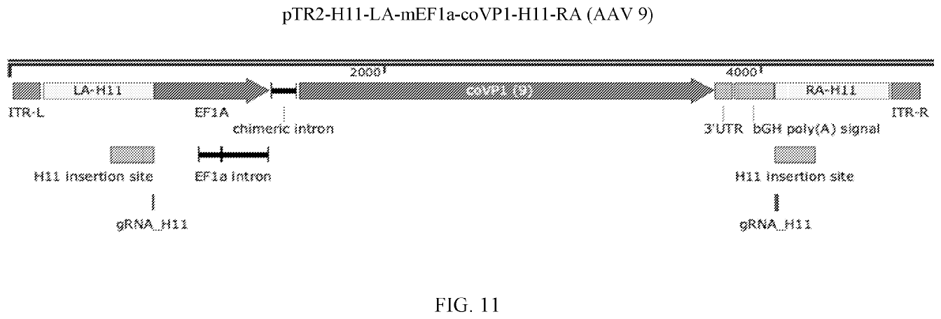

TABLE 6
Construct 5 (pTR2-H11-L-mEF1a-coVP1-H11-R (AAV9); FIG. 11).

| SEQ | | |
|---|---|---|
| ID | Elements | |
| NO: | (5′→3′) | Sequence |
| 13 | ITR-L | TTGGCCACTCCCTCTCTGCGCGCTCGCTCGCTCACTGAGGCCGGGCGACCAAAG |
| | | GTCGCCCGACGCCCGGGCTTTGCCCGGGCGGCCTCAGTGAGCGAGCGAGCGCG |
| | | CAGAGAGGGAGTGGCCAACTCCATCACTAGGGGTTCCT |
| 32 | H11-L | CTCATCTTGTGCCTGCTGGACTTCCACCGTTGTTTCATGTATCTCGTTAGCTGAG |
| | | ATAGCACTTCCTCCTGCCCTTACCCTTTATCTGGCTCTTAGCTCCTGAAAACTGC |
| | | ATTATTAGCTTCCTCTTTTGCCTCTACTCTTACTCAACCAAAATTGTTTTAAGATC |
| | | TGTGGATCTAGCTTCTGCTGTGCTATTCTTAGGAACACTTTTATTTCCTCTTAGCT |
| | | CCATCTCACCAGTTATTGGCTAATGGCTTTGCTTGGTACCTACATCTGTACATTT |
| | | CTTTCGTACTAGCTTCTAGACTGAAAAAGGACTGTTGGTTCAACATGAAAGGGA |
| | | AGGAGGTAAAAGAGGACACACAGGAAAGATGGATTGGGATTCAGGTCTCTGCT |
| | | GTTGTTACTTGAGATTGCTTTCTAGATTCTACTTGTGGAAACAAAAAGCCTTTGC |
| | | GAGAATTCTAAACTGGAGTATTTCTGTAATTGAGGAGTCTTGCTCAGCAAATCC |
| | | CACTTAGGGGACTAATGAAGTACCAGGAAGAGACAGACCATGCTCAATCCACA |
| | | AAGCCAGGTTTTACTGAAATGTGACCTACTTTCTTATGTTCCTG |
| 28 | mEF1a | GGCTCCGGTGCCCGTCAGTGGGCAGAGCGCACATCGCCCACAGTCCCCGAGAA |
| | | GTTGGGGGGAGGGGTCGGCAATTGAACCGGTGCCTAGAGAAGGTGGCGCGGGG |
| | | TAAACTGGGAAAGTGATGTCGTGTACTGGCTCCGCCTTTTTCCCGAGGGTGGGG |
| | | GAGAACCGTATATAAGTGCAGTAGTCGCCGTGAACGTTCTTTTTCGCAACGGGT |
| | | TTGCCGCCAGAACACAGGTAAGTGCCGTGTGTGGTTCCCGCGGGCCTGGCCTCT |
| | | TTACGGGTTATGGCCCTTGCGTGCCTTGAATTACTTCCACCTGGCTGCAGTACGT |
| | | GATTCTTGATCCCGAGCTTCGGGTTGGAAGTGGGTGGGAGAGTTCGAGGCCTTG |
| | | CGCTTAATCAGCCGTCGCTTCATGTGACTCCACGGAGTACCGGGCGCCGTCCAG |
| | | GCACCTCGATTAGTTCTCGAGCTTTTGGAGTACGTCGTCTTTAGGTTGGGGGGA |
| | | GGGGTTTTATGCGATGGAGTTTCCCCACACTGAGTGGGTGGAGACTGAAGTTAG |
| | | GCCAGCTTGGCACTTGATGTAATTCTCCTTGGAATTTGCCCTTTTTGAGTTTGGA |
| | | TCTTGGTTCATTCTCAAGCCTCAGACAGTGGTTCAAAGTTTTTTTCTTCCATTTCA |
| | | GGTGTCGTGA |
| 29 | AAV9 | ATGGCTGCCGATGGTTATCTTCCAGATTGGCTCGAGGACAACCTTAGTGAAGGA |
| | coVP1 | ATTCGCGAGTGGTGGGCTTTGAAACCTGGAGCCCCTCAACCCAAGGCAAATCAA |
| | | CAACATCAAGACAACGCTCGAGGTCTTGTGCTTCCGGGTTACAAATACCTTGGA |
| | | CCCGGCAACGGACTCGACAAGGGGGAGCCGGTCAACGCAGCAGACGCGGCGGC |
| | | CCTCGAGCACGACAAGGCCTACGACCAGCAGCTCAAGGCCGGAGACAACCCGT |
| | | ACCTCAAGTACAACCACGCCGACGCCGAGTTCCAGGAGCGGCTCAAAGAAGAT |
| | | ACGTCTTTTGGGGGCAACCTCGGGCGAGCAGTCTTCCAGGCCAAAAAGAGGCTT |
| | | CTTGAACCTCTTGGTCTGGTTGAGGAAGCGGCTAAGACGGCTCCTGGAAAGAAG |
| | | AGGCCTGTAGAGCAGTCTCCTCAGGAACCGGACTCCTCCGCGGGTATTGGCAAA |
| | | TCGGGTGCACAGCCCGCTAAAAAGAGACTCAATTTCGGTCAGACTGGCGACAC |
| | | AGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAA |
| | | CGAAGGTGCCGATGGAGTGGGTAGTTCCTCGGGAAATTGGCATTGCGATTCCCA |
| | | ATGGCTGGGGGACAGAGTCATCACCACCAGCACCCGAACCTGGGCCCTGCCCA |
| | | CCTACAACAATCACCTCTACAAGCAAATCTCCAACAGCACATCTGGAGGATCTT |
| | | CAAATGACAACGCCTACTTCGGCTACAGCACCCCCTGGGGGTATTTTGACTTCA |
| | | ACAGATTCCACTGCCACTTCTCACCACGTGACTGGCAGCGACTCATCAACAACA |
| | | ACTGGGGATTCCGGCCTAAGCGACTCAACTTCAAGCTCTTCAACATTCAGGTCA |
| | | AAGAGGTTACGGACAACAATGGAGTCAAGACCATCGCCAATAACCTTACCAGC |
| | | ACGGTCCAGGTCTTCACGGACTCAGACTATCAGCTCCCGTACGTGCTCGGGTCG |
| | | GCTCACGAGGGCTGCCTCCCGCCGTTCCCAGCGGACGTTTTCATGATTCCTCAGT |
| | | ACGGGTATCTGACGCTTAATGATGGAAGCCAGGCCGTGGGTCGTTCGTCCTTTT |
| | | ACTGCCTGGAATATTTCCCGTCGCAAATGCTAAGAACGGGTAACAACTTCCAGT |
| | | TCAGCTACGAGTTTGAGAACGTACCTTTCCATAGCAGCTACGCTCACAGCCAAA |
| | | GCCTGGACCGACTAATGAATCCACTCATCGACCAATACTTGTACTATCTCTCAA |
| | | AGACTATTAACGGTTCTGGACAGAATCAACAAACGCTAAAATTCAGTGTGGCCG |
| | | GACCCAGCAACATGGCTGTCCAGGGAAGAAACTACATACCTGGACCCAGCTAC |
| | | CGACAACAACGTGTCTCAACCACTGTGACTCAAAACAACAACAGCGAATTTGCT |
| | | TGGCCTGGAGCTTCTTCTTGGGCTCTCAATGGACGTAATAGCTTGATGAATCCTG |
| | | GACCTGCTATGGCCAGCCACAAAGAAGGAGAGGACCGTTTCTTTCCTTTGTCTG |
| | | GATCTTTAATTTTTGGCAAACAAGGAACTGGAAGAGACAACGTGGATGCGGAC |
| | | AAAGTCATGATAACCAACGAAGAAGAAATTAAAACTACTAACCCGGTAGCAAC |
| | | GGAGTCCTATGGACAAGTGGCCACAAACCACCAGAGTGCCCAAGCACAGGCGC |
| | | AGACCGGCTGGGTTCAAAACCAAGGAATACTTCCGGGTATGGTTTGGCAGGAC |
| | | AGAGATGTGTACCTGCAAGGACCCATTTGGGCCAAAATTCCTCACACGGACGGC |
| | | AACTTTCACCCTTCTCCGCTGATGGGAGGGTTTGGAATGAAGCACCCGCCTCCT |
| | | CAGATCCTCATCAAAAACACACCTGTACCTGCGGATCCTCCAACGGCCTTCAAC |
| | | AAGGACAAGCTGAACTCTTTCATCACCCAGTATTCTACTGGCCAAGTCAGCGTG |
| | | GAGATCGAGTGGGAGCTGCAGAAGGAAAACAGCAAGCGCTGGAACCCGGAGA |
| | | TCCAGTACACTTCCAACTATTACAAGTCTAATAATGTTGAATTTGCTGTTAATAC |
| | | TGAAGGTGTATATAGTGAACCCCGCCCCATTGGCACCAGATACCTGACTCGTAA |
| | | TCTGTAA |
| 16 | AAV9 | MAADGYLPDWLEDNLSEGIREWWALKPGAPQPKANQQHQDNARGLVLPGYKYL |
| | coVP1 | GPGNGLDKGEPVNAADAAALEHDKAYDQQLKAGDNPYLKYNHADAEFQERLKE |
| | | DTSFGGNLGRAVFQAKKRLLEPLGLVEEAAKTAPGKKRPVEQSPQEPDSSAGIGKS |
| | | GAQPAKKRLNFGQTGDTESVPDPQPIGEPPAAPSGVGSLTMASGGGAPVADNNEGA |
| | | DGVGSSSGNWHCDSQWLGDRVITTSTRTWALPTYNNHLYKQISNSTSGGSSNDNA |
| | | YFGYSTPWGYFDFNRFHCHFSPRDWQRLINNNWGFRPKRLNFKLFNIQVKEVTDN |
| | | NGVKTIANNLTSTVQVFTDSDYQLPYVLGSAHEGCLPPFPADVFMIPQYGYLTLND |
| | | GSQAVGRSSFYCLEYFPSQMLRTGNNFQFSYEFENVPFHSSYAHSQSLDRLMNPLID |
| | | QYLYYLSKTINGSGQNQQTLKFSVAGPSNMAVQGRNYIPGPSYRQQRVSTTVTQNN |
| | | NSEFAWPGASSWALNGRNSLMNPGPAMASHKEGEDRFFPLSGSLIFGKQGTGRDN |
| | | VDADKVMITNEEEIKTTNPVATESYGQVATNHQSAQAQAQTGWVQNQGILPGMV |
| | | WQDRDVYLQGPIWAKIPHTDGNFHPSPLMGGFGMKHPPPQILIKNTPVPADPPTAF |
| | | NKDKLNSFITQYSTGQVSVEIEWELQKENSKRWNPEIQYTSNYYKSNNVEFAVNTE |
| | | GVYSEPRPIGTRYLTRNL |
| 18 | poly A | CTGTGCCTTCTAGTTGCCAGCCATCTGTTGTTTGCCCCTCCCCCGTGCCTTCCTTG |
| | | ACCCTGGAAGGTGCCACTCCCACTGTCCTTTCCTAATAAAATGAGGAAATTGCA |
| | | TCGCATTGTCTGAGTAGGTGTCATTCTATTCTGGGGGGTGGGGTGGGGCAGGAC |
| | | AGCAAGGGGGAGGATTGGGAAGACAATAGCAGGCATGCTGGGGA |
| 33 | H11-R | AAGTTTAGATCAGGGTGGGCAGCTCTGGGTTTTATAGGCTACACTGTTAACACT |
| | | CAGGCTGTTTTCTACCGTTTAGTCAAAATATAGTCACCTTGCCTGCTTCACCTGT |
| | | CCATCAGAGAATGGCCTCATTAATTGACTCTCTAGTATGAAGTCAAAGTAGCTT |
| | | TGGTGGCCCTAAATGGACAAGTATCAAGAGACTGGGTGAATTGAGGAGCTTGA |
| | | GACTGTCACCTCAGATCGAAAAGACTGAAAAATCACCTCAGATCAAAAAGACT |
| | | GAAAAATCTTCAGTCTGGAAAGGGGACTCAAAACCATAATTAGAGTATTCTGGT |
| | | AGAATCCTTTTCTCCACTGTTATTCATACAGTTAAGGTGAATAACTAAAAGTAAT |
| | | TGTGAGCTGAGGAGTAAGATACAACACACAAGGAATCAGTTAACAGAGTCTCG |
| | | AGTGAAATTATAAATGGAAAGAATTATGACTTGAATCATAACTCTGAGGCCCCA |
| | | TTTTCCCTAACAACTTTTGTCCCAATAAACGTGGGTATTTGTTTGGGAGAAACTA |
| | | TCATATACATGATTACCCAGTAAACAGACTGTTTACTAAGTGGGTTTAATTTTAG |
| | | AAATTGCGCGCTGC |
| 19 | ITR-R | AGGAACCCCTAGTGATGGAGTTGGCCACTCCCTCTCTGCGCGCTCGCTCGCTCA |
| | | CTGAGGCCGGGCGACCAAAGGTCGCCCGACGCCCGGGCTTTGCCCGGGCGGCC |
| | | TCAGTGAGCGAGCGAGCGCGCAGAGAGGGAGTGGCCAA |

### Example 4

The rAAV comprising the AAV capsid protein constructs is made as described above and delivered to HEK293 cells. Whole cell lysates are generated and analyzed for AAV VP capsid expression via Western Blotting.

Alternatively, non-viral expression vectors or lentiviral vectors are made using the promoter and capsid sequences, and optionally the sd/sa, 3′UTR, and/or poly A sequences, of any of the constructions shown in Table 1-6 and FIGS. 5-11 and delivered to HEK293 cells. Whole cell lysates are generated and analyzed for AAV VP capsid expression via Western Blotting.

### Example 5. In Vitro Expression Study

The rAAV comprising the liver specific promoter expressing AAV VP1 constructs were made as described above and delivered to HEK293 cells. Whole cell lysates were generated and analyzed for AAV VP capsid expression via Western Blotting.

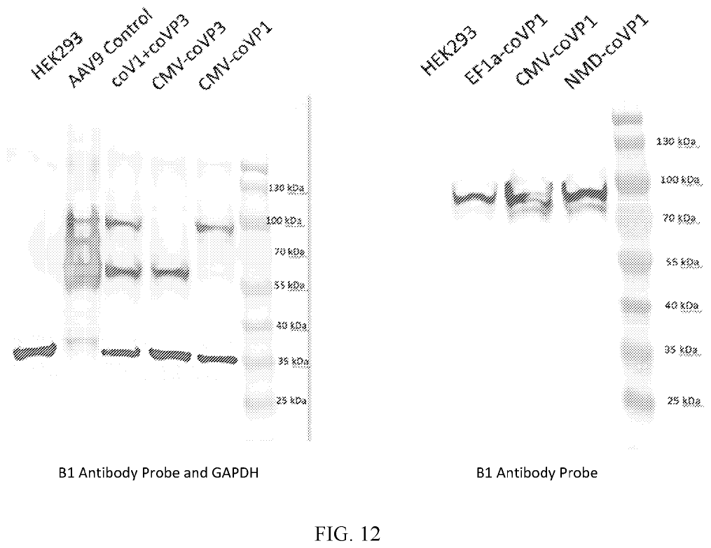

The Hek293T cells were harvested after washing with PBS in RIPA buffer with Protease inhibitors. 40 μg of protein was loaded onto 10% Tris-Glycine-SDS gel. After separation, protein was transferred onto a nitrocellulose membrane (Biorad). The western blot was then blocked with everyblot blocking buffer for 1 hr at room temperature. 1:1000 dilution of B1 antibody was diluted in blocking buffer to probed the western blot for overnight at 4° C. Next day, the western blot was washed three times in TBST at room temperature and anti-mouse secondary antibody (Vectorlabs #PI-1000-1) was used to probe the western blot. After four washes in TBST, the western blot was developed in Chemiluminescence (Millipore). The results are shown in FIG. 12.

A similar in vitro expression assay is conducted with the lenti viral vector expressing AAV VP1 and the stable GFP expressing cells (selection marker) are evaluated by Western blot assay.

### Example 6. In Vivo Expression Study

The rAAV construct comprising the AAV coVP1 (AAV9 or AAV rh74) constructs were made as described above and administered via intraperitoneal dosing to newborn C57BL/6 mice (n=6-10/group) at 1-5×10¹³ viral genomes/kg to achieve liver transduction. Two to three weeks after rAAV dosing, a second empty capsid AAV was administered to evaluate anti-AAV response.

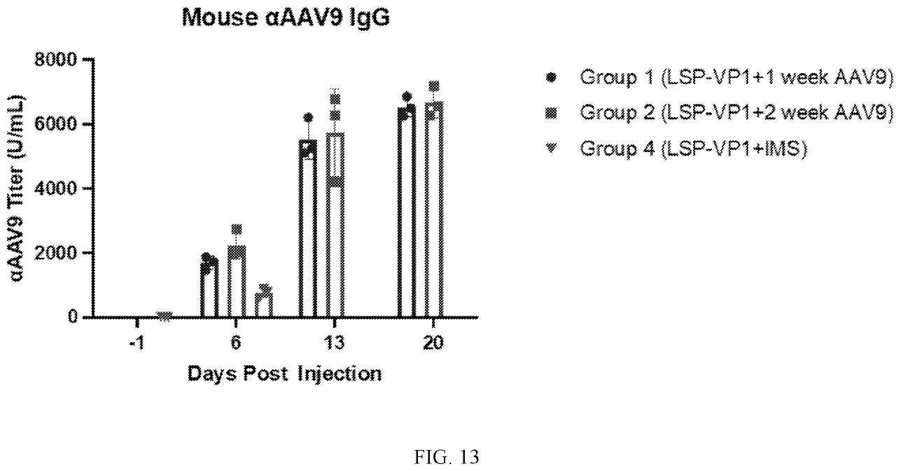

As shown in FIG. 13, when initial antibody response was blocked and LSP-VP1 was administered, there was diminished response to AAV empty particles at 6 days post challenge (group 4). In the control example without immune management pre-treatment, there was a positive response to the AAV exposure at 13 and 20 days post exposure.

Alternatively, a non-viral vector or a lentiviral vector comprising the AAV coVP1 (AAV9 or AAV rh74) constructs are made and administered, optionally via intraperitoneal dosing, to newborn C57BL/6 mice (n=6/group). Two to three weeks after rAAV dosing, a second empty capsid AAV is administered anti-AAV response is evaluated.

Alternatively, animals are challenged with AAV empty capsids or an AAV vector expressing a therapeutic or marker gene is administered. After 1-2 weeks, serum samples are analyzed for anti-AAV antibody formation.

### Example 7. In Vivo Expression Study Demonstrating Increase Expression of a Therapeutic Protein when Preventing Anti-Capsid Response

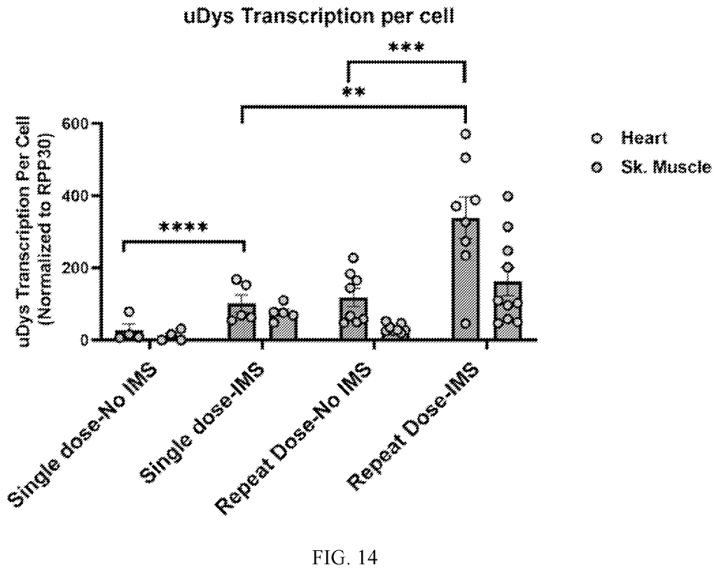

Mdx mice (DMD model) were administered an AAV vector expressing micro-dystrophin with and without immune suppression (tolerance-inducing vector dose) to prevent the anti-capsid response. Immune suppression was enabled and expression of the therapeutic gene was increase after one tolerance-inducing vector dose. In addition, multiple doses of the vector were able to be administered to achieve a greater level of expression from the therapeutic vector. In the repeat dose example, there was 4×greater expression than in the single dose example with immune suppression and over 10×greater expression when compared to single dose without immune suppression (FIG. 14).

### Example 8. Partial Bone Marrow Transplant (BMT) of Lentiviral Construct Expressing AAV VP1

Two conditions will be tested with the lentiviral vector expressing AAV VP1. First, mice will be immunized with AAV empty capsids at the dose typically used in systemic AAV administration, or about 1×10¹⁴ viral genome equivalents/kg. After 4 weeks, the anti-AAV titer will be evaluated to determine if hematopoietic stem cell (HSC) chimerism with AAV VP1 results in depletion of AAV antibodies.

The second study involves BMT prior to AAV exposure to create VP1 chimerism in HSCs. After confirmation of engraftment with VP1 positive HSCs, a challenge dose of AAV empty capsids will be administered and subsequent antibody response will be assessed.

### Example 9. Additional Sequences

TABLE 7
Sequences

| SEQ ID NO | Sequence |
|---|---|
| 1 | AAV VP3 Capsid protein |
| | MASGGGAPMADNNEGADGVGNASGNWHCDSTWLGDRVITTSTRTWALPTYNNHLYKQISSA |
| | STGASNDNHYFGYSTPWGYFDFNRFHCHFSPRDWQRLINNNWGFRPKRLNFKLFNIQVKEVTT |
| | NDGVTTIANNLTSTVQVFSDSEYQLPYVLGSAHQGCLPPFPADVFMIPQYGYLTLNNGSQAVGR |
| | SSFYCLEYFPSQMLRTGNNFTFSYTFEEVPFHSSYAHSQSLDRLMNPLIDQYLYYLNRTQNQSGS |
| | AQNKDLLFSRGSPAGMSVQPKNWLPGPCYRQQRVSKTKTDNNNSNFTWTGASKYNLNGRESII |
| | NPGTAMASHKDDEDKFFPMSGVMIFGKESAGASNTALDNVMITDEEEIKATNPVATERFGTVA |
| | VNFQSSSTDPATGDVHAMGALPGMVWQDRDVYLQGPIWAKIPHTDGHFHPSPLMGGFGLKNP |
| | PPQILIKNTPVPANPPAEFSATKFASFITQYSTGQVSVEIEWELQKENSKRWNPEVQYTSNYAKS |
| | ANVDFTVDNNGLYTEPRPIGTRYLTRPL |
| 2 | AAV VP3 capsid nucleotides sequence |
| | atggcttcaggcggtggcgcaccaatggcagacaataacgaaggcgccgacggagtgggtaatgcctcaggaaattggcattgcgattccacatggc |
| | tgggcgacagagtcatcaccaccagcacccgcacctgggccttgcccacctacaataaccacctctacaagcaaatctccagtgcttcaacgggggcc |
| | agcaacgacaaccactacttcggctacagcaccccctgggggtattttgatttcaacagattccactgccacttttcaccacgtgactggcagcgactcat |
| | caacaacaattggggattccggcccaagagactcaacttcaaactcttcaacatccaagtcaaggaggtcacgacgaatgatggcgtcacaaccatcg |
| | ctaataaccttaccagcacggttcaagtcttctcggactcggagtaccagcttccgtacgtcctcggctctgcgcaccagggctgcctccctccgttcccg |
| | gcggacgtgttcatgattccgcaatacggctacctgacgctcaacaatggcagccaagccgtgggacgttcatccttttactgcctggaatatttcccttct |
| | cagatgctgagaacgggcaacaactttaccttcagctacacctttgaggaagtgcctttccacagcagctacgcgcacagccagagcctggaccggct |
| | gatgaatcctctcatcgaccaatacctgtattacctgaacagaactcaaaatcagtccggaagtgcccaaaacaaggacttgctgtttagccgtgggtctc |
| | cagctggcatgtctgttcagcccaaaaactggctacctggaccctgttatcggcagcagcgcgtttctaaaacaaaaacagacaacaacaacagcaattt |
| | tacctggactggtgcttcaaaatataacctcaatgggcgtgaatccatcatcaaccctggcactgctatggcctcacacaaagacgacgaagacaagttc |
| | tttcccatgagcggtgtcatgatttttggaaaagagagcgccggagcttcaaacactgcattggacaatgtcatgattacagacgaagaggaaattaaag |
| | ccactaaccctgtggccaccgaaagatttgggaccgtggcagtcaatttccagagcagcagcacagaccctgcgaccggagatgtgcatgctatggg |
| | agcattacctggcatggtgtggcaagatagagacgtgtacctgcagggtcccatttgggccaaaattcctcacacagalggacactttcacccgtctcct |
| | cttatgggcggctttggactcaagaacccgcctcctcagatcctcatcaaaaacacgcctgttcctgcgaatcctccggcggagttttcagctacaaagttt |
| | gcttcattcatcacccaatactccacaggacaagtgagtgtggaaattgaatgggagctgcagaaagaaaacagcaagcgctggaatcccgaagtgca |
| | gtacacatccaattatgcaaaatctgccaacgttgattttactgtggacaacaatggactttatactgagcctcgccccattggcacccgttaccttacccgt |
| | cccctgtaa |
| 4 | pTR-LSP-AAV1-VP3 (AAV vector) |
| | ctgcaggggggggggggggggggttggccactccctctctgcgcgctcgctcgctcactgaggccgggcgaccaaaggtcgcccgacgcccggg |
| | ctttgcccgggcggcctcagtgagcgagcgagcgcgcagagagggagtggccaactccatcactaggggttcctagatctccctaaaatgggcaaac |
| | attgcaagcagcaaacagcaaacacacagccctccctgcctgctgaccttggagctggggcagaggtcagagacctctctggggactgtcccaggtc |
| | agtggtggtgcctgaagctgaggagacagggccctgtcctcgtccgtatttaagcagtggatccagaggggcaacgggggaggctgctggtgaatatt |
| | aaccaaggtcaccccagttatcggaggagcaaacaggggctaagtccactggctgggatctgagtcgcccgcctacgctgcccggacgctttgcctg |
| | ggcagtgtacagcttccactgcacttaccgaaaggagtcattgtagggccctgtctcctcagcttcaggcaccaccactgacctgggacagtgaatccg |
| | gaaagtatcaaggttacaagacaggtttaaggagaccaatagaaactgggcttgtcgagacagagaagactcttgcgtttctgataggcacctattggtc |
| | ttactgacatccactttgcctttctctccacagacgcgtggtaccgtcgacccgccaccatggcttcaggcggtggcgcaccaatggcagacaataacga |
| | aggcgccgacggagtgggtaatgcctcaggaaattggcattgcgattccacatggctgggcgacagagtcatcaccaccagcacccgcacctgggc |
| | cttgcccacctacaataaccacctctacaagcaaatctccagtgcttcaacgggggccagcaacgacaaccactacttcggctacagcaccccctggg |
| | ggtattttgatttcaacagattccactgccacttttcaccacgtgactggcagcgactcatcaacaacaattggggattccggcccaagagactcaacttca |
| | aactcttcaacatccaagtcaaggaggtcacgacgaatgatggcgtcacaaccatcgctaataaccttaccagcacggttcaagtcttctcggactcgga |
| | gtaccagcttccgtacgtcctcggctctgcgcaccagggctgcctccctccgttcccggcggacgtgttcatgattccgcaatacggctacctgacgctc |
| | aacaatggcagccaagccgtgggacgttcatccttttactgcctggaatatttcccttctcagatgctgagaacgggcaacaactttaccttcagctacacc |
| | tttgaggaagtgcctttccacagcagctacgcgcacagccagagcctggaccggctgatgaatcctctcatcgaccaatacctgtattacctgaacagaa |
| | ctcaaaatcagtccggaagtgcccaaaacaaggacttgctgtttagccgtgggtctccagctggcatgtctgttcagcccaaaaactggctacctggacc |
| | ctgttatcggcagcagcgcgtttctaaaacaaaaacagacaacaacaacagcaattttacctggactggtgcttcaaaatataacctcaatgggcgtgaat |
| | ccatcatcaaccctggcactgctatggcctcacacaaagacgacgaagacaagttctttcccatgagcggtgtcatgatttttggaaaagagagcgccg |
| | gagcttcaaacactgcattggacaatgtcatgattacagacgaagaggaaattaaagccactaaccctgtggccaccgaaagatttgggaccgtggca |
| | gtcaatttccagagcagcagcacagaccctgcgaccggagatgtgcatgctatgggagcattacctggcatggtgtggcaagatagagacgtgtacct |
| | gcagggtcccatttgggccaaaattcctcacacagatggacactttcacccgtctcctcttatgggcggctttggactcaagaacccgcctcctcagatcc |
| | tcatcaaaaacacgcctgttcctgcgaatcctccggcggagttttcagctacaaagtttgcttcattcatcacccaatactccacaggacaagtgagtgtgg |
| | aaattgaatgggagctgcagaaagaaaacagcaagcgctggaatcccgaagtgcagtacacatccaattatgcaaaatctgccaacgttgattttactgt |
| | ggacaacaatggactttatactgagcctcgccccattggcacccgttaccttacccgtcccctgtaatctagagatatcgcggccgcttcgagcagacat |
| | gataagatacattgatgagtttggacaaaccacaactagaatgcagtgaaaaaaatgctttatttgtgaaatttgtgatgctattgctttatttgtaaccattata |
| | agctgcaataaacaagttaacaacaacaattgcattcattttatgtttcaggttcagggggagatgtgggaggttttttaaagcaagtaaaacctctacaaat |
| | gtggtaaaatcgataaggatctaggaacccctagtgatggagttggccactccctctctgcgcgctcgctcgctcactgaggccgcccgggcaaagcc |
| | cgggcgtcgggcgacctttggtcgcccggcctcagtgagcgagcgagcgcgcagagagggagtggccaacccccccccccccccccctgcagcc |
| | tggcgtaatagcgaagaggcccgcaccgatcgcccttcccaacagttgcgtagcctgaatggcgaatggcgcgacgcgccctgtagcggcgcattaa |
| | gcgcggcgggtgtggtggttacgcgcagcgtgaccgctacacttgccagcgccctagcgcccgctcctttcgctttcttcccttcctttctcgccacgttc |
| | gccggctttccccgtcaagctctaaatcgggggctccctttagggttccgatttagtgctttacggcacctcgaccccaaaaaacttgattagggtgatggt |
| | tcacgtagtgggccatcgccctgatagacggtttttcgccctttgacgttggagtccacgttctttaatagtggactcttgttccaaactggaacaacactca |
| | accctatctcggtctattcttttgatttataagggattttgccgatttcggcctattggttaaaaaatgagctgatttaacaaaaatttaacgcgaattttaacaaa |
| | atattaacgtttacaatttcctgatgcggtattttctccttacgcatctgtgcggtatttcacaccgcatatggtgcactctcagtacaatctgctctgatgccgc |
| | atagttaagccagccccgacacccgccaacacccgctgacgcgccctgacgggcttgtctgctcccggcatccgcttacagacaagctgtgaccgtct |
| | ccgggagctgcatgtgtcagaggttttcaccgtcatcaccgaaacgcgcgagacgaaagggcctcgtgatacgcctatttttataggttaatgtcatgata |
| | ataatggtttcttagacgtcaggtggcacttttcggggaaatgtgcgcggaacccctatttgtttatttttctaaatacattcaaatatgtatccgctcatgagac |
| | aataaccctgataaatgcttcaataatattgaaaaaggaagagtatgagtattcaacatttccgtgtcgcccttattcccttttttgcggcattttgccttcctgtt |
| | tttgctcacccagaaacgctggtgaaagtaaaagatgctgaagatcagttgggtgcacgagtgggttacatcgaactggatctcaacagcggtaagatc |
| | cttgagagttttcgccccgaagaacgttttccaatgatgagcacttttaaagttctgctatgtggcgcggtattatcccgtattgacgccgggcaagagcaa |
| | ctcggtcgccgcatacactattctcagaatgacttggttgagtactcaccagtcacagaaaagcatcttacggatggcatgacagtaagagaattatgcag |
| | tgctgccataaccatgagtgataacactgcggccaacttacttctgacaacgatcggaggaccgaaggagctaaccgcttttttgcacaacatgggggat |
| | catgtaactcgccttgatcgttgggaaccggagctgaatgaagccataccaaacgacgagcgtgacaccacgatgcctgtagcaatggcaacaacgtt |
| | gcgcaaactattaactggcgaactacttactctagcttcccggcaacaattaatagactggatggaggggataaagttgcaggaccacttctgcgctcg |
| | gcccttccggctggctggtttattgctgataaatctggagccggtgagcgtgggtctcgcggtatcattgcagcactggggccagatggtaagccctccc |
| | gtatcgtagttatctacacgacggggagtcaggcaactatggatgaacgaaatagacagatcgctgagataggtgcctcactgattaagcattggtaact |
| | gtcagaccaagtttactcatatatactttagattgatttaaaacttcatttttaatttaaaaggatctaggtgaagatcctttttgataatctcatgaccaaaatccc |
| | ttaacgtgagttttcgttccactgagcgtcagaccccgtagaaaagatcaaaggatcttcttgagatcctttttttctgcgcgtaatctgctgcttgcaaacaa |
| | aaaaaccaccgctaccagcggtggtttgtttgccggatcaagagctaccaactctttttccgaaggtaactggcttcagcagagcgcagataccaaatac |
| | tgtccttctagtgtagccgtagttaggccaccacttcaagaactctgtagcaccgcctacatacctcgctctgctaatcctgttaccagtggctgctgccagt |
| | ggcgataagtcgtgtcttaccgggttggactcaagacgatagttaccggataaggcgcagcggtcgggctgaacggggggttcgtgcacacagccca |
| | gcttggagcgaacgacctacaccgaactgagatacctacagcgtgagcattgagaaagcgccacgcttcccgaagggagaaaggcggacaggtatc |
| | cggtaagcggcagggtcggaacaggagagcgcacgagggagcttccagggggaaacgcctggtatctttatagtcctgtcgggtttcgccacctctg |
| | acttgagcgtcgatttttgtgatgctcgtcaggggggcggagcctatggaaaaacgccagcaacgcggcctttttacggttcctggccttttgctggccttt |
| | tgctcacatgttctttcctgcgttatcccctgattctgtggataaccgtattaccgcctttgagtgagctgataccgctcgccgcagccgaacgaccgagcg |
| | cagcgagtcagtgagcgaggaagcggaagagcgcccaatacgcaaaccgcctctccccgcgcgttggccgattcattaatgcaggg |
| 5 | SARS-CoV-2 spike protein nucleic acid sequence |
| | agcttgaattcgccaccatgttcgtgtttctggtgctgctgcctctggtgtccagccagtgtgtgaacctgaccaccagaacacagctgcctccagcctac |
| | accaacagctttaccagaggcgtgtactaccccgacaaggtgttcagatccagcgtgctgcactctacccaggacctgttcctgcctttcttcagcaacgt |
| | gacctggttccacgccatccacgtgtccggcaccaatggcaccaagagattcgacaaccccgtgctgcccttcaacgacggggtgtactttgccagca |
| | ccgagaagtccaacatcatcagaggctggatcttcggcaccacactggacagcaagacccagagcctgctgatcgtgaacaacgccaccaacgtggt |
| | catcaaagtgtgcgagttccagttctgcaacgaccccttcctgggcgtctactaccacaagaacaacaagagctggatggaaagcgagttccgggtgta |
| | cagcagcgccaacaactgcaccttcgagtacgtgtcccagcctttcctgatggacctggaaggcaagcagggcaacttcaagaacctgcgcgagttcg |
| | tgttcaagaacatcgacggctacttcaagatctacagcaagcacacccctatcaacctcgtgcgggatctgcctcagggcttctctgctctggaacccctg |
| | gtggatctgcccatcggcatcaacatcacccggtttcagacactgctggccctgcacagaagctacctgacacctggcgatagcagcagcggatggac |
| | agctggtgccgccgcttactatgtgggctacctgcagcctagaaccttcctgctgaagtacaacgagaacggcaccatcaccgacgccgtggattgtgc |
| | tctggatcctctgagcgagacaaagtgcaccctgaagtccttcaccgtggaaaagggcatctaccagaccagcaacttccgggtgcagcccaccgaat |
| | ccatcgtgcggttccccaatatcaccaatctgtgccccttcggcgaggtgttcaatgccaccagattcgcctctgtgtacgcctggaaccggaagcggat |
| | cagcaattgcgtggccgactactccgtgctgtacaactccgccagcttcagcaccttcaagtgctacggcgtgtcccctaccaagctgaacgacctgtgc |
| | ttcacaaacgtgtacgccgacagcttcgtgatccggggagatgaagtgcggcagattgcccctggacagacaggcaagatcgccgactacaactaca |
| | agctgcccgacgacttcaccggctgtgtgattgcctggaacagcaacaacctggactccaaagtcggcggcaactacaattacctgtaccggctgttcc |
| | ggaagtccaatctgaagcccttcgagcgggacatctccaccgagatctatcaggccggcagcaccccttgtaacggcgtggaaggcttcaactgctac |
| | ttcccactgcagtcctacggctttcagcccacaaatggcgtgggctatcagccctacagagtggtggtgctgagcttcgaactgctgcatgcccctgcca |
| | cagtgtgcggccctaagaaaagcaccaatctcgtgaagaacaaatgcgtgaacttcaacttcaacggcctgaccggcaccggcgtgctgacagagag |
| | caacaagaagttcctgccattccagcagtttggccgggatatcgccgataccacagacgccgttagagatccccagacactggaaatcctggacatcac |
| | cccttgcagcttcggcggagtgtctgtgatcacccctggcaccaacaccagcaatcaggtggcagtgctgtaccaggacgtgaactgtaccgaagtgc |
| | ccgtggccattcacgccgatcagctgacacctacatgggggtgtactccaccggcagcaatgtgtttcagaccagagccggctgtctgatcggagcc |
| | gagcacgtgaacaatagctacgagtgcgacatccccatcggcgctggcatctgtgccagctaccagacacagacaaacagccccagacgggccaga |
| | tctgtggccagccagagcatcattgcctacacaatgtctctgggcgccgagaacagcgtggcctactccaacaactctatcgctatccccaccaacttca |
| | ccatcagcgtgaccacagagatcctgcctgtgtccatgaccaagaccagcgtggactgcaccatgtacatctgcggcgattccaccgagtgctccaac |
| | ctgctgctgcagtacggcagcttctgcacccagctgaatagagccctgacagggatcgccgtggaacaggacaagaacacccaagaggtgttcgccc |
| | aagtgaagcagatctacaagacccctcctatcaaggacttcggcggcttcaatttcagccagattctgcccgatcctagcaagcccagcaagcggagct |
| | tcatcgaggacctgctgttcaacaaagtgacactggccgacgccggcttcatcaagcagtatggcgattgtctgggcgacattgccgccagggatctga |
| | tttgcgcccagaagtttaacggactgacagtgctgcctcctctgctgaccgatgagatgatcgcccagtacacatctgccctgctggccggcacaatcac |
| | aagcggctggacatttggagctggcgccgctctgcagatcccctttgctatgcagatggcctaccggttcaacggcatcggagtgacccagaatgtgct |
| | gtacgagaaccagaagctgatcgccaaccagttcaacagcgccatcggcaagatccaggacagcctgagcagcacagcaagcgccctgggaaagc |
| | tgcaggacgtggtcaaccagaatgcccaggcactgaacaccctggtcaagcagctgtcctccaacttcggcgccatcagctctgtgctgaacgatatcc |
| | tgagcagactggaccctcctgaggccgaggtgcagatcgacagactgatcacaggcagactgcagagcctccagacatacgtgacccagcagctga |
| | tcagagccgccgagattagagcctctgccaatctggccgccaccaagatgtctgagtgtgtgctgggccagagcaagagagtggacttttgcggcaag |
| | ggctaccacctgatgagcttccctcagtctgcccctcacggcgtggtgtttctgcacgtgacatatgtgcccgctcaagagaagaatttcaccaccgctcc |
| | agccatctgccacgacggcaaagcccactttcctagagaaggcgtgttcgtgtccaacggcacccattggttcgtgacacagcggaacttctacgagc |
| | cccagatcatcaccaccgacaacaccttcgtgtctggcaactgcgacgtcgtgatcggcattgtgaacaataccgtgtacgaccctctgcagcccgagc |
| | tggacagcttcaaagaggaactggacaagtactttaagaaccacacaagccccgacgtggacctgggcgatatcagcggaatcaatgccagcgtcgt |
| | gaacatccagaaagagatcgaccggctgaacgaggtggccaagaatctgaacgagagcctgatcgacctgcaagaactggggaagtacgagcagt |
| | acatcaagtggccctggtacatctggctgggctttatcgccggactgattgccatcgtgatggtcacaatcatgctgtgttgcatgaccagctgctgtagct |
| | gcctgaagggctgttgtagctgtggcagctgctgcaagttcgacgaggacgattctgagcccgtgctgaagggcgtgaaactgcactacacctgagc |
| 6 | pTR-SARS2S-2P (Therapeutic AAV sequence) |
| | gggggggggggggggggttggccactccctctctgcgcgctcgctcgctcactgaggccgggcgaccaaaggtcgcccgacgcccgggctttgcc |
| | cgggcggcctcagtgagcgagcgagcgcgcagagagggagtggccaactccatcactaggggttcctagatcttcaatattggccattagccatattat |
| | tcattggttatatagcataaatcaatattggctattggccattgcatacgttgtatctatatcataatatgtacatttatattggctcatgtccaatatgaccgccat |
| | gttggcattgattattgactagttattaatagtaatcaattacggggtcattagttcatagcccatatatggagttccgcgttacataacttacggtaaatggcc |
| | cgcctggctgaccgcccaacgacccccgcccattgacgtcaataatgacgtatgttcccatagtaacgccaatagggactttccattgacgtcaatgggt |
| | ggagtatttacggtaaactgcccacttggcagtacatcaagtgtatcatatgccaagtccgccccctattgacgtcaatgacggtaaatggcccgcctgg |
| | cattatgcccagtacatgaccttacgggactttcctacttggcagtacatctacgtattagtcatcgctattaccatggtgatgcggttttggcagtacaccaa |
| | tgggcgtggatagcggtttgactcacggggatttccaagtctccaccccattgacgtcaatgggagtttgttttggcaccaaaatcaacgggactttccaa |
| | aatgtcgtaataaccccgccccgttgacgcaaatgggcggtaggcgtgtacggtgggaggtctatataagcagagctcgtttagtgaaccgtcagatca |
| | ctagaagcttgaattcgccaccatgttcgtgtttctggtgctgctgcctctggtgtccagccagtgtgtgaacctgaccaccagaacacagctgcctccag |
| | cctacaccaacagctttaccagaggcgtgtactaccccgacaaggtgttcagatccagcgtgctgcactctacccaggacctgttcctgcctttcttcagc |
| | aacgtgacctggttccacgccatccacgtgtccggcaccaatggcaccaagagattcgacaaccccgtgctgcccttcaacgacggggtgtactttgcc |
| | agcaccgagaagtccaacatcatcagaggctggatcttcggcaccacactggacagcaagacccagagcctgctgatcgtgaacaacgccaccaac |
| | gtggtcatcaaagtgtgcgagttccagttctgcaacgaccccttcctgggcgtctactaccacaagaacaacaagagctggatggaaagcgagttccgg |
| | gtgtacagcagcgccaacaactgcaccttcgagtacgtgtcccagcctttcctgatggacctggaaggcaagcagggcaacttcaagaacctgcgcga |
| | gttcgtgttcaagaacatcgacggctacttcaagatctacagcaagcacacccctatcaacctcgtgcgggatctgcctcagggcttctctgctctggaac |
| | ccctggtggatctgcccatcggcatcaacatcacccggtttcagacactgctggccctgcacagaagctacctgacacctggcgatagcagcagcgga |
| | tggacagctggtgccgccgcttactatgtgggctacctgcagcctagaaccttcctgctgaagtacaacgagaacggcaccatcaccgacgccgtgga |
| | ttgtgctctggatcctctgagcgagacaaagtgcaccctgaagtccttcaccgtggaaaagggcatctaccagaccagcaacttccgggtgcagccca |
| | ccgaatccatcgtgcggttccccaatatcaccaatctgtgccccttcggcgaggtgttcaatgccaccagattcgcctctgtgtacgcctggaaccggaa |
| | gcggatcagcaattgcgtggccgactactccgtgctgtacaactccgccagcttcagcaccttcaagtgctacggcgtgtcccctaccaagctgaacga |
| | cctgtgcttcacaaacgtgtacgccgacagcttcgtgatccggggagatgaagtgcggcagattgcccctggacagacaggcaagatcgccgactac |
| | aactacaagctgcccgacgacttcaccggctgtgtgattgcctggaacagcaacaacctggactccaaagtcggcggcaactacaattacctgtaccg |
| | gctgttccggaagtccaatctgaagcccttcgagcgggacatctccaccgagatctatcaggccggcagcaccccttgtaacggcgtggaaggcttca |
| | actgctacttcccactgcagtcctacggctttcagcccacaaatggcgtgggctatcagccctacagagtggtggtgctgagcttcgaactgctgcatgc |
| | ccctgccacagtgtgcggccctaagaaaagcaccaatctcgtgaagaacaaatgcgtgaacttcaacttcaacggcctgaccggcaccggcgtgctga |
| | cagagagcaacaagaagttcctgccattccagcagtttggccgggatatcgccgataccacagacgccgttagagatccccagacactggaaatcctg |
| | gacatcaccccttgcagcttcggcggagtgtctgtgatcacccctggcaccaacaccagcaatcaggtggcagtgctgtaccaggacgtgaactgtac |
| | cgaagtgcccgtggccattcacgccgatcagctgacacctacatggcgggtgtactccaccggcagcaatgtgtttcagaccagagccggctgtctga |
| | tcggagccgagcacgtgaacaatagctacgagtgcgacatccccatcggcgctggcatctgtgccagctaccagacacagacaaacagccccagac |
| | gggccagatctgtggccagccagagcatcattgcctacacaatgtctctgggcgccgagaacagcgtggcctactccaacaactctatcgctatcccca |
| | ccaacttcaccatcagcgtgaccacagagatcctgcctgtgtccatgaccaagaccagcgtggactgcaccatgtacatctgcggcgattccaccgagt |
| | gctccaacctgctgctgcagtacggcagcttctgcacccagctgaatagagccctgacagggatcgccgtggaacaggacaagaacacccaagagg |
| | tgttcgcccaagtgaagcagatctacaagacccctcctatcaaggacttcggcggcttcaatttcagccagattctgcccgatcctagcaagcccagcaa |
| | gcggagcttcatcgaggacctgctgttcaacaaagtgacactggccgacgccggcttcatcaagcagtatggcgattgtctgggcgacattgccgcca |
| | gggatctgatttgcgcccagaagtttaacggactgacagtgctgcctcctctgctgaccgatgagatgatcgcccagtacacatctgccctgctggccgg |
| | cacaatcacaagcggctggacatttggagctggcgccgctctgcagatcccctttgctatgcagatggcctaccggttcaacggcatcggagtgaccca |
| | gaatgtgctgtacgagaaccagaagctgatcgccaaccagttcaacagcgccatcggcaagatccaggacagcctgagcagcacagcaagcgccct |
| | gggaaagctgcaggacgtggtcaaccagaatgcccaggcactgaacaccctggtcaagcagctgtcctccaacttcggcgccatcagctctgtgctg |
| | aacgatatcctgagcagactggaccctcctgaggccgaggtgcagatcgacagactgatcacaggcagactgcagagcctccagacatacgtgaccc |
| | agcagctgatcagagccgccgagattagagcctctgccaatctggccgccaccaagatgtctgagtgtgtgctgggccagagcaagagagtggacttt |
| | tgcggcaagggctaccacctgatgagcttccctcagtctgcccctcacggcgtggtgtttctgcacgtgacatatgtgcccgctcaagagaagaatttca |
| | ccaccgctccagccatctgccacgacggcaaagcccactttcctagagaaggcgtgttcgtgtccaacggcacccattggttcgtgacacagcggaac |
| | ttctacgagccccagatcatcaccaccgacaacaccttcgtgtctggcaactgcgacgtcgtgatcggcattgtgaacaataccgtgtacgaccctctgc |
| | agcccgagctggacagcttcaaagaggaactggacaagtactttaagaaccacacaagccccgacgtggacctgggcgatatcagcggaatcaatgc |
| | cagcgtcgtgaacatccagaaagagatcgaccggctgaacgaggtggccaagaatctgaacgagagcctgatcgacctgcaagaactggggaagta |
| | cgagcagtacatcaagtggccctggtacatctggctgggctttatcgccggactgattgccatcgtgatggtcacaatcatgctgtgttgcatgaccagct |
| | gctgtagctgcctgaagggctgttgtagctgtggcagctgctgcaagttcgacgaggacgattctgagcccgtgctgaagggcgtgaaactgcactac |
| | acctgagcggccgcttcgagcagacatgataagatacattgatgagtttggacaaaccacaactagaatgcagtgaaaaaaatgctttatttgtgaaattt |
| | gtgatgctattgctttatttgtaaccattataagctgcaataaacaagttaacaacaacaattgcattcattttatgtttcaggttcagggggagatgtgggagg |
| | ttttttaaagcaagtaaaacctctacaaatgtggtaaaatcgataaggatctaggaacccctagtgatggagttggccactccctctctgcgcgctcgctcg |
| | ctcactgaggccgcccgggcaaagcccgggcgtcgggcgacctttggtcgcccggcctcagtgagcgagcgagcgcgcagagagggagtggcc |
| | aacccccccccccccccccctgcagcctggcgtaatagcgaagaggcccgcaccgatcgcccttcccaacagttgcgtagcctgaatggcgaatggc |
| | gcgacgcgccctgtagcggcgcattaagcgcggcgggtgtggtggttacgcgcagcgtgaccgctacacttgccagcgccctagcgcccgctccttt |
| | cgctttcttcccttcctttctcgccacgttcgccggctttccccgtcaagctctaaatcgggggctccctttagggttccgatttagtgctttacggcacctcg |
| | accccaaaaaacttgattagggtgatggttcacgtagtgggccatcgccctgatagacggtttttcgccctttgacgttggagtccacgttctttaatagtgg |
| | actcttgttccaaactggaacaacactcaaccctatctcggtctattcttttgatttataagggattttgccgatttcggcctattggttaaaaaatgagctgattt |
| | aacaaaaatttaacgcgaattttaacaaaatattaacgtttacaatttcctgatgcggtattttctccttacgcatctgtgcggtatttcacaccgcatatggtgc |
| | actctcagtacaatctgctctgatgccgcatagttaagccagccccgacacccgccaacacccgctgacgcgccctgacgggcttgtctgctcccggca |
| | tccgcttacagacaagctgtgaccgtctccgggagctgcatgtgtcagaggttttcaccgtcatcaccgaaacgcgcgagacgaaagggcctcgtgat |
| | acgcctatttttataggttaatgtcatgataataatggtttcttagacgtcaggtggcacttttcggggaaatgtgcgcggaacccctatttgtttatttttctaaa |
| | tacattcaaatatgtatccgctcatgagacaataaccctgataaatgcttcaataatattgaaaaaggaagagtatgagtattcaacatttccgtgtcgccctt |
| | attcccttttttgcggcattttgccttcctgtttttgctcacccagaaacgctggtgaaagtaaaagatgctgaagatcagttgggtgcacgagtgggttacat |
| | cgaactggatctcaacagcggtaagatccttgagagttttcgccccgaagaacgttttccaatgatgagcacttttaaagttctgctatgtggcgcggtatta |
| | tcccgtattgacgccgggcaagagcaactcggtcgccgcatacactattctcagaatgacttggttgagtactcaccagtcacagaaaagcatcttacgg |
| | atggcatgacagtaagagaattatgcagtgctgccataaccatgagtgataacactgcggccaacttacttctgacaacgatcggaggaccgaaggag |
| | ctaaccgcttttttgcacaacatgggggatcatgtaactcgccttgatcgttgggaaccggagctgaatgaagccataccaaacgacgagcgtgacacc |
| | acgatgcctgtagcaatggcaacaacgttgcgcaaactattaactggcgaactacttactctagcttcccggcaacaattaatagactggatggaggcgg |
| | ataaagttgcaggaccacttctgcgctcggcccttccggctggctggtttattgctgataaatctggagccggtgagcgtgggtctcgcggtatcattgca |
| | gcactggggccagatggtaagccctcccgtatcgtagttatctacacgacggggagtcaggcaactatggatgaacgaaatagacagatcgctgagat |
| | aggtgcctcactgattaagcattggtaactgtcagaccaagtttactcatatatactttagattgatttaaaacttcatttttaatttaaaaggatctaggtgaag |
| | atcctttttgataatctcatgaccaaaatcccttaacgtgagttttcgttccactgagcgtcagaccccgtagaaaagatcaaaggatcttcttgagatcctttt |
| | tttctgcgcgtaatctgctgcttgcaaacaaaaaaaccaccgctaccagcggtggtttgtttgccggatcaagagctaccaactctttttccgaaggtaact |
| | ggcttcagcagagcgcagataccaaatactgtccttctagtgtagccgtagttaggccaccacttcaagaactctgtagcaccgcctacatacctcgctct |
| | gctaatcctgttaccagtggctgctgccagtggcgataagtcgtgtcttaccgggttggactcaagacgatagttaccggataaggcgcagcggtcggg |
| | ctgaacggggggttcgtgcacacagcccagcttggagcgaacgacctacaccgaactgagatacctacagcgtgagcattgagaaagcgccacgctt |
| | cccgaagggagaaaggcggacaggtatccggtaagcggcagggtcggaacaggagagcgcacgagggagcttccagggggaaacgcctggtat |
| | ctttalagtcctgtcgggtttcgccacctctgacttgagcgtcgatttttgtgatgctcgtcaggggggggagcctatggaaaaacgccagcaacgcgg |
| | cctttttacggttcctggccttttgctggccttttgctcacatgttctttcctgcgttatcccctgattctgtggataaccgtattaccgcctttgagtgagctgata |
| | ccgctcgccgcagccgaacgaccgagcgcagcgagtcagtgagcgaggaagcggaagagcgcccaatacgcaaaccgcctctccccgcgcgttg |
| | gccgattcattaatgcagggctgcag |
| 7 | SARS-CoV-2 Spike Protein deleted TM domain |
| | agcttgaattcgccaccatgttcgtgtttctggtgctgctgcctctggtgtccagccagtgtgtgaacctgaccaccagaacacagctgcctccagcctac |
| | accaacagctttaccagaggcgtgtactaccccgacaaggtgttcagatccagcgtgctgcactctacccaggacctgttcctgcctttcttcagcaacgt |
| | gacctggttccacgccatccacgtgtccggcaccaatggcaccaagagattcgacaaccccgtgctgcccttcaacgacggggtgtactttgccagca |
| | ccgagaagtccaacatcatcagaggctggatcttcggcaccacactggacagcaagacccagagcctgctgatcgtgaacaacgccaccaacgtggt |
| | catcaaagtgtgcgagttccagttctgcaacgaccccttcctgggcgtctactaccacaagaacaacaagagctggatggaaagcgagttccgggtgta |
| | cagcagcgccaacaactgcaccttcgagtacgtgtcccagcctttcctgatggacctggaaggcaagcagggcaacttcaagaacctgcgcgagttcg |
| | tgttcaagaacatcgacggctacttcaagatctacagcaagcacacccctatcaacctcgtgcgggatctgcctcagggcttctctgctctggaacccctg |
| | gtggatctgcccatcggcatcaacatcacccggtttcagacactgctggccctgcacagaagctacctgacacctggcgatagcagcagcggatggac |
| | agctggtgccgccgcttactatgtgggctacctgcagcctagaaccttcctgctgaagtacaacgagaacggcaccatcaccgacgccgtggattgtgc |
| | tctggatcctctgagcgagacaaagtgcaccctgaagtccttcaccgtggaaaagggcatctaccagaccagcaacttccgggtgcagcccaccgaat |
| | ccatcgtgggttccccaatatcaccaatctgtgccccttcggcgaggtgttcaatgccaccagattcgcctctgtgtacgcctggaaccggaagcggat |
| | cagcaattgcgtggccgactactccgtgctgtacaactccgccagcttcagcaccttcaagtgctacggcgtgtcccctaccaagctgaacgacctgtgc |
| | ttcacaaacgtgtacgccgacagcttcgtgatccggggagatgaagtgcggcagattgcccctggacagacaggcaagatcgccgactacaactaca |
| | agctgcccgacgacttcaccggctgtgtgattgcctggaacagcaacaacctggactccaaagtcggcggcaactacaattacctgtaccggctgttcc |
| | ggaagtccaatctgaagcccttcgagcgggacatctccaccgagatctatcaggccggcagcaccccttgtaacggcgtggaaggcttcaactgctac |
| | ttcccactgcagtcctacggctttcagcccacaaatggcgtgggctatcagccctacagagtggtggtgctgagcttcgaactgctgcatgcccctgcca |
| | cagtgtgcggccctaagaaaagcaccaatctcgtgaagaacaaatgcgtgaacttcaacttcaacggcctgaccggcaccggcgtgctgacagagag |
| | caacaagaagttcctgccattccagcagtttggccgggatatcgccgataccacagacgccgttagagatccccagacactggaaatcctggacatcac |
| | cccttgcagcttcggcggagtgtctgtgatcacccctggcaccaacaccagcaatcaggtggcagtgctgtaccaggacgtgaactgtaccgaagtgc |
| | ccgtggccattcacgccgatcagctgacacctacatggcgggtgtactccaccggcagcaatgtgtttcagaccagagccggctgtctgatcggagcc |
| | gagcacgtgaacaatagctacgagtgcgacatccccatcggcgctggcatctgtgccagctaccagacacagacaaacagccccagacgggccaga |
| | tctgtggccagccagagcatcattgcctacacaatgtctctgggcgccgagaacagcgtggcctactccaacaactctatcgctatccccaccaacttca |
| | ccatcagcgtgaccacagagatcctgcctgtgtccatgaccaagaccagcgtggactgcaccatgtacatctgcggcgattccaccgagtgctccaac |
| | ctgctgctgcagtacggcagcttctgcacccagctgaatagagccctgacagggatcgccgtggaacaggacaagaacacccaagaggtgttcgccc |
| | aagtgaagcagatctacaagacccctcctatcaaggacttcggcggcttcaatttcagccagattctgcccgatcctagcaagcccagcaagcggagct |
| | tcatcgaggacctgctgttcaacaaagtgacactggccgacgccggcttcatcaagcagtatggcgattgtctgggcgacattgccgccagggatctga |
| | tttgcgcccagaagtttaacggactgacagtgctgcctcctctgctgaccgatgagatgatcgcccagtacacatctgccctgctggccggcacaatcac |
| | aagcggctggacatttggagctggcgccgctctgcagatcccctttgctatgcagatggcctaccggttcaacggcatcggagtgacccagaatgtgct |
| | gtacgagaaccagaagctgatcgccaaccagttcaacagcgccatcggcaagatccaggacagcctgagcagcacagcaagcgccctgggaaagc |
| | tgcaggacgtggtcaaccagaatgcccaggcactgaacaccctggtcaagcagctgtcctccaacttcggcgccatcagctctgtgctgaacgatatcc |
| | tgagcagactggaccctcctgaggccgaggtgcagatcgacagactgatcacaggcagactgcagagcctccagacatacgtgacccagcagctga |
| | tcagagccgccgagattagagcctctgccaatctggccgccaccaagatgtctgagtgtgtgctgggccagagcaagagagtggacttttgcggcaag |
| | ggctaccacctgatgagcttccctcagtctgcccctcacggcgtggtgtttctgcacgtgacatatgtgcccgctcaagagaagaatttcaccaccgctcc |
| | agccatctgccacgacggcaaagcccactttcctagagaaggcgtgttcgtgtccaacggcacccattggttcgtgacacagcggaacttctacgagc |
| | cccagatcatcaccaccgacaacaccttcgtgtctggcaactgcgacgtcgtgatcggcattgtgaacaataccgtgtacgaccctctgcagcccgagc |
| | tggacagcttcaaagaggaactggacaagtactttaagaaccacacaagccccgacgtggacctgggcgatatcagcggaatcaatgccagcgtcgt |
| | gaacatccagaaagagatcgaccggctgaacgaggtggccaagaatctgaacgagagcctgatcgacctgcaagaactggggaagtacgagggct |
| | acalccctgaggctcctagagatggccaggcctacgtcagaaaggatggcgagtgggtcctgctgagcaccttcctgtaatctagagc |
| 8 | p-TR-SARS2-2P-dTM (Therapeutic AAV sequence) |
| | gggggggggggggggggttggccactccctctctgcgcgctcgctcgctcactgaggccggggaccaaaggtcgcccgacgcccgggctttgcc |
| | cgggcggcctcagtgagcgagcgagcgcgcagagagggagtggccaactccatcactaggggttcctagatcttcaatattggccattagccatattat |
| | tcattggttatatagcataaatcaatattggctattggccattgcatacgttgtatctatatcataatatgtacatttatattggctcatgtccaatatgaccgccat |
| | gttggcattgattattgactagttattaatagtaatcaattacggggtcattagttcatagcccatatatggagttccgcgttacataacttacggtaaatggcc |
| | cgcctggctgaccgcccaacgacccccgcccattgacgtcaataatgacgtatgttcccatagtaacgccaatagggactttccattgacgtcaatgggt |
| | ggagtatttacggtaaactgcccacttggcagtacatcaagtgtatcatatgccaagtccgccccctattgacgtcaatgacggtaaatggcccgcctgg |
| | cattatgcccagtacatgaccttacgggactttcctacttggcagtacatctacgtattagtcatcgctattaccatggtgatgcggttttggcagtacaccaa |
| | tgggcgtggatagcggtttgactcacggggatttccaagtctccaccccattgacgtcaatgggagtttgttttggcaccaaaatcaacgggactttccaa |
| | aatgtcgtaataaccccgccccgttgacgcaaatgggcggtaggcgtgtacggtgggaggtctatataagcagagctcgtttagtgaaccgtcagatca |
| | ctagaagcttgaattcgccaccatgttcgtgtttctggtgctgctgcctctggtgtccagccagtgtgtgaacctgaccaccagaacacagctgcctccag |
| | cctacaccaacagctttaccagaggcgtgtactaccccgacaaggtgttcagatccagcgtgctgcactctacccaggacctgttcctgcctttcttcagc |
| | aacgtgacctggttccacgccatccacgtgtccggcaccaatggcaccaagagattcgacaaccccgtgctgcccttcaacgacggggtgtactttgcc |
| | agcaccgagaagtccaacatcatcagaggctggatcttcggcaccacactggacagcaagacccagagcctgctgatcgtgaacaacgccaccaac |
| | gtggtcatcaaagtgtgcgagttccagttctgcaacgaccccttcctgggcgtctactaccacaagaacaacaagagctggatggaaagcgagttccgg |
| | gtgtacagcagcgccaacaactgcaccttcgagtacgtgtcccagcctttcctgatggacctggaaggcaagcagggcaacttcaagaacctgcgcga |
| | gttcgtgttcaagaacatcgacggctacttcaagatctacagcaagcacacccctatcaacctcgtgcgggatctgcctcagggcttctctgctctggaac |
| | ccclggtggatctgcccatcggcatcaacatcacccggtttcagacactgctggccctgcacagaagctacctgacacctggcgatagcagcagcgga |
| | tggacagctggtgccgccgcttactatgtgggctacctgcagcctagaaccttcctgctgaagtacaacgagaacggcaccatcaccgacgccgtgga |
| | ttgtgctctggatcctctgagcgagacaaagtgcaccctgaagtccttcaccgtggaaaagggcatctaccagaccagcaacttccgggtgcagccca |
| | ccgaatccatcgtgcggttccccaatatcaccaatctgtgccccttcggcgaggtgttcaatgccaccagattcgcctctgtgtacgcctggaaccggaa |
| | gcggatcagcaattgcgtggccgactactccgtgctgtacaactccgccagcttcagcaccttcaagtgctacggcgtgtcccctaccaagctgaacga |
| | cctgtgcttcacaaacgtgtacgccgacagcttcgtgatccggggagatgaagtgcggcagattgcccctggacagacaggcaagatcgccgactac |
| | aactacaagctgcccgacgacttcaccggctgtgtgattgcctggaacagcaacaacctggactccaaagtcggcggcaactacaattacctgtaccg |
| | gctgttccggaagtccaatctgaagcccttcgagcgggacatctccaccgagatctatcaggccggcagcaccccttgtaacggcgtggaaggcttca |
| | actgctacttcccactgcagtcctacggctttcagcccacaaatggcgtgggctatcagccctacagagtggtggtgctgagcttcgaactgctgcatgc |
| | ccctgccacagtgtgcggccctaagaaaagcaccaatctcgtgaagaacaaatgcgtgaacttcaacttcaacggcctgaccggcaccggcgtgctga |
| | cagagagcaacaagaagttcctgccattccagcagtttggccgggatatcgccgataccacagacgccgttagagatccccagacactggaaatcctg |
| | gacatcaccccttgcagcttcggcggagtgtctgtgatcacccctggcaccaacaccagcaatcaggtggcagtgctgtaccaggacgtgaactgtac |
| | cgaagtgcccgtggccattcacgccgatcagctgacacctacatggcgggtgtactccaccggcagcaatgtgtttcagaccagagccggctgtctga |
| | tcggagccgagcacgtgaacaatagctacgagtgcgacatccccatcggcgctggcatctgtgccagctaccagacacagacaaacagccccagac |
| | gggccagatctgtggccagccagagcatcattgcctacacaatgtctctgggcgccgagaacagcgtggcctactccaacaactctatcgctatcccca |
| | ccaacttcaccatcagcgtgaccacagagatcctgcctgtgtccatgaccaagaccagcgtggactgcaccatgtacatctgcggcgattccaccgagt |
| | gctccaacctgctgctgcagtacggcagcttctgcacccagctgaatagagccctgacagggatcgccgtggaacaggacaagaacacccaagagg |
| | tgttcgcccaagtgaagcagatctacaagacccctcctatcaaggacttcggcggcttcaatttcagccagattctgcccgatcctagcaagcccagcaa |
| | gcggagcttcatcgaggacctgctgttcaacaaagtgacactggccgacgccggcttcatcaagcagtatggcgattgtctgggcgacattgccgcca |
| | gggatctgatttgcgcccagaagtttaacggactgacagtgctgcctcctctgctgaccgatgagatgatcgcccagtacacatctgccctgctggccgg |
| | cacaatcacaagcggctggacatttggagctggcgccgctctgcagatcccctttgctatgcagatggcctaccggttcaacggcatcggagtgaccca |
| | gaatgtgctgtacgagaaccagaagctgatcgccaaccagttcaacagcgccatcggcaagatccaggacagcctgagcagcacagcaagcgccct |
| | gggaaagctgcaggacgtggtcaaccagaatgcccaggcactgaacaccctggtcaagcagctgtcctccaacttcggcgccatcagctctgtgctg |
| | aacgatatcctgagcagactggaccctcctgaggccgaggtgcagatcgacagactgatcacaggcagactgcagagcctccagacatacgtgaccc |
| | agcagctgatcagagccgccgagattagagcctctgccaatctggccgccaccaagatgtctgagtgtgtgctgggccagagcaagagagtggacttt |
| | tgcggcaagggctaccacctgatgagcttccctcagtctgcccctcacggcgtggtgtttctgcacgtgacatatgtgcccgctcaagagaagaatttca |
| | ccaccgctccagccatctgccacgacggcaaagcccactttcctagagaaggcgtgttcgtgtccaacggcacccattggttcgtgacacagcggaac |
| | ttctacgagccccagatcatcaccaccgacaacaccttcgtgtctggcaactgcgacgtcgtgatcggcattgtgaacaataccgtgtacgaccctctgc |
| | agcccgagctggacagcttcaaagaggaactggacaagtactttaagaaccacacaagccccgacgtggacctgggcgatatcagcggaatcaatgc |
| | cagcgtcgtgaacatccagaaagagatcgaccggctgaacgaggtggccaagaatctgaacgagagcctgatcgacctgcaagaactggggaagta |
| | cgagggctacatccctgaggctcctagagatggccaggcctacgtcagaaaggatggcgagtgggtcctgctgagcaccttcctgtaatctagagcgg |
| | ccgcttcgagcagacatgataagatacattgatgagtttggacaaaccacaactagaatgcagtgaaaaaaatgctttatttgtgaaatttgtgatgctattg |
| | ctttatttgtaaccattataagctgcaataaacaagttaacaacaacaattgcattcattttatgtttcaggttcagggggagatgtgggaggttttttaaagca |
| | agtaaaacctctacaaatgtggtaaaatcgataaggatctaggaacccctagtgatggagttggccactccctctctgcgcgctcgctcgctcactgagg |
| | ccgcccgggcaaagcccgggcgtcgggcgacctttggtcgcccggcctcagtgagcgagcgagcgcgcagagagggagtggccaacccccccc |
| | cccccccccctgcagcctggcgtaatagcgaagaggcccgcaccgatcgcccttcccaacagttgcgtagcctgaatggcgaatggcgcgacgcgc |
| | cctgtagcggcgcattaagcgcggcgggtgtggtggttacgcgcagcgtgaccgctacacttgccagcgccctagcgcccgctcctttcgctttcttcc |
| | cttcctttctcgccacgttcgccggctttccccgtcaagctctaaatcgggggctccctttagggttccgatttagtgctttacggcacctcgaccccaaaaa |
| | acttgattagggtgatggttcacgtagtgggccatcgccctgatagacggtttttcgccctttgacgttggagtccacgttctttaatagtggactcttgttcc |
| | aaactggaacaacactcaaccctatctcggtctattcttttgatttataagggattttgccgatttcggcctattggttaaaaaatgagctgatttaacaaaaatt |
| | taacgcgaattttaacaaaatattaacgtttacaatttcctgatgcggtattttctccttacgcatctgtgcggtatttcacaccgcatatggtgcactctcagta |
| | caatctgctctgatgccgcatagttaagccagccccgacacccgccaacacccgctgacgcgccctgacgggcttgtctgctcccggcatccgcttaca |
| | gacaagctgtgaccgtctccgggagctgcatgtgtcagaggttttcaccgtcatcaccgaaacgcgcgagacgaaagggcctcgtgatacgcctattttt |
| | ataggttaatgtcatgataataatggtttcttagacgtcaggtggcacttttcggggaaatgtgcgcggaacccctatttgtttatttttctaaatacattcaaat |
| | atgtatccgctcatgagacaataaccctgataaatgcttcaataatattgaaaaaggaagagtatgagtattcaacatttccgtgtcgcccttattccctttttt |
| | gcggcattttgccttcctgtttttgctcacccagaaacgctggtgaaagtaaaagatgctgaagatcagttgggtgcacgagtgggttacatcgaactgga |
| | tctcaacagcggtaagatccttgagagttttcgccccgaagaacgttttccaatgatgagcacttttaaagttctgctatgtggcgcggtattatcccgtattg |
| | acgccgggcaagagcaactcggtcgccgcatacactattctcagaatgacttggttgagtactcaccagtcacagaaaagcatcttacggatggcatga |
| | cagtaagagaattatgcagtgctgccataaccatgagtgataacactgcggccaacttacttctgacaacgatcggaggaccgaaggagctaaccgctt |
| | ttttgcacaacatgggggatcatgtaactcgccttgatcgttgggaaccggagctgaatgaagccataccaaacgacgagcgtgacaccacgatgcctg |
| | tagcaatggcaacaacgttgcgcaaactattaactggcgaactacttactctagcttcccggcaacaattaatagactggatggaggcggataaagttgc |
| | aggaccacttctgcgctcggcccttccggctggctggtttattgctgataaatctggagccggtgagcgtgggtctcgcggtatcattgcagcactgggg |
| | ccagatggtaagccctcccgtatcgtagttatctacacgacggggagtcaggcaactatggatgaacgaaatagacagatcgctgagataggtgcctca |
| | ctgattaagcattggtaactgtcagaccaagtttactcatatatactttagattgatttaaaacttcatttttaatttaaaaggatctaggtgaagatcctttttgata |
| | atctcatgaccaaaatcccttaacgtgagttttcgttccactgagcgtcagaccccgtagaaaagatcaaaggatcttcttgagatcctttttttctgcgcgta |
| | atctgctgcttgcaaacaaaaaaaccaccgctaccagcggtggtttgtttgccggatcaagagctaccaactctttttccgaaggtaactggcttcagcag |
| | agcgcagataccaaatactgtccttctagtgtagccgtagttaggccaccacttcaagaactctgtagcaccgcctacatacctcgctctgctaatcctgtt |
| | accagtggctgctgccagtggcgataagtcgtgtcttaccgggttggactcaagacgatagttaccggataaggcgcagcggtcgggctgaacgggg |
| | ggttcgtgcacacagcccagcttggagcgaacgacctacaccgaactgagatacctacagcgtgagcattgagaaagcgccacgcttcccgaaggg |
| | agaaaggcggacaggtatccggtaagcggcagggtcggaacaggagagcgcacgagggagcttccagggggaaacgcctggtatctttatagtcct |
| | gtcgggtttcgccacctctgacttgagcgtcgatttttgtgatgctcgtcaggggggggagcctatggaaaaacgccagcaacgcggcctttttacggtt |
| | cctggccttttgctggccttttgctcacatgttctttcctgcgttatcccctgattctgtggataaccgtattaccgcctttgagtgagctgataccgctcgccg |
| | cagccgaacgaccgagcgcagcgagtcagtgagcgaggaagcggaagagcgcccaatacgcaaaccgcctctccccgcgcgttggccgattcatt |
| | aatgcagggctgcag |
| 9 | CMV promoter |
| | tcaatattggccattagccatattattcattggttatatagcataaatcaatattggctattggccattgcatacgttgtatctatatcataatatgtacatttatatt |
| | ggctcatgtccaatatgaccgccatgttggcattgattattgactagttattaatagtaatcaattacggggtcattagttcatagcccatatatggagttccgc |
| | gttacataacttacggtaaatggcccgcctggctgaccgcccaacgacccccgcccattgacgtcaataatgacgtatgttcccatagtaacgccaatag |
| | ggactttccattgacgtcaatgggtggagtatttacggtaaactgcccacttggcagtacatcaagtgtatcatatgccaagtccgccccctattgacgtca |
| | atgacggtaaatggcccgcctggcattatgcccagtacatgaccttacgggactttcctacttggcagtacatctacgtattagtcatcgctattaccatggt |
| | gatgcggttttggcagtacaccaatgggcgtggatagcggtttgactcacggggatttccaagtctccaccccattgacgtcaatgggagtttgttttggc |
| | accaaaatcaacgggactttccaaaatgtcgtaataaccccgccccgttgacgcaaatgggcggtaggcgtgtacggtgggaggtctatataagcaga |
| | gctcgtttagtgaaccgtcagatcactaga |
| 10 | pTR2-LSP-Cap9_Dual4 plasmid (AAV plasmid) |
| | ttaattaactgcagacgccagctgttggccactccctctctgcgcgctcgctcgctcactgaggccgggcgaccaaaggtcgcccgacgcccgggcttt |
| | gcccgggcggcctcagtgagcgagcgagcgcgcagagagggagtggccaactccatcactaggggttccttctagaggcgcgccaagcttccctaa |
| | aatgggcaaacattgcaagcagcaaacagcaaacacacagccctccctgcctgctgaccttggagctggggcagaggtcagagacctctctgggga |
| | ctgtcccaggtcagtggtggtgcctgaagctgaggagacagggccctgtcctcgtccgtatttaagcagtggatccagaggggcaacgggggaggct |
| | gctggtgaatattaaccaaggtcaccccagttatcggaggagcaaacaggggctaagtccactggctgggatctgagtcgcccgcctacgctgcccg |
| | gacgctttgcctgggcagtgtacagcttccactgcacttaccgaaaggagtcattgtagggccctgtctcctcagcttcaggcaccaccactgacctggg |
| | acagtgaatccggaactagtaagtatcaaggttacaagacaggtttaaggagaccaatagaaactgggcttgtcgagacagagaagactcttgcgtttct |
| | gataggcacctattggtcttactgacatccactttgcctttctctccacaggctagcatttaaatcaggtatggctgccgatggttatcttccagattggctcg |
| | aggacaaccttagtgaaggaattcgcgagtggtgggctttgaaacctggagcccctcaacccaaggcaaatcaacaacatcaagacaacgctcgagg |
| | tcttgtgcttccgggttacaaataccttggacccggcaacggactcgacaagggggagccggtcaacgcagcagacgcggcggccctcgagcacga |
| | caaggcctacgaccagcagctcaaggccggagacaacccgtacctcaagtacaaccacgccgacgccgagttccaggagcggctcaaagaagata |
| | cgtcttttgggggcaacctcgggcgagcagtcttccaggccaaaaagaggcttcttgaacctcttggtctggttgaggaagcggctaagacggctcctg |
| | gaaagaagaggcctgtagagcagtctcctcaggaaccggactcctccgcgggtattggcaaatcgggtgcacagcccgctaaaaagagactcaattt |
| | cggtcagactggcgacacagagtcagtcccagaccctcaaccaatcggagaacctcccgcagccccctcaggtgtgggatctcttacaatggcttcag |
| | gtggtggcgcaccagtggcagacaataacgaaggtgccgatggagtgggtagttcctcgggaaattggcattgcgattcccaatggctgggggacag |
| | agtcatcaccaccagcacccgaacctgggccctgcccacctacaacaatcacctctacaagcaaatctccaacagcacatctggaggatcttcaaatga |
| | caacgcctacttcggctacagcaccccctgggggtattttgacttcaacagattccactgccacttctcaccacgtgactggcagcgactcatcaacaaca |
| | actggggattccggcctaagcgactcaacttcaagctcttcaacattcaggtcaaagaggttacggacaacaatggagtcaagaccatcgccaataacc |
| | ttaccagcacggtccaggtcttcacggactcagactatcagctcccgtacgtgctcgggtcggctcacgagggctgcctcccgccgttcccagcggac |
| | gttttcatgattcctcagtacgggtatctgacgcttaatgatggaagccaggccgtgggtcgttcgtccttttactgcctggaatatttcccgtcgcaaatgct |
| | aagaacgggtaacaacttccagttcagctacgagtttgagaacgtacctttccatagcagctacgctcacagccaaagcctggaccgactaatgaatcca |
| | ctcatcgaccaatacttgtactatctctcaaagactattaacggttctggacagaatcaacaaacgctaaaattcagtgtggccggacccagcaacatggc |
| | tgtccagggaagaaactacatacctggacccagctaccgacaacaacgtgtctcaaccactgtgactcaaaacaacaacagcgaatttgcttggcctgg |
| | agcttcttcttgggctctcaatggacgtaatagcttgatgaatcctggacctgctatggccagccacaaagaaggagaggaccgtttctttcctttgtctgg |
| | atctttaatttttggcaaacaaggaactggaagagacaacgtggatgcggacaaagtcatgataaccaacgaagaagaaattaaaactactaacccggt |
| | agcaacggagtcctatggacaagtggccacaaaccaccagagtgcccaagcacaggcgcagaccggctgggttcaaaaccaaggaatacttccgg |
| | gtatggtttggcaggacagagatgtgtacctgcaaggacccatttgggccaaaattcctcacacggacggcaactttcacccttctccgctgatgggagg |
| | gtttggaatgaagcacccgcctcctcagatcctcatcaaaaacacacctgtacctgcggatcctccaacggccttcaacaaggacaagctgaactctttc |
| | atcacccagtattctactggccaagtcagcgtggagatcgagtgggagctgcagaaggaaaacagcaagcgctggaacccggagatccagtacactt |
| | ccaactattacaagtctaataatgttgaatttgctgttaatactgaaggtgtatatagtgaaccccgccccattggcaccagatacctgactcgtaatctgtaa |
| | ttgcttgttaatcaataaaccgtttaattcgtttcagttgaactttggtctctgcgtatttctttcttatctagtttccatgctctaggcggccgcctcgagctgtgc |
| | cttctagttgccagccatctgttgtttgcccctcccccgtgccttccttgaccctggaaggtgccactcccactgtcctttcctaataaaatgaggaaattgca |
| | tcgcattgtctgagtaggtgtcattctattctggggggtggggggggcaggacagcaagggggaggattgggaagacaatagcaggcatgctgggg |
| | agtcgacgcgccggcgtctagaaggaacccctagtgatggagttggccactccctctctgcgcgctcgctcgctcactgaggccgggcgaccaaagg |
| | tcgcccgacgcccgggctttgcccgggcggcctcagtgagcgagcgagcgcgcagagagggagtggccaacagctgcattaatgaatcctgcagg |
| | cggccaacgcgcggggagaggcggtttgcgtattgggcgctcttccgcttcctcgctcactgactcgctgcgctcggtcgttcggctgcggcgagcgg |
| | tatcagctcactcaaaggcggtaatacggttatccacagaatcaggggataacgcaggaaagaacatgtgagcaaaaggccagcaaaaggccagga |
| | accgtaaaaaggccgcgttgctggcgtttttccataggctccgcccccctgacgagcatcacaaaaatcgacgctcaagtcagaggtggcgaaacccg |
| | acaggactataaagataccaggcgtttccccctggaagctccctcgtgcgctctcctgttccgaccctgccgcttaccggatacctgtccgcctttctccct |
| | tcgggaagcgtggcgctttctcatagctcacgctgtaggtatctcagttcggtgtaggtcgttcgctccaagctgggctgtgtgcacgaaccccccgttca |
| | gcccgaccgctgcgccttatccggtaactatcgtcttgagtccaacccggtaagacacgacttatcgccactggcagcagccactggtaacaggattag |
| | cagagcgaggtatgtaggcggtgctacagagttcttgaagtggtggcctaactacggctacactagaagaacagtatttggtatctgcgctctgctgaag |
| | ccagttaccttcggaaaaagagttggtagctcttgatccggcaaacaaaccaccgctggtagcggtggtttttttgtttgcaagcagcagattacgcgcag |
| | aaaaaaaggatctcaagaagatcctttgatcttttctacggggtctgacgctcagtggaacgaaaactcacgttaagggattttggtcatgacactacgtgt |
| | taccaatgcttaatcagtgaggcacctatctcagcgatctgtctatttcgttcatccatagttgcctgactccccgtcgtgtagataactacgatacgggagg |
| | gcttaccatctggccccagtgctgcaatgataccgcgagacccacgctcaccggctccagatttatcagcaataaaccagccagccggaagggccga |
| | gcgcagaagtggtcctgcaactttatccgcctccatccagtctattaattgttgccgggaagctagagtaagtagttcgccagttaatagtttgcgcaacgtt |
| | gttgccattgctacaggcatcgtggtgtcacgctcgtcgtttggtatggcttcattcagctccggttcccaacgatcaaggcgagttacatgatcccccatg |
| | ttgtgcaaaaaagcggttagctccttcggtcctccgatcgttgtcagaagtaagttggccgcagtgttatcactcatggttatggcagcactgcataattctc |
| | ttactgtcatgccatccgtaagatgcttttctgtgactggtgagtactcaaccaagtcattctgagaatagtgtatgcggcgaccgagttgctcttgcccggc |
| | gtcaatacgggataataccgcgccacatagcagaactttaaaagtgctcatcattggaaaacgttcttcggggcgaaaactctcaaggatcttaccgctgt |
| | tgagatccagttcgatgtaacccactcgtgcacccaactgatcttcagcatcttttactttcaccagcgtttctgggtgagcaaaaacaggaaggcaaaatg |
| | ccgcaaaaaagggaataagggcgacacggaaatgttgaatactcatactcttcctttttcaatattattgaagcatttatcagggttattgtctctatgagcgg |
| | atacatatttgaatgtatttagaaaaataaacaaataggggttccgcgcactacgtgggatcctcatgagattatcaaaaaggatcttcacctagatccttttc |
| | acgtagaaagccagtccgcagaaacggtgctgaccccctgaggcggaaagaaccagctgtggaatgtgtgtcagttagggtgtggaaagtccccag |
| | gctccccagcaggcagaagtatgcaaagcatgcatctcaattagtcagcaaccaggtgtggaaagtccccaggctccccagcaggcagaagtatgca |
| | aagcatgcatctcaattagtcagcaaccatagtcccgcccctaactccgcccatcccgcccctaactccgcccagttccgcccattctccgccccatggc |
| | tgactaattttttttatttatgcagaggccgaggccgcctcggcctctgagctattccagaagtagtgaggaggcttttttggaggcctaggcttttgcaaag |
| | gatgaatgtcagctactgggctatctggacaagggaaaacgcaagcgcaaagagaaagcaggtagcttgcagtgggcttacatggcgatagctagac |
| | tgggcggttttatggacagcaagcgaaccggaattgccagctggggcgccctctggtaaggttgggaagccctgcaaagtaaactggatggctttcttg |
| | ccgccaaggatctgatggcgcaggggatcaagctctgatcaagagacaggatgaggatcgtttcgcatgattgaacaagatggattgcacgcaggttc |
| | tccggccgcttgggtggagaggctattcggctatgactgggcacaacagacaatcggctgctctgatgccgccgtgttccggctgtcagcgcaggggc |
| | gcccggttctttttgtcaagaccgacctgtccggtgccctgaatgaactgcaagacgaggcagcgcggctatcgtggctggccacgacgggcgttcctt |
| | gcgcagctgtgctcgacgttgtcactgaagcgggaagggactggctgctattgggcgaagtgccggggcaggatctcctgtcatctcaccttgctcctg |
| | ccgagaaagtatccatcatggctgatgcaatgcggcggctgcatacgcttgatccggctacctgcccattcgaccaccaagcgaaacatcgcatcgag |
| | cgagcacgtactcggatggaagccggtcttgtcgatcaggatgatctggacgaagagcatcaggggctcgcgccagccgaactgttcgccaggctca |
| | aggcgagcatgcccgacggcgaggatctcgtcgtgacccatggcgatgcctgcttgccgaatatcatggtggaaaatggccgcttttctggattcatcg |
| | actgtggccggctgggtgtggcggaccgctatcaggacatagcgttggctacccgtgatattgctgaagagcttggcggcgaatgggctgaccgcttc |
| | ctcgtgctttacggtatcgccgctcccgattcgcagcgcatcgccttctatcgccttcttgacgagttcttctgaattttcacacaaaaaaccaacacacaga |
| | tgtaatgaaaataaagatattttattgttaaaatttttgttaaatcagctcattttttaaccaataggccgaaatcggcaaaatcccttataaatcaaaagaatag |
| | accgagatagggttgagtgttgttccagtttggaacaagagtccactattaaagaacgtggactccaacgtcaaagggcgaaaaaccgtctatcagggc |
| | gatggcccctcgaggaaccatcaccctaatcaagttttttggggtcgaggtgccgtaaagcactaaatcggaaccctaaagggagcccccgatttagag |
| | cttgacggggaaagccggcgaacgtggcgagaaaggaagggaagaaagcgaaaggagcgggcgctagggcgctggcaagtgtagcggtcacg |
| | ctgcgcgtaaccaccacacccgccgcgcttaatgcgccgctacagggcgcgtcgcgtccattcgccattcaggctgcgcaactgttgggaagggctg |
| | cag |
| 11 | pT7-CapX VP1 |
| | taatacgactcactataggatggctgccgatggttatcttccagattggctcgaggacaaccttagtgaaggaattcgcgagtggtgggctttgaaacctg |
| | gagcccctcaacccaaggcaaatcaacaacatcaagacaacgctcgaggtcttgtgcttccgggttacaaataccttggacccggcaacggactcgac |
| | aagggggagccggtcaacgcagcagacgcggcggccctcgagcacgacaaggcctacgaccagcagctcaaggccggagacaacccgtacctc |
| | aagtacaaccacgccgacgccgagttccaggagcggctcaaagaagatacgtcttttgggggcaacctcgggcgagcagtcttccaggccaaaaag |
| | aggcttcttgaacctcttggtctggttgaggaagcggctaagacggctcctggaaagaagaggcctgtagagcagtctcctcaggaaccggactcctcc |
| | gcgggtattggcaaatcgggtgcacagcccgctaaaaagagactcaatttcggtcagactggcgacacagagtcagtcccagaccctcaaccaatcg |
| | gagaacctcccgcagccccctcaggtgtgggatctcttacaatggcttcaggtggtggcgcaccagtggcagacaataacgaaggtgccgatggagt |
| | gggtagttcctcgggaaattggcattgcgattcccaatggctgggggacagagtcatcaccaccagcacccgaacctgggccctgcccacctacaaca |
| | atcacctctacaagcaaatctccaacagcacatctggaggatcttcaaatgacaacgcctacttcggctacagcaccccctgggggtattttgacttcaac |
| | agattccactgccacttctcaccacgtgactggcagcgactcatcaacaacaactggggattccggcctaagcgactcaacttcaagctcttcaacattca |
| | ggtcaaagaggttacggacaacaatggagtcaagaccatcgccaataaccttaccagcacggtccaggtcttcacggactcagactatcagctcccgta |
| | cgtgctcgggtcggctcacgagggctgcctcccgccgttcccagcggacgttttcatgattcctcagtacgggtatctgacgcttaatgatggaagccag |
| | gccgtgggtcgttcgtccttttactgcctggaatatttcccgtcgcaaatgctaagaacgggtaacaacttccagttcagctacgagtttgagaacgtacctt |
| | tccatagcagctacgctcacagccaaagcctggaccgactaatgaatccactcatcgaccaatacttgtactatctctcaaagactattaacggttctggac |
| | agaatcaacaaacgctaaaattcagtgtggccggacccagcaacatggctgtccagggaagaaactacatacctggacccagctaccgacaacaacg |
| | tgtctcaaccactgtgactcaaaacaacaacagcgaatttgcttggcctggagcttcttcttgggctctcaatggacgtaatagcttgatgaatcctggacct |
| | gctatggccagccacaaagaaggagaggaccgtttctttcctttgtctggatctttaatttttggcaaacaaggaactggaagagacaacgtggatgcgg |
| | acaaagtcatgataaccaacgaagaagaaattaaaactactaacccggtagcaacggagtcctatggacaagtggccacaaaccaccagagtgccca |
| | agcacaggcgcagaccggctgggttcaaaaccaaggaatacttccgggtatggtttggcaggacagagatgtgtacctgcaaggacccatttgggcc |
| | aaaattcctcacacggacggcaactttcacccttctccgctgatgggagggtttggaatgaagcacccgcctcctcagatcctcatcaaaaacacacctg |
| | tacctgcggatcctccaacggccttcaacaaggacaagctgaactctttcatcacccagtattctactggccaagtcagcgtggagatcgagtgggagct |
| | gcagaaggaaaacagcaagcgctggaacccggagatccagtacacttccaactattacaagtctaataatgttgaatttgctgttaatactgaaggtgtat |
| | atagtgaaccccgccccattggcaccagatacctgactcgtaatctgtaaaaaaaaaaaaaaaaaaaaaaaaaaaaaaaatgaagagccgtacgggc |
| | gcgcctaggcgcgattccgcttcctcgctcactgactcgctgcgctcggtcgttcggctgcggcgagcggtatcagctcactcaaaggcggtaatacg |
| | gttatccacagaatcaggggataacgcaggaaagaacatgtgagcaaaaggccagcaaaaggccaggaaccgtaaaaaggccgcgttgctggcgtt |
| | tttccataggctccgcccccctgacgagcatcacaaaaatcgacgctcaagtcagaggtggcgaaacccgacaggactataaagataccaggcgtttc |
| | cccctggaagctccctcgtgcgctctcctgttccgaccctgccgcttaccggatacctgtccgcctttctcccttcgggaagcgtggcgctttctcatagct |
| | cacgctgtaggtatctcagttcggtgtaggtcgttcgctccaagctgggctgtgtgcacgaaccccccgttcagcccgaccgctgcgccttatccggtaa |
| | ctatcgtcttgagtccaacccggtaagacacgacttatcgccactggcagcagccactggtaacaggattagcagagcgaggtatgtaggcggtgcta |
| | cagagttcttgaagtggtggcctaactacggctacactagaagaacagtatttggtatctgcgctctgctgaagccagttaccttcggaaaaagagttggt |
| | agctcttgatccggcaaacaaaccaccgctggtagcggtggtttttttgtttgcaagcagcagattacgcgcagaaaaaaaggatccaagaagatccttt |
| | gatcttttctacggggtctgacgctcagtggaacgaaaactcacgttaagggattttggtcatgagattatcaaaaaggatcttcacctagatccttttaaatt |
| | aaaaatgaagttttaaatcaatctaaagtatatatgagtaaacttggtctgacagttaccaatgcttaatcagtgaggcacctatctcagcgatctgtctatttc |
| | gttcatccatagttgcctgactccccgtcgtgtagataactacgatacgggagggcttaccatctggccccagtgctgcaatgataccgcgagatccacg |
| | ctcaccggctccagatttatcagcaataaaccagccagccggaagggccgagcgcagaagtggtcctgcaactttatccgcctccatccagtctattaat |
| | tgttgccgggaagctagagtaagtagttcgccagttaatagtttgcgcaacgttgttgccattgctacaggcatcgtggtgtcacgctcgtcgtttggtatg |
| | gcttcattcagctccggttcccaacgatcaaggcgagttacatgatcccccatgttgtgcaaaaaagcggttagctccttcggtcctccgatcgttgtcaga |
| | agtaagttggccgcagtgttatcactcatggttatggcagcactgcataattctcttactgtcatgccatccgtaagatgcttttctgtgactggtgagtactca |
| | accaagtcattctgagaatagtgtatgcggcgaccgagttgctcttgcccggcgtcaatacgggataataccgcgccacatagcagaactttaaaagtgc |
| | tcatcattggaaaacgttcttcggggcgaaaactctcaaggatcttaccgctgttgagatccagttcgatgtaacccactcgtgcacccaactgatcttcag |
| | catcttttactttcaccagcgtttctgggtgagcaaaaacaggaaggcaaaatgccgcaaaaaagggaataagggcgacacggaaatgttgaatactca |
| | tactcttcctttttcaatattattgaagcatttatcagggttattgtctcatgagcggatacatatttgaatgtatttagaaaaataaacaaataggggttccgcgc |
| | acatttccccgaaaagtgccacctgacgtctaagaaaccattattatcatgacattaacctataaaaataggcgtatcacgaggccctttcgtc |
| 12 | pLV-EF1A-VPX-WPRE-CMV-GFP |
| | aatgtagtcttatgcaatactcttgtagtcttgcaacatggtaacgatgagttagcaacatgccttacaaggagagaaaaagcaccgtgcatgccgattggt |
| | ggaagtaaggtggtacgatcgtgccttattaggaaggcaacagacgggtctgacatggattggacgaaccactgaattgccgcattgcagagatattgt |
| | atttaagtgcctagctcgatacataaacgggtctctctggttagaccagatctgagcctgggagctctctggctaactagggaacccactgcttaagcctc |
| | aataaagcttgccttgagtgcttcaagtagtgtgtgcccgtctgttgtgtgactctggtaactagagatccctcagacccttttagtcagtgtggaaaatctct |
| | agcagtggcgcccgaacagggacttgaaagcgaaagggaaaccagaggagctctctcgacgcaggactcggcttgctgaagcgcgcacggcaag |
| | aggcgaggggcggcgactggtgagtacgccaaaaattttgactagcggaggctagaaggagagagatgggtgcgagagcgtcagtattaagcggg |
| | ggagaattagatcgcgatgggaaaaaattcggttaaggccagggggaaagaaaaaatataaattaaaacatatagtatgggcaagcagggagctaga |
| | acgattcgcagttaatcctggcctgttagaaacatcagaaggctgtagacaaatactgggacagctacaaccatcccttcagacaggatcagaagaactt |
| | agatcattatataatacagtagcaaccctctattgtgtgcatcaaaggatagagataaaagacaccaaggaagctttagacaagatagaggaagagcaa |
| | aacaaaagtaagaccaccgcacagcaagcggccgctgatcttcagacctggaggaggagatatgagggacaattggagaagtgaattatataaatata |
| | aagtagtaaaaattgaaccattaggagtagcacccaccaaggcaaagagaagagtggtgcagagagaaaaaagagcagtgggaataggagctttgtt |
| | ccttgggttcttgggagcagcaggaagcactatgggcgcagcgtcaatgacgctgacggtacaggccagacaattattgtctggtatagtgcagcagc |
| | agaacaatttgctgagggctattgaggcgcaacagcatctgttgcaactcacagtctggggcatcaagcagctccaggcaagaatcctggctgtggaa |
| | agatacctaaaggatcaacagctcctggggatttggggttgctctggaaaactcatttgcaccactgctgtgccttggaatgctagttggagtaataaatct |
| | ctggaacagatttggaatcacacgacctggatggagtgggacagagaaattaacaattacacaagcttaatacactccttaattgaagaatcgcaaaacc |
| | agcaagaaaagaatgaacaagaattattggaattagataaatgggcaagtttgtggaattggtttaacataacaaattggctgtggtatataaaattattcat |
| | aatgatagtaggaggcttggtaggtttaagaatagtttttgctgtactttctatagtgaatagagttaggcagggatattcaccattatcgtttcagacccacct |
| | cccaaccccgaggggacccgacaggcccgaaggaatagaagaagaaggtggagagagagacagagacagatccattcgattagtgaacggatctc |
| | gacggtatcgctagcttttaaaagaaaaggggggattggggggtacagtgcaggggaaagaatagtagacataatagcaacagacatacaaactaaa |
| | gaattacaaaaacaaattacaaaaattcaaaattttactagtgattatcggatcaactttgtatagaaaagttgggctccggtgcccgtcagtgggcagagc |
| | gcacatcgcccacagtccccgagaagttggggggaggggtcggcaattgaaccggtgcctagagaaggtggcgcggggtaaactgggaaagtgat |
| | gtcgtgtactggctccgcctttttcccgaggggggggagaaccgtatataagtgcagtagtcgccgtgaacgttctttttcgcaacgggtttgccgccag |
| | aacacaggtaagtgccgtgtgtggttcccgcgggcctggcctctttacgggttatggcccttgcgtgccttgaattacttccacctggctgcagtacgtgat |
| | tcttgatcccgagcttcgggttggaagtgggtgggagagttcgaggccttgcgcttaaggagccccttcgcctcgtgcttgagttgaggcctggcctgg |
| | gcgctggggccgccgcgtgcgaatctggtggcaccttcgcgcctgtctcgctgctttcgataagtctctagccatttaaaatttttgatgacctgctgcgac |
| | gctttttttctggcaagatagtcttgtaaatgcgggccaagatctgcacactggtatttcggtttttggggccgcgggcggcgacggggcccgtgcgtccc |
| | agcgcacatgttcggcgaggcggggcctgcgagcgcggccaccgagaatcggacgggggtagtctcaagctggccggcctgctctggtgcctggt |
| | ctcgcgccgccgtgtatcgccccgccctgggcggcaaggctggcccggtcggcaccagttgcgtgagcggaaagatggccgcttcccggccctgct |
| | gcagggagctcaaaalggaggacgcggcgctcgggagagcggggggtgagtcacccacacaaaggaaaagggcctttccgtcctcagccgtcg |
| | cttcatgtgactccacggagtaccgggcgccgtccaggcacctcgattagttctcgagcttttggagtacgtcgtctttaggttggggggaggggttttat |
| | gcgatggagtttccccacactgagtgggtggagactgaagttaggccagcttggcacttgatgtaattctccttggaatttgccctttttgagtttggatcttg |
| | gttcattctcaagcctcagacagtggttcaaagtttttttcttccatttcaggtgtcgtgacaagtttgtacaaaaaagcaggctgccaccatggctgccgatg |
| | gttatcttccagattggctcgaggacaaccttagtgaaggaattcgcgagtggtgggctttgaaacctggagcccctcaacccaaggcaaatcaacaac |
| | atcaagacaacgctcgaggtcttgtgcttccgggttacaaataccttggacccggcaacggactcgacaagggggagccggtcaacgcagcagacg |
| | cggcggccctcgagcacgacaaggcctacgaccagcagctcaaggccggagacaacccgtacctcaagtacaaccacgccgacgccgagttcca |
| | ggagcggctcaaagaagatacgtcttttgggggcaacctcgggcgagcagtcttccaggccaaaaagaggcttcttgaacctcttggtctggttgagga |
| | agcggctaagacggctcctggaaagaagaggcctgtagagcagtctcctcaggaaccggactcctccgcgggtattggcaaatcgggtgcacagcc |
| | cgctaaaaagagactcaatttcggtcagactggcgacacagagtcagtcccagaccctcaaccaatcggagaacctcccgcagccccctcaggtgtg |
| | ggatctcttacaatggcttcaggtggtggcgcaccagtggcagacaataacgaaggtgccgatggagtgggtagttcctcgggaaattggcattgcgat |
| | tcccaatggctgggggacagagtcatcaccaccagcacccgaacctgggccctgcccacctacaacaatcacctctacaagcaaatctccaacagca |
| | catctggaggatcttcaaatgacaacgcctacttcggctacagcaccccctgggggtattttgacttcaacagattccactgccacttctcaccacgtgact |
| | ggcagcgactcatcaacaacaactggggattccggcctaagcgactcaacttcaagctcttcaacattcaggtcaaagaggttacggacaacaatgga |
| | gtcaagaccatcgccaataaccttaccagcacggtccaggtcttcacggactcagactatcagctcccgtacgtgctcgggtcggctcacgagggctg |
| | cctcccgccgttcccagcggacgttttcatgattcctcagtacgggtatctgacgcttaatgatggaagccaggccgtgggtcgttcgtccttttactgcct |
| | ggaatatttcccgtcgcaaatgctaagaacgggtaacaacttccagttcagctacgagtttgagaacgtacctttccatagcagctacgctcacagccaaa |
| | gcctggaccgactaatgaatccactcatcgaccaatacttgtactatctctcaaagactattaacggttctggacagaatcaacaaacgctaaaattcagtg |
| | lggccggacccagcaacatggctgtccagggaagaaactacatacctggacccagctaccgacaacaacgtgtctcaaccactgtgactcaaaacaa |
| | caacagcgaatttgcttggcctggagcttcttcttgggctctcaatggacgtaatagcttgatgaatcctggacctgctatggccagccacaaagaagga |
| | gaggaccgtttctttcctttgtctggatctttaatttttggcaaacaaggaactggaagagacaacgtggatgcggacaaagtcatgataaccaacgaaga |
| | agaaattaaaactactaacccggtagcaacggagtcctatggacaagtggccacaaaccaccagagtgcccaagcacaggcgcagaccggctgggt |
| | tcaaaaccaaggaatacttccgggtatggtttggcaggacagagatgtgtacctgcaaggacccatttgggccaaaattcctcacacggacggcaacttt |
| | cacccttctccgctgatgggagggtttggaatgaagcacccgcctcctcagatcctcatcaaaaacacacctgtacctgcggatcctccaacggccttca |
| | acaaggacaagctgaactctttcatcacccagtattctactggccaagtcagcgtggagatcgagtgggagctgcagaaggaaaacagcaagcgctg |
| | gaacccggagatccagtacacttccaactattacaagtctaataatgttgaatttgctgttaatactgaaggtgtatatagtgaaccccgccccattggcacc |
| | agatacctgactcgtaatctgtaaacccagctttcttgtacaaagtggtgataatcgaattccgataatcaacctctggattacaaaatttgtgaaagattgac |
| | tggtattcttaactatgttgctccttttacgctatgtggatacgctgctttaatgcctttgtatcatgctattgcttcccgtatggctttcattttctcctccttgtataa |
| | atcctggttgctgtctctttatgaggagttgtggcccgttgtcaggcaacgtggcgtggtgtgcactgtgtttgctgacgcaacccccactggttggggcat |
| | tgccaccacctgtcagctcctttccgggactttcgctttccccctccctattgccacggcggaactcatcgccgcctgccttgcccgctgctggacaggg |
| | gctcggctgttgggcactgacaattccgtggtgttgtcggggaagctgacgtcctttccatggctgctcgcctgtgttgccacctggattctgcgcgggac |
| | gtccttctgctacgtcccttcggccctcaatccagcggaccttccttcccgcggcctgctgccggctctgcggcctcttccgcgtcttcgccttcgccctca |
| | gacgagtcggatctccctttgggccgcctccccgcatcgggaattcccgcggttcgaacgcgttgacattgattattgactagttattaatagtaatcaatta |
| | cggggtcattagttcatagcccatatatggagttccgcgttacataacttacggtaaatggcccgcctggctgaccgcccaacgacccccgcccattgac |
| | gtcaataatgacgtatgttcccatagtaacgccaatagggactttccattgacgtcaatgggtggagtatttacggtaaactgcccacttggcagtacatca |
| | agtgtatcatatgccaagtacgccccctattgacgtcaatgacggtaaatggcccgcctggcattatgcccagtacatgaccttatgggactttcctacttg |
| | gcagtacatctacgtattagtcatcgctattaccatggtgatgcggttttggcagtacatcaatgggcgtggatagcggtttgactcacggggatttccaag |
| | tctccaccccattgacgtcaatgggagtttgttttggcaccaaaatcaacgggactttccaaaatgtcgtaacaactccgccccattgacgcaaatgggcg |
| | gtaggcgtgtacggtgggaggtctatataagcagagctctctggctaactagagaacccactgcgccaccatggtgagcaagggcgaggagctgttc |
| | accggggtggtgcccatcctggtcgagctggacggcgacgtaaacggccacaagttcagcgtgtccggcgagggcgagggcgatgccacctacgg |
| | caagctgaccctgaagttcatctgcaccaccggcaagctgcccgtgccctggcccaccctcgtgaccaccctgacctacggcgtgcagtgcttcagcc |
| | gctaccccgaccacatgaagcagcacgacttcttcaagtccgccatgcccgaaggctacgtccaggagcgcaccatcttcttcaaggacgacggcaa |
| | ctacaagacccgcgccgaggtgaagttcgagggcgacaccctggtgaaccgcatcgagctgaagggcatcgacttcaaggaggacggcaacatcct |
| | ggggcacaagctggagtacaactacaacagccacaacgtctatatcatggccgacaagcagaagaacggcatcaaggtgaacttcaagatccgccac |
| | aacatcgaggacggcagcgtgcagctcgccgaccactaccagcagaacacccccatcggcgacggccccgtgctgctgcccgacaaccactacct |
| | gagcacccagtccgccctgagcaaagaccccaacgagaagcgcgatcacatggtcctgctggagttcgtgaccgccgccgggatcactctcggcat |
| | ggacgagctgtacaagtaaggtacctttaagaccaatgacttacaaggcagctgtagatcttagccactttttaaaagaaaaggggggactggaagggc |
| | taattcactcccaacgaagacaagatctgctttttgcttgtactgggtctctctggttagaccagatctgagcctgggagctctctggctaactagggaacc |
| | cactgcttaagcctcaataaagcttgccttgagtgcttcaagtagtgtgtgcccgtctgttgtgtgactctggtaactagagatccctcagacccttttagtca |
| | gtgtggaaaatctctagcagtagtagttcatgtcatcttattattcagtatttataacttgcaaagaaatgaatatcagagagtgagaggaacttgtttattgca |
| | gcttataatggttacaaataaagcaatagcatcacaaatttcacaaataaagcatttttttcactgcattctagttgtggtttgtccaaactcatcaatgtatctta |
| | tcatgtctggctctagctatcccgcccctaactccgcccatcccgcccctaactccgcccagttccgcccattctccgccccatggctgactaattttttttat |
| | ttatgcagaggccgaggccgcctcggcctctgagctattccagaagtagtgaggaggcttttttggaggcctagggacgtacccaattcgccctatagtg |
| | agtcgtattacgcgcgctcactggccgtcgttttacaacgtcgtgactgggaaaaccctggcgttacccaacttaatcgccttgcagcacatccccctttc |
| | gccagctggcgtaatagcgaagaggcccgcaccgatcgcccttcccaacagttgcgcagcctgaatggcgaatgggacgcgccctgtagcggcgc |
| | attaagcgcggcgggtgtggtggttacgcgcagcgtgaccgctacacttgccagcgccctagcgcccgctcctttcgctttcttcccttcctttctcgcca |
| | cgttcgccggctttccccgtcaagctctaaatcgggggctccctttagggttccgatttagtgctttacggcacctcgaccccaaaaaacttgattagggtg |
| | atggttcacgtagtgggccatcgccctgatagacggtttttcgccctttgacgttggagtccacgttctttaatagtggactcttgttccaaactggaacaac |
| | actcaaccctatctcggtctattcttttgatttataagggattttgccgatttcggcctattggttaaaaaatgagctgatttaacaaaaatttaacgcgaatttta |
| | acaaaatattaacgcttacaatttaggtggcacttttcggggaaatgtgcgcggaacccctatttgtttatttttctaaatacattcaaatatgtatccgctcatg |
| | agacaataaccctgataaatgcttcaataatattgaaaaaggaagagtatgagtattcaacatttccgtgtcgcccttattcccttttttgcggcattttgccttc |
| | ctgtttttgctcacccagaaacgctggtgaaagtaaaagatgctgaagatcagttgggtgcacgagtgggttacatcgaactggatctcaacagcggtaa |
| | gatccttgagagttttcgccccgaagaacgttttccaatgatgagcacttttaaagttctgctatgtggcgcggtattatcccgtattgacgccgggcaaga |
| | gcaactcggtcgccgcatacactattctcagaatgacttggttgagtactcaccagtcacagaaaagcatcttacggatggcatgacagtaagagaattat |
| | gcagtgctgccataaccatgagtgataacactgcggccaacttacttctgacaacgatcggaggaccgaaggagctaaccgcttttttgcacaacatgg |
| | gggatcatgtaactcgccttgatcgttgggaaccggagctgaatgaagccataccaaacgacgagcgtgacaccacgatgcctgtagcaatggcaac |
| | aacgttgcgcaaactattaactggcgaactacttactctagcttcccggcaacaattaatagactggatggaggcggataaagttgcaggaccacttctgc |
| | gctcggcccttccggctggctggtttattgctgataaatctggagccggtgagcgtgggtctcgcggtatcattgcagcactggggccagatggtaagc |
| | cctcccgtatcgtagttatctacacgacggggagtcaggcaactatggatgaacgaaatagacagatcgctgagataggtgcctcactgattaagcattg |
| | gtaactgtcagaccaagtttactcatatatactttagattgatttaaaacttcatttttaatttaaaaggatctaggtgaagatcctttttgataatctcatgaccaa |
| | aatcccttaacgtgagttttcgttccactgagcgtcagaccccgtagaaaagatcaaaggatcttcttgagatcctttttttctgcgcgtaatctgctgcttgca |
| | aacaaaaaaaccaccgctaccagcggtggtttgtttgccggatcaagagctaccaactctttttccgaaggtaactggcttcagcagagcgcagatacca |
| | aatactgttcttctagtgtagccgtagttaggccaccacttcaagaactctgtagcaccgcctacatacctcgctctgctaatcctgttaccagtggctgctg |
| | ccagtggcgataagtcgtgtcttaccgggttggactcaagacgatagttaccggataaggcgcagcggtcgggctgaacggggggttcgtgcacaca |
| | gcccagcttggagcgaacgacctacaccgaactgagatacctacagcgtgagctatgagaaagcgccacgcttcccgaagagagaaaggcggaca |
| | ggtatccggtaagcggcagggtcggaacaggagagcgcacgagggagcttccagggggaaacgcctggtatctttatagtcctgtcgggtttcgcca |
| | cctctgacttgagcgtcgatttttgtgatgctcgtcaggggggcggagcctatggaaaaacgccagcaacgcggcctttttacggttcctggccttttgct |
| | ggccttttgctcacatgttctttcctgcgttatcccctgattctgtggataaccgtattaccgcctttgagtgagctgataccgctcgccgcagccgaacgac |
| | cgagcgcagcgagtcagtgagcgaggaagcggaagagcgcccaatacgcaaaccgcctctccccgcgcgttggccgattcattaatgcagctggca |
| | cgacaggtttcccgactggaaagcgggcagtgagcgcaacgcaattaatgtgagttagctcactcattaggcaccccaggctttacactttatgcttccg |
| | gctcgtatgttgtgtggaattgtgagcggataacaatttcacacaggaaacagctatgaccatgattacgccaagcgcgcaattaaccctcactaaaggg |
| | aacaaaagctggagctgcaagctt |

What is claimed is:

1. A method of inducing immune tolerance to a therapeutic recombinant adeno-associated virus (rAAV) in a subject comprising: administering to the subject an effective amount of a tolerance-inducing gene therapy vector,
wherein the tolerance-inducing gene therapy vector comprises a nucleic acid sequence encoding at least a portion of a capsid protein of the therapeutic rAAV, wherein the nucleic acid sequence is operably linked to a promoter; and
wherein the tolerance-inducing gene therapy vector is capable delivering the nucleic acid sequence to a liver cell or hematopoietic stem cell (HSC) and expressing the at least a portion of the capsid protein of the therapeutic rAAV in the liver or HSC, thereby inducing immune tolerance to the therapeutic rAAV.

2. The method of claim 1, wherein the promoter is a constitutive promoter.

3. The method of claim 2, wherein the promoter comprises a Herpes Simplex virus (HSV) promoter, a thymidine kinase (TK) promoter, a Rous Sarcoma Virus (RSV) promoter, a Simian Virus 40 (SV40) promoter, a Mouse Mammary Tumor Virus (MMTV) promoter, an Adenovirus E1A promoter, a cytomegalovirus (CMV) promoter, a mammalian housekeeping gene promoter, or a β-actin promoter.

4. The method of claim 1, wherein the promoter comprises an inducible promoter.

5. The method of claim 4, wherein the inducible promoter comprises a cytochrome P450 gene promoter, a heat shock protein gene promoter, a metallothionein gene promoter, a hormone-inducible gene promoter, an estrogen gene promoter, or a tetVP16 promoter that is responsive to tetracycline.

6. The method of claim 1 or 2, wherein the promoter comprises a liver-specific promoter, a hepatocyte-specific promoter, or a HSC-specific promoter.

7. The method of claim 6, wherein the liver-specific promoter comprises an albumin promoter, an alpha-1-antitrypsin promoter, or a hepatitis B virus core protein promoter.

8. The method of any one of claims 1-7, wherein the promoter comprises a synthetic promoter.

9. The method of claim 1, wherein the promoter comprises the nucleic acid sequence of SEQ ID NO: 3, or a nucleic acid sequence having at least 90% sequence identity to SEQ ID NO: 3.

10. The method of any one of claims 1-9, wherein the at least a portion of the capsid protein of the therapeutic rAAV comprises an immunogenic fragment of the capsid protein.

11. The method of claim 10, wherein the at least a portion of the capsid protein of the therapeutic rAAV comprises at least 10% at least 20%, at least 30%, at least 40%, at least 50%, at least 60%, at least 70%, at least 80%, at least 90%, or 100% of the capsid protein.

12. The method of claim 10, wherein the immunogenic fragment of the capsid protein comprises an immunogenic portion of one or more of: a VP1 capsid protein, a VP2 capsid protein, and a VP3 capsid protein.

13. The method of claim 12, wherein the immunogenic fragment of the capsid protein comprises an immunogenic portion of the VP3 capsid protein.

14. The method of claim 13, wherein the immunogenic fragment of the capsid protein has at least 90% identity to the amino acid sequence of SEQ ID NO: 1.

15. The method of claim 14, wherein the immunogenic fragment of the capsid protein is encoded by a nucleic acid sequence having at least 90% identify to SEQ ID NO: 2.

16. The method of any one of claims 1-15, wherein the tolerance-inducing gene therapy vector is a non-viral vector.

17. The method of claim 16, wherein the non-viral vector comprises an lipid nanoparticle.

18. The method of any one of claims 1-15, wherein the tolerance-inducing gene therapy vector is a lentiviral vector.

20. The method of any one of claims 1-19, wherein the therapeutic rAAV is an AAV of serotype 1, serotype 2, serotype 3, serotype 4, serotype 5, serotype 6, serotype 7, serotype 8, serotype 9, serotype 10, serotype 11, rh10, or rh74.

21. The method of any one of claims 1-20, wherein the tolerance-inducing gene therapy vector is administered systemically.

22. The method of any one of claims 1-21, wherein the subject has previously been administered the therapeutic rAAV or the subject has not been previously administered the therapeutic rAAV.

23. The method of any one of claims 1-22, wherein inducing immune tolerance comprises one or more of:
(a) inducing therapeutic rAAV-specific regulatory T cells (Tregs),
(b) reducing cytotoxic CD8+ T cell response to the therapeutic rAAV,
(c) reducing the level of pre-existing antibodies to the therapeutic rAAV, and
(d) reduce production of antibodies against the therapeutic rAAV.

24. A method for delivering a therapeutic nucleic acid to in a subject comprising
(a) administering to the subject an effective amount of a therapeutic recombinant adeno-associated virus (rAAV) comprising a first nucleic acid comprising the therapeutic nucleic acid operably linked to a first promoter; and
(b) administering to the subject an effective amount of a tolerance-inducing gene therapy vector comprising a second nucleic acid sequence encoding at least a portion of a capsid protein of the therapeutic rAAV, wherein the second nucleic acid sequence is operably linked to a second promoter; and wherein the tolerance-inducing gene therapy vector is capable delivering the nucleic acid sequence to a liver cell or HSC and expressing the at least a portion of the capsid protein of the therapeutic rAAV in the liver or HSC, thereby inducing immune tolerance to the therapeutic rAAV.

25. The method of claim 25, wherein therapeutic nucleic acid encodes an antigen, a therapeutic protein, or a therapeutic RNA.

26. The method of claim 24 or 25, wherein the first promoter and/or the second promoter are constitutive promoters.

27. The method of claim 26, wherein the first promoter and/or the second promoter independently comprise a Herpes Simplex virus (HSV) promoter, a thymidine kinase (TK) promoter, a Rous Sarcoma Virus (RSV) promoter, a Simian Virus 40 (SV40) promoter, a Mouse Mammary Tumor Virus (MMTV) promoter, an Adenovirus E1A promoter, a cytomegalovirus (CMV) promoter, a mammalian housekeeping gene promoter, or a β-actin promoter.

28. The method of claim 24, wherein the first promoter and/or the second promoter comprise an inducible promoter.

29. The method of claim 28, wherein the inducible promoter comprises a cytochrome P450 gene promoter, a heat shock protein gene promoter, a metallothionein gene promoter, a hormone-inducible gene promoter, an estrogen gene promoter, or a tetVP16 promoter that is responsive to tetracycline.

30. The method of claim 24, wherein the first promoter and/or the second promoter comprise tissue-specific promoters.

31. The method of claim 30, wherein the tissue-specific promoter comprises a neuronal-specific promoter, a muscle-specific promoter, or a liver-specific promoter.

32. The method of claim 31, wherein the first promoter comprises a muscle-specific promoter selected from the group consisting of: a desmin promoter, a creatine kinase promoter, a myogenin promoter, an alpha myosin heavy chain promoter and a natriuretic peptide promoter.

33. The method of claim 31, wherein the second promoter comprises a liver-specific promoter, a hepatocyte-specific promoter, or a HSC-specific promoter.

34. The method of claim 32, wherein the liver-specific promoter comprises an albumin promoter, an alpha-1-antitrypsin promoter, or a hepatitis B virus core protein promoter.

35. The method of any one of claims 24-34, wherein the first promoter and/or the second promoter comprise synthetic promoters.

36. The method of claim 36, wherein the second promoter comprises the nucleic acid sequence pf SEQ ID NO: 3, or a nucleic acid sequence having at least 90% sequence identity to SEQ ID NO: 3.

37. The method of any one of claims 24-36, wherein the at least a portion of the capsid protein of the therapeutic rAAV comprises an immunogenic fragment of the capsid protein.

38. The method of claim 37, wherein the at least a portion of the capsid protein of the therapeutic rAAV comprises at least 10% at least 20%, at least 30%, at least 40%, at least 50%, at least 60%, at least 70%, at least 80%, at least 90%, or 100% of the capsid protein.

39. The method of claim 38, wherein the immunogenic fragment of the capsid protein comprises an immunogenic portion of one or more of: a VP1 capsid protein, a VP2 capsid protein, and a VP3 capsid protein.

40. The method of claim 39, wherein the immunogenic fragment of the capsid protein (a) has at least 90% identity to the amino acid sequence of SEQ ID NO: 1; or (b) is encoded by a nucleic acid sequence having at least 90% identify to SEQ ID NO: 2.

41. The method of any one of claims 3-33, wherein the therapeutic rAAV is an AAV serotype selected from the group consisting of: serotype 1, serotype 2, serotype 3, serotype 4, serotype 5, serotype 6, serotype 7, serotype 8, serotype 9, serotype 10, serotype 11, rh 10 and rh74.

42. The method of any one of claims 22-42, wherein
(a) the therapeutic rAAV and the tolerance-inducing gene therapy vector are co-administered;
(b) the therapeutic rAAV is administered before the tolerance-inducing gene therapy vector; or
(c) the therapeutic rAAV is administered after the tolerance-inducing gene therapy vector.

43. The method of any one of claims 25-42, wherein the antigen comprises a viral antigen, a bacterial antigen, a parasite antigen, a fungal antigen, or a tumor antigen.

44. The method of any one of claims 24-43, further comprising administering to the subject a second effective amount of the therapeutic rAAV, wherein the second effective dose is administered after the first effective dose.

45. The method of any one of claims 25-44, wherein the therapeutic nucleic acid encodes an antigen.

46. The method of claim 45, wherein the method induces an immune response against the antigen.

47. The method of claim 46, wherein inducing an immune response comprises one or more of:
(a) eliciting a cellular immune response to the antigen;
(b) elicit a humoral immune response to the antigen;
(c) enhancing proliferation of antigen-specific cytotoxic T lymphocytes;
(d) eliciting generation of anti-antigen antibodies;
(e) reduce the likelihood of infection by pathogen containing the antigen;
(f) vaccinating the subject against the pathogen containing the antigen; and
(g) treating cancer.

48. A gene therapy vector comprising a nucleic acid sequence encoding an immunogenic portion of a capsid protein of an rAAV, wherein the nucleic acid sequence is operably linked to a promoter, and wherein the gene therapy vector is capable of delivering the nucleic acid sequence to liver cell or HSC and expressing the at least a portion of the capsid protein of the therapeutic rAAV in the liver or HSC, thereby inducing immune tolerance to the therapeutic rAAV.

49. The gene therapy vector of claim 48, wherein the gene therapy vector is a non-viral vector or a lentiviral vector.

50. A method for delivering a therapeutic nucleic acid to in a subject comprising
(a) administering to the subject an effective amount of a therapeutic recombinant adeno-associated virus (rAAV) comprising a first nucleic acid comprising the therapeutic nucleic acid operably linked to a first promoter; and
(b) administering to the subject an effective amount of a HSC expressing at least a portion of a capsid protein of the therapeutic rAAV, thereby inducing immune tolerance to the therapeutic rAAV.

51. A method of inducing immune tolerance to a therapeutic recombinant adeno-associated virus (rAAV) in a subject comprising: administering to the subject an effective amount of a HSC expressing at least a portion of a capsid protein of the therapeutic rAAV, thereby inducing immune tolerance to the therapeutic rAAV.


SEQUENCE LISTING (33 sequences)

SEQ ID NO: 1 - protein, 534 aa, Adeno associated virus
  REGION 1..534: note = MISC_FEATURE - VP3 Capsid Protein of AAV1
MASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 2 - DNA, 1605 bp, Adeno associated virus
  misc_feature 1..1605: note = AAV VP3 Capsid DNA
  source 1..1605: mol_type = other DNA
atggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 3 - DNA, 410 bp, synthetic construct
  misc_feature 1..410: note = Liver Specific Promoter
  misc_feature 1..410: note = Liver specific GAA Promoter
  source 1..410: mol_type = other DNA
ccctaaaatgggcaaacattgcaagcagcaaacagcaaacacacagccctccctgcctgctgaccttggagctggggcagaggtcagagacctctctggggactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgtagggccctgtctcctcagcttcaggcaccaccactgacctgggacagtgaatccgga

SEQ ID NO: 4 - DNA, 5603 bp, synthetic construct
  misc_feature 1..5603: note = pTR-LSP-AAV1-VP3
  source 1..5603: mol_type = other DNA
ctgcaggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctccctaaaatgggcaaacattgcaagcagcaaacagcaaacacacagccctccctgcctgctgaccttggagctggggcagaggtcagagacctctctggggactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgtagggccctgtctcctcagcttcaggcaccaccactgacctgggacagtgaatccggaaagtatcaaggttacaagacaggtttaaggagaccaatagaaactgggcttgtcgagacagagaagactcttgcgtttctgataggcacctattggtcttactgacatccactttgcctttctctccacagacgcgtggtaccgtcgacccgccaccatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaatctagagatatcgcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggg

SEQ ID NO: 5 - DNA, 3841 bp, synthetic construct
  misc_feature 1..3841: note = SARS-CoV-2 Spike Protein
  source 1..3841: mol_type = other DNA
agcttgaattcgccaccatgttcgtgtttctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacctgagc

SEQ ID NO: 6 - DNA, 8006 bp, synthetic construct
  misc_feature 1..8006: note = pTR-SARS2S-2P
  source 1..8006: mol_type = other DNA
gggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagcttgaattcgccaccatgttcgtgtttctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacctgagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagggctgcag

SEQ ID NO: 7 - DNA, 3730 bp, synthetic construct
  misc_feature 1..3730: note = SARS-CoV-2 Spike Protein deleted TM domain
  source 1..3730: mol_type = other DNA
agcttgaattcgccaccatgttcgtgtttctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagggctacatccctgaggctcctagagatggccaggcctacgtcagaaaggatggcgagtgggtcctgctgagcaccttcctgtaatctagagc

SEQ ID NO: 8 - DNA, 7895 bp, synthetic construct
  misc_feature 1..7895: note = p-TR-SARS2-2P-dTM
  source 1..7895: mol_type = other DNA
gggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagcttgaattcgccaccatgttcgtgtttctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagggctacatccctgaggctcctagagatggccaggcctacgtcagaaaggatggcgagtgggtcctgctgagcaccttcctgtaatctagagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagggctgcag

SEQ ID NO: 9 - DNA, 748 bp, synthetic construct
  misc_feature 1..748: note = CMV Promoter
  source 1..748: mol_type = other DNA
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactaga

SEQ ID NO: 10 - DNA, 7363 bp, synthetic construct
  misc_feature 1..7363: note = AAV plasmid sequence
  source 1..7363: mol_type = other DNA
ttaattaactgcagacgccagctgttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttctagaggcgcgccaagcttccctaaaatgggcaaacattgcaagcagcaaacagcaaacacacagccctccctgcctgctgaccttggagctggggcagaggtcagagacctctctggggactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgtagggccctgtctcctcagcttcaggcaccaccactgacctgggacagtgaatccggaactagtaagtatcaaggttacaagacaggtttaaggagaccaatagaaactgggcttgtcgagacagagaagactcttgcgtttctgataggcacctattggtcttactgacatccactttgcctttctctccacaggctagcatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgtatttctttcttatctagtttccatgctctaggcggccgcctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagtcgacgcgccggcgtctagaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaacagctgcattaatgaatcctgcaggcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacactacgtgttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctctatgagcggatacatatttgaatgtatttagaaaaataaacaaataggggttccgcgcactacgtgggatcctcatgagattatcaaaaaggatcttcacctagatccttttcacgtagaaagccagtccgcagaaacggtgctgaccccctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattttcacacaaaaaaccaacacacagatgtaatgaaaataaagatattttattgttaaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccctcgaggaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgtccattcgccattcaggctgcgcaactgttgggaagggctgcag

SEQ ID NO: 11 - DNA, 4291 bp, synthetic construct
  misc_feature 1..4291: note = pT7-CapX VP1
  source 1..4291: mol_type = other DNA
taatacgactcactataggatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaaaaaaaaaaaaaaaaaaaaaaaaaaaaaaaatgaagagccgtacgggcgcgcctaggcgcgattccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 12 - DNA, 10925 bp, synthetic construct
  misc_feature 1..10925: note = pLV-EF1A-VPX-WPRE-CMV-GFP
  source 1..10925: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgaacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 13 - DNA, 145 bp, Adeno associated virus
  source 1..145: mol_type = genomic DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 14 - DNA, 133 bp, synthetic construct
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 15 - DNA, 2211 bp, synthetic construct
  source 1..2211: mol_type = other DNA
atggccgccgacggctacctgcctgactggctggaagataatctgagcgagggcatccgggaatggtgggccctgaagcccggcgctcctcaacctaaggcaaaccagcagcaccaggacaacgccagaggcctcgtgctgcctggatataagtacctgggacctggcaacggcctggacaagggcgagcccgtgaacgccgctgatgccgctgctctggagcacgacaaggcctacgaccagcagctgaaagccggcgataacccctacctgaagtacaaccacgccgacgccgagttccaagagagactgaaggaagataccagctttggcggaaatctgggcagagccgtgttccaggccaagaagcgcctgctggaacctctgggactggtcgaggaagccgccaagacagcccctggcaagaaaagacctgtggaacagagccctcaggagcccgacagctccgccggaatcggcaaaagcggcgcccagcccgccaaaaagcggctgaacttcggccagaccggcgacacagagtctgtgccagatcctcagcctatcggcgagccacctgctgcccccagcggtgttggcagcctgaccatggcctctggcggcggcgccccagtggccgacaacaacgagggagctgacggcgtgggctcttccagcggcaattggcactgcgacagccaatggctgggagatagagtgatcaccaccagcacaagaacctgggctctgcctacatacaacaaccacctgtacaaacagatcagcaactccactagcggtggcagcagcaacgacaatgcctacttcggctacagcaccccttggggatacttcgactttaacagattccactgtcacttctctcctagagattggcagagactgatcaacaacaactggggcttccggcctaagcggcttaacttcaagctgttcaacatccaggtcaaagaggtgacagataacaatggagtgaagaccatcgccaacaacctgacaagcactgtgcaggtgttcaccgacagtgattaccagctgccatacgtgctgggctctgcccacgagggctgcctgcctcctttccctgctgacgtgttcatgatccctcagtatggctacctgaccctgaatgatggctcccaggccgtgggcagaagctccttctactgcctggaatactttcctagccaaatgctgagaacagggaacaacttccagtttagctacgagttcgagaacgtgcccttccacagcagctacgcccattctcaaagcctggacagactgatgaatcccctgatcgaccagtacctgtactacctcagcaagaccatcaacggcagtggccagaaccagcagaccctgaagttcagcgtggcgggcccttctaatatggcagtgcagggccgcaactacatcccaggcccctcctatagacagcagcgggtgtctacaaccgtgacccagaacaacaactcagagtttgcctggcctggcgcctctagctgggccctgaacggcagaaatagcctgatgaaccctggccccgctatggccagccacaaggaaggcgaggaccggttcttccccctgagcggctctctgattttcggcaagcagggcaccggcagggacaacgtggacgccgataaggtgatgatcaccaacgaggaagaaatcaagacaaccaatcctgtggccaccgagagctacggccaggtcgccacaaatcaccagagcgcccaggctcaggcccaaacaggctgggttcagaatcagggcatcctgcccggcatggtgtggcaggacagagatgtgtacctgcaaggccctatctgggccaagatcccccacaccgacggaaacttccaccccagccctctcatgggcggctttggcatgaagcaccctccaccacagatcctgatcaagaacacacctgtgccagccgatcctcctaccgcctttaacaaggacaagctgaacagcttcatcacacagtacagcaccggacaagtgtccgtggaaatcgagtgggagctgcagaaggaaaacagcaaaagatggaacccggaaattcagtacaccagcaactactacaaaagcaacaacgtggaattcgccgtgaacaccgagggagtgtattctgagcctcggcccatcggaaccagatacctgacccggaacctgtga

SEQ ID NO: 16 - protein, 736 aa, Adeno associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 17 - DNA, 88 bp, Adeno associated virus
  source 1..88: mol_type = genomic DNA
ttgcttgttaatcaataaaccgtttaattcgtttcagttgaactttggtctctgcgtatttctttcttatctagtttccatgctctag

SEQ ID NO: 18 - DNA, 208 bp, synthetic construct
  source 1..208: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 19 - DNA, 145 bp, Adeno associated virus
  source 1..145: mol_type = genomic DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 20 - DNA, 1605 bp, synthetic construct
  source 1..1605: mol_type = other DNA
atggccagcggaggaggcgctcctgtggccgacaacaatgagggagccgacggcgtcggcagctccagcggcaattggcactgcgacagccaatggctgggagatagagtgatcaccaccagcacaagaacctgggctctgcctacatacaacaaccacctgtacaaacagatcagcaactccactagcggtggcagcagcaacgacaatgcctacttcggctacagcaccccttggggatacttcgactttaacagattccactgtcacttctctcctagagattggcagagactgatcaacaacaactggggcttccggcctaagcggcttaacttcaagctgttcaacatccaggtcaaagaggtgacagataacaatggagtgaagaccatcgccaacaacctgacaagcactgtgcaggtgttcaccgacagtgattaccagctgccatacgtgctgggctctgcccacgagggctgcctgcctcctttccctgctgacgtgttcatgatccctcagtatggctacctgaccctgaatgatggctcccaggccgtgggcagaagctccttctactgcctggaatactttcctagccaaatgctgagaacagggaacaacttccagtttagctacgagttcgagaacgtgcccttccacagcagctacgcccattctcaaagcctggacagactgatgaatcccctgatcgaccagtacctgtactacctcagcaagaccatcaacggcagtggccagaaccagcagaccctgaagttcagcgtggcgggcccttctaatatggcagtgcagggccgcaactacatcccaggcccctcctatagacagcagcgggtgtctacaaccgtgacccagaacaacaactcagagtttgcctggcctggcgcctctagctgggccctgaacggcagaaatagcctgatgaaccctggccccgctatggccagccacaaggaaggcgaggaccggttcttccccctgagcggctctctgattttcggcaagcagggcaccggcagggacaacgtggacgccgataaggtgatgatcaccaacgaggaagaaatcaagacaaccaatcctgtggccaccgagagctacggccaggtcgccacaaatcaccagagcgcccaggctcaggcccaaacaggctgggttcagaatcagggcatcctgcccggcatggtgtggcaggacagagatgtgtacctgcaaggccctatctgggccaagatcccccacaccgacggaaacttccaccccagccctctcatgggcggctttggcatgaagcaccctccaccacagatcctgatcaagaacacacctgtgccagccgatcctcctaccgcctttaacaaggacaagctgaacagcttcatcacacagtacagcaccggacaagtgtccgtggaaatcgagtgggagctgcagaaggaaaacagcaaaagatggaacccggaaattcagtacaccagcaactactacaaaagcaacaacgtggaattcgccgtgaacaccgagggagtgtattctgagcctcggcccatcggaaccagatacctgacccggaacctgtga

SEQ ID NO: 21 - protein, 534 aa, Adeno associated virus
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 22 - DNA, 2217 bp, synthetic construct
  source 1..2217: mol_type = other DNA
atggcggctgatggctatctgcctgattggctggaagataatctgagtgagggaatccgggaatggtgggacctgaaacctggcgcccctaagcccaaggccaatcagcagaaacaggacaatggcagaggcctggtgctgccaggctacaaataccttggccctttcaatggcctggataagggcgagccagtgaacgccgctgatgctgccgctctggaacacgacaaggcctacgaccagcaactgcaggccggtgacaacccttacctgagatacaatcacgccgatgccgagttccaggagagactgcaggaggacaccagcttcggaggcaacctgggaagagccgtgttccaggctaagaagagagtgctggagcctctaggactagtggaaagcccagtgaagacagcccctggaaagaaaagacccgtggaaccttcccctcagagaagccccgacagctccaccggcatcggaaaaaagggccagcagcctgccaagaagcgcctcaacttcggacagaccggcgatagcgagtctgtccccgatcctcagcctattggcgaacctcccgccggacctagcggcctgggctctggcaccatggccgccggcggaggcgcccctatggctgacaacaacgagggcgctgacggcgttggcagctcttctggcaactggcactgcgactcgacatggctgggcgaccgggtgatcacaaccagcacaagaacctgggccctgcccacctacaacaaccacctgtacaagcagatcagcaatggcacctctggcggctctacaaacgacaacacctatttcggctattctacaccttggggctacttcgacttcaacagatttcattgtcacttcagccctcgggactggcagcggctgatcaacaacaactggggtttccggcctaagcggctgaactttaagctgttcaacatccaggtcaaagaggtgacacagaatgagggcaccaagaccatcgccaacaatctgaccagcaccatccaagtcttcaccgacagcgagtaccagctgccgtacgtgctgggctccgcccaccagggttgtctgcctccttttccagccgacgtgtttatgatccctcagtatggctacctgaccctgaacaacggctctcaggctgtgggccggagcagcttctactgcctggaatacttccctagccaaatgctgcggaccggcaacaacttcgagttcagctacaatttcgaggacgtgcccttccacagctcctacgcccacagccagagcctggacagactgatgaacccccttatcgaccagtacctctactacctgagcagaacccaaagcaccggcggaaccgccggcacccagcagctgctgttttcccaggccggccccaacaacatgagcgcccaggccaagaactggctgcctggcccctgctaccggcagcaaagagtgtccacaaccctgtcccagaacaacaattctaattttgcctggaccggcgccacaaagtaccacctgaacggcagggacagcctggtgaaccccggcgtggccatggccacccacaaggatgatgaggaaagattcttcccttctagcggcgtcctgatgttcggcaaacagggagccggcaaggacaacgtggactacagcagtgtgatgctgacatctgaggaagaaattaagaccaccaaccccgtggctacagaacagtacggagtggtggccgacaacctgcagcagcaaaacgccgcccctatcgttggagccgtgaacagccagggcgctctgcccggcatggtgtggcagaacagagatgtatacctgcagggccctatctgggccaagatcccccacaccgatggcaacttccatccttcccctctgatgggcggatttggactgaagcacccccctccacaaatcctgatcaagaacacccctgtgccagccgaccctcctaccacattcaaccaagccaaactggccagctttatcacccagtacagcacaggccaggtgtccgtggaaatcgagtgggagctgcagaaggaaaacagcaagagatggaacccagagatccagtacaccagcaactactacaagagcacgaacgtggacttcgccgtgaacacagagggcacatacagcgagccaaggcctatcggcaccagatacctgacaagaaatctgtga

SEQ ID NO: 23 - protein, 738 aa, Adeno associated virus
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 24 - DNA, 158 bp, synthetic construct
  source 1..158: mol_type = other DNA
ttacatgttaatcaataaaccggttaattcgtttcagttgaactttggtctcctgtccttcttatcttatcggttaccatagaaactggttacttattaactgcttggtgcgcttcgcgataaaagacttacgtcatcgggttacccctagtgatgga

SEQ ID NO: 25 - DNA, 2241 bp, synthetic construct
  source 1..2241: mol_type = other DNA
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactatcggagtgtccctcggaggaggacagcagttgctattttctcaggccgggcctaacaacatgtcggctcaggccaagaactggctacccggtccctgctaccggcagcaacgcgtctccacgacactgtcgcagaacaacaacagcaacatcgcctggacgcgggccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaagagaacagaagaggcgacttcaacaaccagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactgctacaaatctacaaatgtggactttgctgtcaatactgagggtacttattccgagcctcgccccattggcacccgttacctcacccgtaatctgtaa

SEQ ID NO: 26 - protein, 746 aa, Adeno associated virus
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTIGVSLGGGQQLLFSQAGPNNMSAQAKNWLPGPCYRQQRVSTTLSQNNNSNIAWTRATKYHLNGRDSLVNPGVAMATHKDDEERFFPSSGVLMFGKQGAGKDNVDYSSVMLTSEEEIKTTNPVATEQYGVVADNLQENRRGDFNNQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNCYKSTNVDFAVNTEGTYSEPRPIGTRYLTRNL

SEQ ID NO: 27 - DNA, 600 bp, synthetic construct
  source 1..600: mol_type = other DNA
caccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggc

SEQ ID NO: 28 - DNA, 660 bp, synthetic construct
  source 1..660: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaatcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 29 - DNA, 2211 bp, Adeno associated virus
  source 1..2211: mol_type = genomic DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 30 - DNA, 208 bp, synthetic construct; identical to SEQ ID NO: 18
  source 1..208: mol_type = other DNA

SEQ ID NO: 31 - DNA, 600 bp, synthetic construct
  source 1..600: mol_type = other DNA
actagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctct

SEQ ID NO: 32 - DNA, 590 bp, synthetic construct
  source 1..590: mol_type = other DNA
ctcatcttgtgcctgctggacttccaccgttgtttcatgtatctcgttagctgagatagcacttcctcctgcccttaccctttatctggctcttagctcctgaaaactgcattattagcttcctcttttgcctctactcttactcaaccaaaattgttttaagatctgtggatctagcttctgctgtgctattcttaggaacacttttatttcctcttagctccatctcaccagttattggctaatggctttgcttggtacctacatctgtacatttctttcgtactagcttctagactgaaaaaggactgttggttcaacatgaaagggaaggaggtaaaagaggacacacaggaaagatggattgggattcaggtctctgctgttgttacttgagattgctttctagattctacttgtggaaacaaaaagcctttgcgagaattctaaactggagtatttctgtaattgaggagtcttgctcagcaaatcccacttaggggactaatgaagtaccaggaagagacagaccatgctcaatccacaaagccaggttttactgaaatgtgacctactttcttatgttcctg

SEQ ID NO: 33 - DNA, 609 bp, synthetic construct
  source 1..609: mol_type = other DNA
aagtttagatcagggtgggcagctctgggttttataggctacactgttaacactcaggctgttttctaccgtttagtcaaaatatagtcaccttgcctgcttcacctgtccatcagagaatggcctcattaattgactctctagtatgaagtcaaagtagctttggtggccctaaatggacaagtatcaagagactgggtgaattgaggagcttgagactgtcacctcagatcgaaaagactgaaaaatcacctcagatcaaaaagactgaaaaatcttcagtctggaaaggggactcaaaaccataattagagtattctggtagaatccttttctccactgttattcatacagttaaggtgaataactaaaagtaattgtgagctgaggagtaagatacaacacacaaggaatcagttaacagagtctcgagtgaaattataaatggaaagaattatgacttgaatcataactctgaggccccattttccctaacaacttttgtcccaataaacgtgggtatttgtttgggagaaactatcatatacatgattacccagtaaacagactgtttactaagtgggtttaattttagaaattgcgcgctgc